(19) United States
(12) Patent Application Publication
(10) Pub. No.: US 2025/0070107 A1
(43) Pub. Date: Feb. 27, 2025

(54) Electrode Comprising a Plurality of Deformable, Proximally-Diverging Electroconductive Protrusions

(71) Applicant: Robert A. Connor, Wyoming, MN (US)

(72) Inventor: Robert A. Connor, Wyoming, MN (US)

(73) Assignee: Medibotics LLC, Ham Lake, MN (US)

(21) Appl. No.: 18/902,821

(22) Filed: Sep. 30, 2024

Related U.S. Application Data
(63) Continuation-in-part of application No. 18/748,059, filed on Jun. 19, 2024.
(63) Continuation-in-part of application No. 18/411,540, filed on Jan. 12, 2024.
(63) Continuation-in-part of application No. 18/219,684, filed on Jul. 9, 2023.
Application No. 18/748,059 is a continuation-in-part of application No. 18/411,540, filed on Jan. 12, 2024.
Application No. 18/411,540 is a continuation-in-part of application No. 18/219,684, filed on Jul. 9, 2023.
Application No. 18/411,540 is a continuation-in-part of application No. 18/219,684, filed on Jul. 9, 2023.
Application No. 18/219,684 is a continuation-in-part of application No. 17/714,988, filed on Apr. 6, 2022, now Pat. No. 11,850,052.
Application No. 17/714,988 is a continuation-in-part of application No. 16/838,541, filed on Apr. 2, 2020, now abandoned.
Application No. 17/714,988 is a continuation-in-part of application No. 17/665,086, filed on Feb. 4, 2022, now Pat. No. 11,662,819.
Application No. 17/665,086 is a continuation-in-part of application No. 17/136,117, filed on Dec. 29, 2020, now abandoned.
Application No. 17/136,117 is a continuation-in-part of application No. 16/554,029, filed on Aug. 28, 2019, now abandoned.
Application No. 17/665,086 is a continuation-in-part of application No. 17/136,117, filed on Dec. 29, 2020, now abandoned.
Application No. 17/136,117 is a continuation-in-part of application No. 16/554,029, filed on Aug. 28, 2019, now abandoned.
Application No. 17/136,117 is a continuation-in-part of application No. 16/838,541, filed on Apr. 2, 2020, now abandoned.
Application No. 16/838,541 is a continuation-in-part of application No. 16/737,052, filed on Jan. 8, 2020, now Pat. No. 11,754,542.
Application No. 16/737,052 is a continuation-in-part of application No. 16/568,580, filed on Sep. 12, 2019, now Pat. No. 11,478,158.
Application No. 16/568,580 is a continuation-in-part of application No. 16/554,029, filed on Aug. 28, 2019, now abandoned.
Application No. 16/554,029 is a continuation-in-part of application No. 16/554,029, filed on Aug. 28, 2019, now abandoned.
Application No. 16/554,029 is a continuation-in-part of application No. 15/236,401, filed on Aug. 13, 2016, now abandoned.
Application No. 16/737,052 is a continuation-in-part of application No. 16/568,580, filed on Sep. 12, 2019, now Pat. No. 11,478,158.
Application No. 16/568,580 is a continuation-in-part of application No. 15/963,061, filed on Apr. 25, 2018, now Pat. No. 10,772,559.
Application No. 16/568,580 is a continuation-in-part of application No. 15/963,061, filed on Apr. 25, 2018, now Pat. No. 10,772,559.
Application No. 15/963,061 is a continuation-in-part of application No. 16/022,987, filed on Jun. 29, 2018, now Pat. No. 11,172,859.
Application No. 16/022,987 is a continuation-in-part of application No. 15/136,948, filed on Apr. 24, 2016, now Pat. No. 10,234,942.
Application No. 15/963,061 is a continuation-in-part of application No. 15/464,349, filed on Mar. 21, 2017, now Pat. No. 9,968,297.
Application No. 15/464,349 is a continuation-in-part of application No. 15/136,948, filed on Apr. 24, 2016, now Pat. No. 10,234,942.
Application No. 15/136,948 is a continuation-in-part of application No. 14/562,719, filed on Dec. 7, 2014, now Pat. No. 10,130,277.
Application No. 14/562,719 is a continuation-in-part of application No. 14/330,649, filed on Jul. 14, 2014, now abandoned.
Application No. 15/236,401 is a continuation-in-part of application No. 15/136,948, filed on Apr. 24, 2016, now Pat. No. 10,234,942.
Application No. 15/136,948 is a continuation-in-part of application No. 14/599,522, filed on Jan. 18, 2015, now Pat. No. 9,814,426.
Application No. 14/599,522 is a continuation-in-part of application No. 14/599,522, filed on Jan. 18, 2015, now Pat. No. 9,814,426.
Application No. 14/599,522 is a continuation-in-part of application No. 14/562,719, filed on Dec. 7, 2014, now Pat. No. 10,130,277.
Application No. 14/330,649 is a continuation-in-part of application No. 13/797,955, filed on Mar. 12, 2013, now Pat. No. 9,456,916.
Application No. 13/797,955 is a continuation-in-part of application No. 13/523,739, filed on Jun. 14, 2012, now Pat. No. 9,042,596.
(60) Provisional application No. 62/972,692, filed on Feb. 11, 2020.
(60) Provisional application No. 62/972,692, filed on Feb. 11, 2020.
(60) Provisional application No. 62/851,904, filed on May 23, 2019.
(60) Provisional application No. 62/796,901, filed on Jan. 25, 2019.
(60) Provisional application No. 62/791,838, filed on Jan. 13, 2019.
(60) Provisional application No. 62/430,667, filed on Dec. 6, 2016.
(60) Provisional application No. 62/322,594, filed on Apr. 14, 2016.
(60) Provisional application No. 62/303,126, filed on Mar. 3, 2016.
(60) Provisional application No. 62/169,661, filed on Jun. 2, 2015.
(60) Provisional application No. 62/160,172, filed on May 12, 2015.
(60) Provisional application No. 62/089,696, filed on Dec. 9, 2014.
(60) Provisional application No. 62/017,615, filed on Jun. 26, 2014.
(60) Provisional application No. 61/939,244, filed on Feb. 12, 2014.
(60) Provisional application No. 61/932,517, filed on Jan. 28, 2014.
(60) Provisional application No. 61/932,517, filed on Jan. 28, 2014.
(60) Provisional application No. 61/729,494, filed on Nov. 23, 2012.

(51) Int. Cl. H01M 4/02; A61B 5/256

(52) U.S. Cl. CPC H01M 4/02; A61B 5/256; A61B 2562/046; H01M 2004/021; H01M 2004/024; H01M 2004/025

(57) ABSTRACT

An electrode can comprise a plurality of flexible longitudinal electroconductive protrusions which deform when pressed against a person's head. Proximal ends of the protrusions are adjacent to each other and have a first degree of curvature in a pre-deformation configuration. Proximal ends of the protrusions are apart from each other and have a second degree of curvature in a post-deformation configuration. The proximal-divergence of the ends of the protrusions enables them to slide between strands of hair to enable good electrical communication with the surface of the person's head.

### CROSS-REFERENCE TO RELATED APPLICATIONS

This patent application is a continuation-in-part of U.S. patent application Ser. No. 18/748,059 filed on 2024 Jun. 19. This patent application is a continuation-in-part of U.S. patent application Ser. No. 18/411,540 filed on 2024 Jan. 12. This patent application is a continuation-in-part of U.S. patent application Ser. No. 18/219,684 filed on 2023 Jul. 9.

U.S. patent application Ser. No. 18/748,059 was a continuation-in-part of U.S. patent application Ser. No. 18/411,540 filed on 2024 Jan. 12. U.S. patent application Ser. No. 18/748,059 was a continuation-in-part of U.S. patent application Ser. No. 18/219,684 filed on 2023 Jul. 9. U.S. patent application Ser. No. 18/411,540 was a continuation-in-part of U.S. patent application Ser. No. 18/219,684 filed on 2023 Jul. 9. U.S. patent application Ser. No. 18/219,684 was a continuation-in-part of U.S. patent application Ser. No. 17/714,988 filed on 2022 Apr. 6. U.S. patent application Ser. No. 18/219,684 was a continuation-in-part of U.S. patent application Ser. No. 16/838,541 filed on 2020 Apr. 2.

U.S. patent application Ser. No. 17/714,988 was a continuation-in-part of U.S. patent application Ser. No. 17/665,086 filed on 2022 Feb. 4. U.S. patent application Ser. No. 17/714,988 was a continuation-in-part of U.S. patent application Ser. No. 17/136,117 filed on 2020 Dec. 29. U.S. patent application Ser. No. 17/714,988 was a continuation-in-part of U.S. patent application Ser. No. 16/554,029 filed on 2019 Aug. 28. U.S. patent application Ser. No. 17/665,086 was a continuation-in-part of U.S. patent application Ser. No. 17/136,117 filed on 2020 Dec. 29. U.S. patent application Ser. No. 17/665,086 was a continuation-in-part of U.S. patent application Ser. No. 16/554,029 filed on 2019 Aug. 28.

U.S. patent application Ser. No. 17/136,117 was a continuation-in-part of U.S. patent application Ser. No. 16/838,541 filed on 2020 Apr. 2. U.S. patent application Ser. No. 17/136,117 claimed the priority benefit of U.S. provisional patent application 62/972,692 filed on 2020 Feb. 11. U.S. patent application Ser. No. 17/136,117 was a continuation-in-part of U.S. patent application Ser. No. 16/737,052 filed on 2020 Jan. 8. U.S. patent application Ser. No. 17/136,117 was a continuation-in-part of U.S. patent application Ser. No. 16/568,580 filed on 2019 Sep. 12. U.S. patent application Ser. No. 17/136,117 was a continuation-in-part of U.S. patent application Ser. No. 16/554,029 filed on 2019 Aug. 28.

U.S. patent application Ser. No. 16/838,541 claimed the priority benefit of U.S. provisional patent application 62/972,692 filed on 2020 Feb. 11. U.S. patent application Ser. No. 16/838,541 was a continuation-in-part of U.S. patent application Ser. No. 16/554,029 filed on 2019 Aug. 28. U.S. patent application Ser. No. 16/838,541 claimed the priority benefit of U.S. provisional patent application 62/851,917 filed on 2019 May 23. U.S. patent application Ser. No. 16/838,541 claimed the priority benefit of U.S. provisional patent application 62/837,712 filed on 2019 Apr. 23. U.S. patent application Ser. No. 16/838,541 was a continuation-in-part of U.S. patent application Ser. No. 15/236,401 filed on 2016 Aug. 13. U.S. patent application Ser. No. 16/737,052 was a continuation-in-part of U.S. patent application Ser. No. 16/568,580 filed on 2019 Sep. 12. U.S. patent application Ser. No. 16/737,052 was a continuation-in-part of U.S. patent application Ser. No. 15/963,061 filed on 2018 Apr. 25. U.S. patent application Ser. No. 16/568,580 was a continuation-in-part of U.S. patent application Ser. No. 15/963,061 filed on 2018 Apr. 25.

U.S. patent application Ser. No. 16/554,029 claimed the priority benefit of U.S. provisional patent application 62/851,904 filed on 2019 May 23. U.S. patent application Ser. No. 16/554,029 claimed the priority benefit of U.S. provisional patent application 62/796,901 filed on 2019 Jan. 25. U.S. patent application Ser. No. 16/554,029 claimed the priority benefit of U.S. provisional patent application 62/791,838 filed on 2019 Jan. 13. U.S. patent application Ser. No. 16/554,029 was a continuation-in-part of U.S. patent application Ser. No. 16/022,987 filed on 2018 Jun. 29. U.S. patent application Ser. No. 16/022,987 was a continuation-in-part of U.S. patent application Ser. No. 15/136,948 filed on 2016 Apr. 24. U.S. patent application Ser. No. 15/963,061 was a continuation-in-part of U.S. patent application Ser. No. 15/464,349 filed on 2017 Mar. 21.

U.S. patent application Ser. No. 15/464,349 claimed the priority benefit of U.S. provisional patent application 62/430,667 filed on 2016 Dec. 6. U.S. patent application Ser. No. 15/464,349 was a continuation-in-part of U.S. patent application Ser. No. 15/136,948 filed on 2016 Apr. 24. U.S. patent application Ser. No. 15/464,349 was a continuation-in-part of U.S. patent application Ser. No. 14/562,719 filed on 2014 Dec. 7. U.S. patent application Ser. No. 15/464,349 was a continuation-in-part of U.S. patent application Ser. No. 14/330,649 filed on 2014 Jul. 14. U.S. patent application Ser. No. 15/236,401 was a continuation-in-part of U.S. patent application Ser. No. 15/136,948 filed on 2016 Apr. 24. U.S. patent application Ser. No. 15/236,401 was a continuation-in-part of U.S. patent application Ser. No. 14/599,522 filed on 2015 Jan. 18.

U.S. patent application Ser. No. 15/136,948 claimed the priority benefit of U.S. provisional patent application 62/322,594 filed on 2016 Apr. 14. U.S. patent application Ser. No. 15/136,948 claimed the priority benefit of U.S. provisional patent application 62/303,126 filed on 2016 Mar. 3. U.S. patent application Ser. No. 15/136,948 claimed the priority benefit of U.S. provisional patent application 62/169,661 filed on 2015 Jun. 2. U.S. patent application Ser. No. 15/136,948 claimed the priority benefit of U.S. provisional patent application 62/160,172 filed on 2015 May 12. U.S. patent application Ser. No. 15/136,948 was a continuation-in-part of U.S. patent application Ser. No. 14/599,522 filed on 2015 Jan. 18.

U.S. patent application Ser. No. 14/599,522 claimed the priority benefit of U.S. provisional patent application 62/089,696 filed on 2014 Dec. 9. U.S. patent application Ser. No. 14/599,522 was a continuation-in-part of U.S. patent application Ser. No. 14/562,719 filed on 2014 Dec. 7. U.S. patent application Ser. No. 14/599,522 claimed the priority benefit of U.S. provisional patent application 62/017,615 filed on 2014 Jun. 26. U.S. patent application Ser. No. 14/599,522 claimed the priority benefit of U.S. provisional patent application 61/939,244 filed on 2014 Feb. 12. U.S. patent application Ser. No. 14/599,522 claimed the priority benefit of U.S. provisional patent application 61/932,517 filed on 2014 Jan. 28.

U.S. patent application Ser. No. 14/562,719 claimed the priority benefit of U.S. provisional patent application 61/932,517 filed on 2014 Jan. 28. U.S. patent application Ser. No. 14/330,649 was a continuation-in-part of U.S. patent application Ser. No. 13/797,955 filed on 2013 Mar. 12. U.S. patent application Ser. No. 14/330,649 was a continuation-in-part of U.S. patent application Ser. No. 13/523,739 filed on 2012 Jun. 14. U.S. patent application Ser. No. 13/797,955 claimed the priority benefit of U.S. provisional patent application 61/729,494 filed on 2012 Nov. 23.

The entire contents of these applications are incorporated herein by reference.

### FEDERALLY SPONSORED RESEARCH

Not Applicable

### SEQUENCE LISTING OR PROGRAM

Not Applicable

### BACKGROUND 13 FIELD OF INVENTION

This invention relates to dry electrodes.

### INTRODUCTION

There are numerous potential applications for incorporating electrodes into technology worn on a person's head—such as smart eyewear, smart earwear, smart headbands, and smart adhesive patches. However, wet electrodes are not well-suited for use with such wearable technology and flat dry electrodes do not work well on areas of a person's head which are covered by hair. There has been some creative innovation in the prior art in the development of dry electrodes with protrusions which penetrate between strands of hair. However, protrusions in the prior art which are sufficiently resilient and narrow to penetrate hair can be uncomfortable, cause skin irritation, and lack sufficient contact area to enable good electromagnetic communication with a person's head. There remains an unmet need for dry electrodes which can be incorporated into wearable technology and work well on areas of a person's head which are covered by hair.

### REVIEW OF THE RELEVANT ART

In the patent literature, U.S. patent application No. 20090134887 (Hu et al., May 28, 2009, “Contact Sensor”) discloses a sensor with a main body and arc-shaped conductors. U.S. patent application 20130274583 (Heck, Oct. 17, 2013, “Electrodes Adapted for Transmitting or Measuring Voltages Through Hair”) discloses a composite metal electrode with raised points which pass through hair to contact skin. U.S. patent applications 20140024912 (Dalke, Jan. 23, 2014, “Neurophysiological Dry Sensor”) and 20150265176 (Dalke, Sep. 24, 2015, “Neurophysiological Dry Sensor”) disclose a sensor with conductive spires. U.S. patent application No. 20140107458 (Op De Beeck et al., Apr. 17, 2014, “Resilient Sensor for Biopotential Measurements”) discloses a sensor with a cavity wherein an electrical contacting unit is partially secured.

U.S. Pat. No. 8,798,710 (Chi, Aug. 5, 2014, “Apparatuses, Systems and Methods for Biopotential Sensing with Dry Electrodes”) discloses an electrode with an electrical conductor, a membrane permeable to ionic conduction, and a conductive medium in communication with a portion of the electrical conductor and a portion of the membrane. U.S. patent application No. 20140288406 (Chai, Sep. 25, 2014, “Line-Contact Dry Electrode”) discloses a line-contact dry electrode with elastic conductive branches forming a comb-like electrode. U.S. patent application No. 20150088224 (Goldwasser et al., Mar. 26, 2015, “Wearable Transdermal Electrical Stimulation Devices and Methods of Using Them”) discloses an electrode with first and second transdermal electrodes. U.S. patent application No. 20150141788 (Chi et al., May 21, 2015, “Transducer Assemblies for Dry Applications of Transducers”) and U.S. Pat. No. 9,314,183 (Chi et al., Apr. 19, 2016, “Transducer Assemblies for Dry Applications of Transducers”) disclose at least one probe extending from a terminal to penetrate through patches of hair.

U.S. patent application No. 20150367122 (Morshed et al., Dec. 24, 2015, “Patterned Carbon Nanotube Electrode”) discloses an electrode with carbon nanotube pillars. U.S. patent application 20160089045 (Sadeghian-Motahar et al., Mar. 31, 2016, “Bio-Potential Sensing Materials as Dry Electrodes and Devices”) discloses a dry electrode in contact with skin to receive bio-potential signals. U.S. patent application No. 20160143554 (Lim et al., May 26, 2016, “Apparatus for Measuring Bioelectrical Signals”) discloses a sensor electrode with a tapering portion and a protruding portion that extends from the tapering portion. U.S. patent application No. 20160174859 (Oudenhoven et al., Jun. 23, 2016, “Electrode for Biopotential Sensing”) discloses an electrode base and a plurality of mesh pins protruding from the electrode base.

U.S. patent application No. 20170112444 (Lin et al., Apr. 27, 2017, “Bio-Signal Sensor”) discloses a dry electrode with probes and contacts correspondingly and electrically connected to the probes, wherein each of the probes senses and transmits an electrical signal to the corresponding contact. U.S. patent application No. 20170150925 (Jung, Jun. 1, 2017, “EEG Hair Band”) discloses EEG sensors embodied in a hair band. U.S. patent application No. 20170172447 (Mitra et al., Jun. 22, 2017, “Sensor, System, and Holder Arrangement for Biosignal Activity Measurement”) discloses a sensor with a pins protruding from an electrode base. U.S. patent application No. 20170224990 (Goldwasser et al., Aug. 10, 2017, “Apparatuses and Methods for Neuromodulation”) discloses apparatuses and methods to apply an ensemble current waveform between two or more electrodes.

U.S. patent application No. 20170258353 (Jovanovic et al., Sep. 14, 2017, “Headsets and Electrodes for Gathering Electroencephalographic Data”) discloses electrodes with a housing, a spring, and a pin. U.S. patent application No. 20170258400 (Jovanovic et al., Sep. 14, 2017, “Headsets and Electrodes for Gathering Electroencephalographic Data”) discloses an electrode with a ring disposed in an opening and an arm, where the arm has a first portion extending outward from the opening away from the housing and a second portion extending from an end of the first portion toward the housing and into the cavity, and the first and second portions connect at a bend.

U.S. Pat. No. 9,820,670 (Parvizi et al., Nov. 21, 2017, “Methods and Apparatus for Electrode Placement and Tracking”) and 10888240 (Parvizi et al., Jan. 12, 2021, “Methods and Apparatus for Electrode Placement and Tracking”) as well as U.S. patent applications 20220117536 (Parvizi et al., Apr. 21, 2022. “Methods and Apparatus for Electrode Placement and Tracking”) and 20220117535 (Parvizi et al., Apr. 21, 2022, “Methods and Apparatus for Electrode Placement and Tracking”) disclose tubular members extending from an electrode body. U.S. patent application No. 20180049639 (Tian, Feb. 22, 2018, “Dry Electrode, Its Manufacturing Method and Bio-Electromagnetic Wave Detecting Device and Sensor Element Comprising the Dry Electrode”) discloses a dry electrode with a protruding structure comprising an inner core made of a flexible insulating material and a conductive thin film coated on an outer side of the inner core.

U.S. patent applications 20180153470 (Gunasekar et al., Jun. 7, 2018, “Electroencephalography Headset and System for Collecting Biosignal Data”), 20200281527 (Gunasekar et al., Sep. 10, 2020, “Electroencephalography Headset and System for Collecting Biosignal Data”), 20220015701 (Gunasekar et al., Jan. 20, 2022, “Electroencephalography Headset and System for Collecting Biosignal Data”), and 20220022813 (Gunasekar et al., Jan. 27, 2022, “Electroencephalography Headset and System for Collecting Biosignal Data”) disclose a system for collecting biosignal data including: a first electrode fixedly mounted to a first band and centered between left and right junctions; a second electrode mounted to the first band offset from the first electrode and laterally-adjustable along the length of the first band; and a third electrode mounted to a second band and laterally-adjustable along the length of the second band.

U.S. patent application No. 20180192906 (Soulet De Brugiere et al., Jul. 12, 2018, “Polymer Composition and Electrode for a Device for the Non-Invasive Measurement of Biological Electrical Signals”) discloses a polymer matrix in which are dispersed carbon nanotubes and adsorbent elements selected from activated carbon particles and graphene nanoplatelets. U.S. patent application No. 20180235499 (Zorman et al., Aug. 23, 2018, “Method for Measuring an Electrophysiological Parameter by Means of a Capacitive Electrode Sensor of Controlled Capacitance”) discloses a sensor with a base and a plurality of protrusions projecting from the base. U.S. patent application No. 20180235500 (Lee et al., Aug. 23, 2018, “Dry Electrode for Detecting Biosignal and Method for Manufacturing Same”) discloses a dry electrode comprising a protrusion and a coating on an end surface of the protrusion.

U.S. patent applications 20180348863 (Aimone et al., Dec. 6, 2018, “Wearable Computing Device With Electrophysiological Sensors”), 20200019243 (Aimone et al., Jan. 16, 2020, “Wearable Computing Device With Electrophysiological Sensors”), and 20210200313 (Aimone et al., Jul. 1, 2021, “Wearable Computing Device With Electrophysiological Sensors”) disclose a wearable computing device with bio-signal sensors and a feedback module providing an interactive mediated reality (“VR”) environment. U.S. patent application No. 20180353096 (Mercier et al., Dec. 13, 2018, “Electrode, Wearable Assembly and System”) and U.S. Pat. No. 10,842,404 (Mercier et al., Nov. 24, 2020, “Electrode, Wearable Assembly and System”) disclose an electrode with a base and a plurality of legs extending from the base. U.S. patent application No. 20190000338 (Van Den Ende et al., Jan. 3, 2019, “Method and System for Obtaining Signals from Dry EEG Electrodes”) discloses an actuator coupled to an EEG electrode and configured to move the electrode in at least two dimensions.

U.S. Pat. No. 10,285,646 (Grant et al., May 14, 2019, “Connection Quality Assessment for EEG Electrode Arrays”) and 10980480 (Grant et al., Apr. 20, 2021, “Connection Quality Assessment for EEG Electrode Arrays”) disclose systems, devices, and methods to assess connection quality between the electrodes of a bioelectrical signal measurement and/or electrical stimulation device and the tissue, typically skin, of a subject. U.S. patent application No. 20190200925 (Aimone et al., Jul. 4, 2019, “Wearable Computing Device”) discloses a wearable device including a flexible band having at least a front portion to contact at least part of a frontal region of a user's head, a rear portion to contact at least part of an occipital region of the user's head, and at least one side portion extending between the front portion and the rear portion to contact at least part of an auricular region of the user's head. U.S. patent application No. 20190239807 (Watson et al., Aug. 8, 2019, “Hair Ratcheting Electroencephalogram Sensors”) discloses a sensor with a locking mechanism which permits one-way axial motion of a thread through a channel from the first side to the second side. U.S. patent application No. 20190328261 (Shakour et al., Oct. 31, 2019, “Brush Electrode”) discloses a plurality of strand electrodes extending outward from an electrode base.

U.S. patent applications 20190365270 (Bachelder et al., Dec. 5, 2019, “Adjustable Geometry Wearable Electrodes”) and 20230000416 (Bachelder et al., Jan. 5, 2023, “Adjustable Geometry Wearable Electrodes”) as well as U.S. Pat. No. 10,433,756 (Bachelder et al., Oct. 8, 2019, “Adjustable Geometry Wearable Electrodes”) and U.S. Pat. No. 11,357,434 (Bachelder et al., Jun. 14, 2022, “Adjustable Geometry Wearable Electrodes”) disclose electrode assemblies which contact a patient's scalp and hair via collapse, compression, or telescoping. U.S. patent application No. 20200069206 (Zaliasl et al., Mar. 5, 2020, “System and a Method for Acquiring an Electrical Signal and a Wearable Device”) discloses a system for acquiring an electrical signal comprising: a plurality of electrodes, a plurality of signal quality detectors, each detector, being configured to detect a signal from a pair of electrodes and each detector comprising an analog-to-digital converter. U.S. patent applications 20200159324 (Keller et al., May 21, 2020, “Headware for Computer Control”) and 20210109594 (Keller et al., Apr. 15, 2021, “Headware for Computer Control”) disclose headware configured to sit at the top of a head and apply pressure to at least one side of the head.

U.S. patent application No. 20200237249 (Gunasekar et al., Jul. 30, 2020, “Headset and Electrodes for Sensing Bioelectrical Potential and Methods of Operation Thereof”) discloses an electrode tip body, one or more deflectable electrode legs coupled to the electrode tip body, and a conductive cushioning material coupled to a segment of at least one of one or more electrode legs. U.S. Pat. No. 10,743,809 (Kamousi et al., Aug. 18, 2020, “Systems and Methods for Seizure Prediction and Detection”) discloses using a machine learning algorithm to perform a seizure binary classification. U.S. patent applications 20200305786 (Grant et al., Oct. 1, 2020, “Systems and Methods for Processing Sonified Brain Signals”) and 20210267539 (Grant et al., Sep. 2, 2021, “Systems and Methods for Processing Sonified Brain Signals”) as well as U.S. Pat. No. 10,849,553 (Grant et al., Dec. 1, 2020, “Systems and Methods for Processing Sonified Brain Signals”) disclose systems and methods for sonifying electrical signals, particularly EEG signals.

U.S. patent application No. 20210038106 (Ramakrishnan et al., Feb. 11, 2021, “Mobile, Wearable EEG Device With High Quality Sensors”) discloses sensor units with conductive segments in a flexible sensing layer. U.S. patent application No. 20210282695 (Goldstein et al., Sep. 16, 2021, “Personal Apparatus for Conducting Electroencephalography”) discloses an apparatus for conducting electroencephalography allowing for secure and easy application to a human subject's forehead. U.S. patent application No. 20210338128 (Le Lous et al., Nov. 4, 2021, “Sensor for Measuring a Biological Potential”) discloses an electrode with a base and at least one leg. U.S. patent application 20210353200 (Xu et al., Nov. 18, 2021, “Electrode for Potential Acquisition of a Surface and Manufacturing Method Thereof”) discloses an electrode with at least two pins.

U.S. patent application No. 20220000407 (Ludwig et al., Jan. 6, 2022, “Dry Electrodes”) discloses an electrode with a substrate, electrically conductive particles in contact with the substrate, a supporting layer that envelopes the electrically conductive particles with points that protrude from the supporting layer, and an electrical connector. U.S. patent application No. 20220004257 (Keller et al., Jan. 6, 2022, “Headware for Computer Control”) discloses headware with a first arm pivotally coupled to a body portion and a second arm pivotably coupled to the body portion. U.S. patent application No. 20220211313 (Lee, Jul. 7, 2022. “Dry Electroencephalographic Electrode”) discloses a dry EEG electrode with a plurality of metal probes. U.S. Pat. No. 11,471,088 (Parvizi et al., Oct. 18, 2022, “Handheld or Wearable Device for Recording or Sonifying Brain Signals”) discloses a handheld device for sonifying electrical signals obtained from a subject.

U.S. patent application No. 20230031613 (Fleury, Feb. 2, 2023, “Wearable Device”) discloses a wearable device with a flexible retention mount to allow rotation of a flexible and extendable body relative to an electronics module and to transfer tension force from the flexible and extendable body to the electronics module. U.S. patent application No. 20230043938 (Kele, Feb. 9, 2023, “Flexible Electroencephalography Headset”) discloses an electrode tip with a thin conductive probe mounted on an elastic beam to extend from a base of the electrode tip, bypass hair, and electrically couple to the head of the user. U.S. patent application No. 20230165503 (Coyle, Jun. 1, 2023, “Flexible Electrical Measurement Apparatus”) discloses an electrode with a central portion and a plurality of legs extending radially outwards from the central portion.

There is also relevant art in the non-patent literature. (Acar, 2019), “Wearable and Flexible Textile Electrodes for Biopotential Signal Monitoring: A Review,” Electronics, 2019, 8(5), 479, presents a systematic review of wearable textile electrodes for physiological signal monitoring. (Casson, 2019), “Wearable EEG and Beyond,” Biomedical Engineering Letters, January 2019, 9(1), 53-71, reviews recent progress on electrodes used to make connections to the head and the physical EEG hardware. (Chen, 2014), “Soft, Comfortable Polymer Dry Electrodes for High Quality ECG and EEG Recording.” Sensors, Dec. 10, 2014, 14(12), 23758-80, discloses dry electrodes fabricated from EPDM rubber containing various additives for optimum conductivity, flexibility and ease of fabrication.

(Chen, 2016), “Polymer-Based Dry Electrodes for Biopotential Measurements,” Thesis, Arenberg Doctoral School, 2016, investigates the mechanical properties of the polymer dry electrodes with compression tests for elastic modulus and compliance characterization. (Chi, 2010), “Dry-Contact and Noncontact Biopotential Electrodes: Methodological Review,” IEEE Reviews in Biomedical Engineering. 2010, 3, 106-119, explores the use of dry/noncontact electrodes for clinical use by explaining the electrical models for dry, insulated and noncontact electrodes and showing performance limits, along with measured data. (Chlaihawi, 2018), “Development of Printed and Flexible Dry ECG Electrodes.” Sensing and Bio-Sensing Research, 2018, 20, 9-15, discloses printed, flexible and wearable dry electrodes for monitoring electrocardiogram (ECG) signals without any skin preparation or wet gel.

(Flumeri, 2019), “The Dry Revolution: Evaluation of Three Different EEG Dry Electrode Types in Terms of Signal Spectral Features, Mental States Classification and Usability.” Sensors, Mar. 19, 2019, 19(6), 1365, compares three different dry electrode types: gold-coated single pin, multiple pins and solid-gel. (Fu. 2020), “Dry Electrodes for Human Bioelectrical Signal Monitoring.” Sensors, Jun. 29, 2020, 20(13), 3651, gives a retrospective overview of the development of dry electrodes used for monitoring bioelectrical signals, including sensing principles, material selection, device preparation, and measurement performance. (Gao, 2018), “Soft Pin-Shaped Dry Electrode with Bristles for EEG Signal Measurements,” Sensors and Actuators, 2018, Vol. 283, 348-361, presents a novel soft pin-shaped dry electrode for electroencephalography recording.

(Hsu, 2014), “Developing Barbed Microtip-Based Electrode Arrays for Biopotential Measurement,” Sensors, 2014, 14(7), 12370-12386, discloses the fabrication of barbed microtip-based electrode arrays via silicon wet etching. (Kocturova, 2019), “Comparison of Dry Electrodes for Mobile EEG System,” 2019, evaluates two types of comb electrodes: one based on a Ag—AgCl alloy and one based on a flexible conductive polymer. (Krachunov, 2016), “3D Printed Dry EEG Electrodes,” Sensors, 2016, 16(10), 1635, presents a novel methodology for the design and manufacture of dry electrodes using low cost desktop 3D printers.

(Lau-Zhu, 2019), “Mobile EEG in Research on Neurodevelopmental Disorders: Opportunities and Challenges.” Developmental Cognitive Neuroscience, 2019, Vol. 36, presents a brief overview of recent developments in mobile EEG technologies. (Lee, 2015), “Reverse-Curve-Arch-Shaped Dry EEG Electrode for Increased Skin-Electrode Contact Area on Hairy Scalps,” Electronics Letters, Oct. 1, 2015, discloses reverse-curve-arch-shaped dry EEG electrodes for use in increasing the skin-electrode contact area on hairy scalps. (Lopez-Gordo, 2014), “Dry EEG Electrodes,” Sensors, Jul. 18, 2014, 14(7), 12847-70, reviews current approaches to developing dry EEG electrodes for clinical and other applications.

(Mota, 2013), “Development of a Quasi-Dry Electrode for EEG Recording.” Sensors and Actuators, 2013, Vol. 199, 310-317, reports on the development of a novel polymer-based electrode prototype for electroencephalography (EEG) between classic “wet” and “dry” electrodes. (Olesen, 2020), “Development and Assessment of Electrodes and Instrumentation for Plantar Skin Impedance Measurements,” Thesis, Master in Electronics, Informatics and Technology, University of Oslo, Autumn, 2020, describes the development and testing of electrodes for plantar bioimpedance measurements. (Ouyang, 2021), “Application of Intrinsically Conducting Polymers in Flexible Electronics,” SmartMat, Aug. 18, 2021, 2, discusses the use of intrinsically conducting polymers (ICPs), such as polyacetylene, polyaniline, polypyrrole, polythiophene, and poly(3,4-ethylenedioxythiophene) (PEDOT) for dry electrodes.

(Ruffini, 2008), “First Human Trials of a Dry Electrophysiology Sensor Using a Carbon Nanotube Array Interface,” Sensors and Actuators, Jun. 15, 2008, 144, reports the results from the first human trials of a new dry electrode sensor for surface biopotential applications, wherein the contact surface of the electrode is covered with carbon nanotubes. (Shad, 2020), “Impedance and Noise of Passive and Active Dry EEG Electrodes: A Review,” IEEE Sensors Journal, Jul. 27, 2020, reviews the impedance and noise of passive and active dry EEG electrodes. (Sunwoo, 2020), “Advances in Soft Bioelectronics for Brain Research and Clinical Neuroengineering,” Matter, 2020, 3(6) 1923-1947, reviews recent technological advances using unconventional soft materials, such as silicon/metal nanowires, functionalized hydrogels, and stretchable conductive nanocomposites. (Zhang, 2020), “Fully Organic Compliant Dry Electrodes Self-Adhesive to Skin for Long-Term Motion-Robust Epidermal Biopotential Monitoring,” Nature Communications, 2020, 11, 4683, reports an intrinsically conductive polymer dry electrode with excellent self-adhesiveness, stretchability, and conductivity.

Despite the above creative innovation and progress in this field, there still remains a need for better dry EEG electrode designs which can more-effectively penetrate between strands of hair to ensure good electrical communication with a person's head.

### SUMMARY OF THE INVENTION

This invention is an electrode for use on a person's head which comprises a plurality of flexible longitudinal electroconductive protrusions which deform when pressed against the surface of the person's head. The proximal ends of the protrusions are adjacent to each other and the protrusions have a first degree of curvature in the pre-deformation configuration. The proximal ends of the protrusions are apart from each other and the protrusions have a second degree of curvature (which is greater than the first degree of curvature) in the post-deformation configuration. The proximal-divergence of the ends of the protrusions enables them to slide between strands of hair to enable good electrical communication with the surface of the person's head. Accordingly, this electrode design is especially advantageous for use on a hair-covered portion of a person's head.

### BRIEF INTRODUCTION TO THE FIGURES

FIG. 1 shows an electrode with a convex base and electroconductive protrusions.

FIG. 2 shows an electrode with a polygonal base and electroconductive protrusions.

FIG. 3 shows an electrode with a base and electroconductive protrusions, wherein the base is curved in a plane perpendicular to the surface of a person's head.

FIG. 4 shows an electrode with a flexible base and electroconductive protrusions, wherein the curvature of the base changes.

FIG. 5 shows an electrode with a flexible base and electroconductive protrusions, wherein tips of protrusions spread apart when the curvature of the base changes.

FIG. 6 shows an electrode with a base with moveable sections and electroconductive protrusions, wherein the moveable sections become more co-planar.

FIG. 7 shows an electrode with a base with moveable sections and electroconductive protrusions, wherein tips of protrusions spread apart when the moveable sections become more co-planar.

FIG. 8 shows an electrode with a base and electroconductive protrusions with arcuate cross sections.

FIG. 9 shows an electrode with a base and electroconductive protrusions with polygonal cross sections.

FIG. 10 shows an electrode with a base and tapered electroconductive protrusions.

FIG. 11 shows an electrode with a base and conic, frustal, or funnel shaped electroconductive protrusions

FIG. 12 shows an electrode with a base and conic-section-shaped electroconductive protrusions.

FIG. 13 shows an electrode with a base and paraboloidal-shaped electroconductive protrusions.

FIG. 14 shows an electrode with a base and electroconductive protrusions, wherein the cross section of a proximal section of a protrusion is larger than that of a distal section.

FIG. 15 shows an electrode with a base and electroconductive protrusions, wherein the cross section of a proximal section of a protrusion is smaller than that of a distal section.

FIG. 16 shows an electrode with a base and electroconductive protrusions, wherein there is a 90-degree angle between proximal and distal sections of a protrusion.

FIG. 17 shows an electrode with a base and electroconductive protrusions, wherein there is an obtuse angle between a proximal section of the protrusion and a distal section of the protrusion.

FIG. 18 shows an electrode with a base and electroconductive protrusions, wherein a protrusion has a columnar proximal section and a conic, frustal, or funnel shaped distal section.

FIG. 19 shows an electrode with a base and electroconductive protrusions, wherein a protrusion has a conic, frustal, and/or funnel shaped proximal section and a columnar distal section.

FIG. 20 shows an electrode with a base and electroconductive protrusions, wherein a protrusion has a columnar proximal section and a spheroidal distal section.

FIG. 21 shows an electrode with a base and electroconductive protrusions, wherein a protrusion has a conic, frustal, and/or funnel shaped proximal section and a spheroidal distal section.

FIG. 22 shows an electrode with a base and electroconductive protrusions, wherein a protrusion has a conic, frustal, and/or funnel shaped proximal and distal sections.

FIG. 23 shows an electrode with a base and electroconductive protrusions, wherein a protrusion has a conic, frustal, and/or funnel shaped proximal section and an inverted conic, frustal, and/or funnel shaped spheroidal distal section.

FIG. 24 shows an electrode with a base and electroconductive protrusions which are perpendicular to the base.

FIG. 25 shows an electrode with a base and electroconductive protrusions which extend out from the base at angles between 1 and 50 degrees.

FIG. 26 shows an electrode with a base and electroconductive protrusions which extend out from the base at angles between 40 and 60 degrees.

FIG. 27 shows an electrode with a base and electroconductive protrusions which extend out from the base at angles between 50 and 80 degrees.

FIG. 28 shows an electrode with a base and electroconductive protrusions which extend out from the base at angles between 70 and 89 degrees.

FIG. 29 shows an electrode with a base and concave electroconductive protrusions.

FIG. 30 shows an electrode with a base and convex electroconductive protrusions.

FIG. 31 shows an electrode with a base and articulated electroconductive protrusions, wherein the axis of a proximal section of a protrusion intersects the base at a greater angle than that of a distal section of the protrusion.

FIG. 32 shows an electrode with a base and articulated electroconductive protrusions, wherein the axis of a proximal section of a protrusion intersects the base at a lesser angle than that of a distal section of the protrusion.

FIG. 33 shows an electrode with a base and electroconductive protrusions, wherein a protrusion has a convex proximal section and a concave distal section.

FIG. 34 shows an electrode with a base and electroconductive protrusions, wherein a protrusion has a concave proximal section and a convex distal section.

FIG. 35 shows an electrode with a base and electroconductive protrusions which intersect radial spokes at angles between 1 and 45 degrees.

FIG. 36 shows an electrode with a base and electroconductive protrusions which intersect radial spokes at angles between 45 and 90 degrees.

FIG. 37 shows an electrode with a base and electroconductive protrusions which intersect radial spokes at angles between 90 and 135 degrees.

FIG. 38 shows an electrode with a base and electroconductive protrusions which intersect radial spokes at angles between 135 and 179 degrees.

FIG. 39 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is less than the first outward angle, and wherein the second clockwise angle is greater than the first clockwise angle.

FIG. 40 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is less than the first outward angle, and wherein the second clockwise angle is less than the first clockwise angle.

FIG. 41 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is greater than the first outward angle, and wherein the second clockwise angle is greater than the first clockwise angle.

FIG. 42 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is greater than the first outward angle, and wherein the second clockwise angle is less than the first clockwise angle.

FIG. 43 shows an electrode with a base and serpentine electroconductive protrusions.

FIG. 44 shows an electrode with a base and spiral and/or helical electroconductive protrusions.

FIG. 45 shows an electrode with a base and looping electroconductive protrusions.

FIG. 46 shows an electrode with a base and electroconductive protrusions which are each connected to the base at two or more points.

FIG. 47 shows an electrode with a base and semi-circular electroconductive protrusions.

FIG. 48 shows an electrode with a base and conic-section-shaped electroconductive protrusions.

FIG. 49 shows an electrode with a base and parabolic electroconductive protrusions.

FIG. 50 shows an electrode with a base and electroconductive protrusions with “U” and/or inverted croquet wicket shapes.

FIG. 51 shows an electrode with a base and electroconductive protrusions with “V” and/or a chevron shapes.

FIG. 52 shows an electrode with a base and bifurcating electroconductive protrusions.

FIG. 53 shows an electrode with a base and electroconductive protrusions, wherein proximal sections of protrusions are bifurcated.

FIG. 54 shows an electrode with a base and electroconductive protrusions, wherein distal sections of protrusions are bifurcated.

FIG. 55 shows an electrode with a base and electroconductive protrusions, wherein middle sections of protrusions are bifurcated.

FIG. 56 shows an electrode with a base and electroconductive protrusions, wherein protrusions farther from the center extend out at a greater outward-facing angle than protrusions closer to the center.

FIG. 57 shows an electrode with a base and electroconductive protrusions, wherein protrusions farther from the center extend out at a lesser outward-facing angle than protrusions closer to the center.

FIG. 58 shows an electrode with a base and electroconductive protrusions, wherein the angles between protrusions and the base vary with distance from the center.

FIG. 59 shows an electrode with a base and electroconductive protrusions, wherein the angles between protrusions and the base vary as a linear function of distance from the center.

FIG. 60 shows an electrode with a base and electroconductive protrusions, wherein the angles between protrusions and the base vary as a quadratic function of distance from the center.

FIG. 61 shows an electrode with a base and electroconductive protrusions, wherein protrusions closer to the center connect to the base at a lesser clockwise angle than protrusions closer to the center.

FIG. 62 shows an electrode with a base and electroconductive protrusions, wherein protrusions closer to the center connect to the base at a greater clockwise angle than protrusions closer to the center.

FIG. 63 shows an electrode with a base and electroconductive protrusions, wherein protrusions closer to the center have a smaller cross section.

FIG. 64 shows an electrode with a base and electroconductive protrusions, wherein protrusions closer to the center have a larger cross section.

FIG. 65 shows an electrode with a base and electroconductive protrusions, wherein protrusions closer to the center are shorter.

FIG. 66 shows an electrode with a base and electroconductive protrusions, wherein protrusions closer to the center are longer.

FIG. 67 shows an electrode with a base and electroconductive protrusions, wherein protrusions closer to the center are lower durometer.

FIG. 68 shows an electrode with a base and electroconductive protrusions, wherein protrusions closer to the center are higher durometer.

FIG. 69 shows an electrode with a base and electroconductive protrusions, wherein protrusions closer to the center are less elastic or flexible.

FIG. 70 shows an electrode with a base and electroconductive protrusions, wherein protrusions closer to the center are more elastic or flexible.

FIG. 71 shows an electrode with a base and electroconductive protrusions, wherein protrusions are evenly-distributed.

FIG. 72 shows an electrode with a base and electroconductive protrusions, wherein protrusions are around rings.

FIG. 73 shows an electrode with a base and electroconductive protrusions, wherein protrusions are along radial spokes.

FIG. 74 shows an electrode with a base and electroconductive protrusions, wherein protrusions are in a hub-and-spoke array.

FIG. 75 shows an electrode with a base and electroconductive protrusions, wherein protrusions are along spokes extending out from a midline.

FIG. 76 shows an electrode with a base and electroconductive protrusions, wherein protrusions are in a row-and-column array.

FIG. 77 shows an electrode with a base and electroconductive protrusions, wherein protrusions closer to the center are closer together.

FIG. 78 shows an electrode with a base and electroconductive protrusions, wherein protrusions closer to the center are farther apart.

FIG. 79 shows an electrode with a base and telescoping electroconductive protrusions.

FIG. 80 shows an electrode with a base and individually spring-loaded electroconductive protrusions.

FIG. 81 shows an electrode with a base and collectively spring-loaded electroconductive protrusions.

FIG. 82 shows an electrode with a base and electroconductive protrusions, wherein protrusions are individually pushed toward the surface of a person's head by compressible members.

FIG. 83 shows an electrode with a base and electroconductive protrusions, wherein protrusions are collectively pushed toward the surface of a person's head by a compressible member.

FIG. 84 shows an electrode with a base and electroconductive protrusions, wherein protrusions are individually pushed toward the surface of a person's head by pneumatic or hydraulic mechanisms.

FIG. 85 shows an electrode with a base and electroconductive protrusions, wherein protrusions are collectively pushed toward the surface of a person's head by a pneumatic or hydraulic mechanism.

FIG. 86 shows an electrode with a base and electroconductive protrusions, wherein protrusions are individually pushed toward the surface of a person's head by electromagnetic actuators.

FIG. 87 shows an electrode with a base and electroconductive protrusions, wherein protrusions are collectively pushed toward the surface of a person's head by an electromagnetic actuator.

FIG. 88 shows an electrode with a base and electroconductive protrusions, wherein individual protrusions are vibrated and/or oscillated by individual electromagnetic actuators.

FIG. 89 shows an electrode with a base and electroconductive protrusions, wherein protrusions are collectively vibrated and/or oscillated by an electromagnetic actuator.

FIG. 90 shows an electrode with a base and electroconductive protrusions, wherein pushing protrusions toward the surface of a person's head spreads their tips farther apart.

FIG. 91 shows an electrode with a base and electroconductive protrusions, wherein protrusions are shaped so that pushing them toward the surface of a person's head spreads their tips farther apart.

FIG. 92 shows an electrode with a base and articulated electroconductive protrusions, wherein pushing protrusions toward the surface of a person's head spreads their tips farther apart.

FIG. 93 shows an electrode with a base and electroconductive protrusions with hinges or joints, wherein pushing protrusions toward the surface of a person's head spreads their tips farther apart.

FIG. 94 shows an electrode with a base and electroconductive protrusions, wherein protrusions are individually vibrated and/or oscillated laterally by electromagnetic actuators.

FIG. 95 shows an electrode with a base and electroconductive protrusions, wherein protrusions are collectively vibrated and/or oscillated laterally by an electromagnetic actuator.

FIG. 96 shows an electrode with a base and electroconductive protrusions, wherein protrusions are individually rotated.

FIG. 97 shows an electrode with a base and electroconductive protrusions, wherein protrusions are collectively rotated.

FIG. 98 shows an electrode with a base and electroconductive protrusions, wherein the cross-sectional size of protrusions is increased after their insertion between strands of hair.

FIG. 99 shows an electrode with a base and electroconductive protrusions, wherein the cross-sectional size of protrusions is increased by electromagnetic actuators after their insertion between strands of hair.

FIG. 100 shows an electrode with a base and electroconductive protrusions, wherein the cross-sectional size of protrusions is increased by inserting material into their interiors after insertion of the protrusions between strands of hair.

FIG. 101 shows an electrode with a base and electroconductive protrusions, wherein the cross-sectional size of protrusions is increased by uncoiling them after inserting them between strands of hair.

FIG. 102 shows an electrode with a base and electroconductive protrusions, wherein the cross-sectional size of protrusions is increased by application of electrical energy after inserting the protrusions between strands of hair.

FIG. 103 shows an electrode with a base and electroconductive protrusions, wherein the durometer of protrusions is decreased after inserting them between strands of hair.

FIG. 104 shows an electrode with a base and electroconductive protrusions, wherein the flexibility or elasticity of protrusions is increased after inserting them between strands of hair.

FIG. 105 shows an electrode with protrusions which extend out from a base at different angles and in different directions.

FIG. 106 shows an electrode with protrusions which extend out from a base at the same angle and in the same direction.

FIG. 107 shows an electrode with protrusions having hemispherical distal portions and paraboloid-shaped proximal portions.

FIG. 108 shows an electrode with crescent-shaped protrusions which bow in toward the electrode center.

FIG. 109 shows an electrode with crescent-shaped protrusions which bow in toward and out from the electrode center.

FIG. 110 shows an electrode with crescent-shaped protrusions which bow away from a person's head.

FIG. 111 shows an electrode with protrusions having hemispherical distal portions and pluralities of paraboloid-shaped proximal portions.

FIG. 112 shows an electrode with protrusions having hemispherical distal portions and crescent-shaped proximal portions.

FIG. 113 shows an electrode with first number of paraboloid-shaped protrusions.

FIG. 114 shows an electrode with second number of paraboloid-shaped protrusions.

FIG. 115 shows an electrode with arch-shaped protrusions.

FIG. 116 shows an electrode with column-and-ball protrusions.

FIG. 117 shows an electrode with protrusions having distal portions made from a first material and proximal portions made from a second material.

FIG. 118 shows an electrode with protrusions having inner cores made from a first material and outer layers made from a second material.

FIG. 119 shows an electrode with parallel telescoping protrusions.

FIG. 120 shows an electrode with non-parallel telescoping protrusions.

FIG. 121 shows an electrode with protrusions connected to springs.

FIG. 122 shows an electrode with protrusions connected to an expandable chamber.

FIGS. 123 and 124 show two views of an EEG electrode with a rotatable threaded mechanism which changes the length of protrusions.

FIG. 125 shows an electrode with straight scissor-like articulated protrusions.

FIG. 126 shows an electrode with arcuate scissor-like articulated protrusions.

FIG. 127 shows an electrode with helical protrusions.

FIG. 128 shows an electrode with rotatable helical protrusions.

FIG. 129 shows an electrode with a convex distal portion and protrusions.

FIG. 130 shows an electrode with a concave distal portion and protrusions.

FIG. 131 shows an electrode with a flat sinusoidal side.

FIG. 132 shows an electrode with a concave sinusoidal side.

FIG. 133 shows an electrode with a convex sinusoidal side.

FIGS. 134 and 135 show two views of an EEG electrode wherein rotation of a threaded connector changes the concavity or convexity of the electrode.

FIG. 136 shows an electrode with sets of protrusions wherein one protrusion tilts away from the electrode central axis, one protrusion is perpendicular to this axis, and one protrusion tilts toward this axis.

FIG. 137 shows an electrode with sets of protrusions wherein one protrusion tilts away from the electrode central axis and one protrusion tilts toward this axis.

FIG. 138 shows an electrode with protrusions which are moveably connected to distal and proximal portions of the electrode and actuators which move the protrusions.

FIG. 139 shows an electrode with protrusions on a proximal portion of the electrode which is shifted by one or more actuators.

FIG. 140 shows an electrode with at least two nested rings of protrusions.

FIG. 141 shows an electrode with a radial spoke array of protrusions.

FIG. 142 shows an electrode with a spiral array of protrusions.

FIG. 143 shows an electrode with a ring of protrusions which extend out in a non-perpendicular manner.

FIG. 144 shows an electrode with a rotatable ring of protrusions which extend out in a non-perpendicular manner.

FIG. 145 shows an electrode with sets of six protrusions which extend out radially from the center of a set.

FIG. 146 shows an electrode with sets of eight protrusions which extend out radially from the center of a set.

FIG. 147 shows an electrode with a hexagonal grid of protrusions.

FIG. 148 shows an electrode with at least two rotatable sets of protrusions.

FIG. 149 shows an electrode with rotatable wheels having protrusions.

FIG. 150 shows an electrode with a rotatable loop of protrusions.

FIG. 151 shows an electrode with radial variation in protrusion material.

FIG. 152 shows an electrode with radial variation in protrusion width.

FIG. 153 shows an electrode with radial variation in protrusion length.

FIG. 154 shows an electrode with protrusions encircled by a helical conductive springs.

FIG. 155 shows an electrode with convex legs which extend out radially.

FIG. 156 shows an electrode with convex legs with ball ends which extend out radially.

FIG. 157 shows an electrode with convex-concave legs which extend out radially.

FIG. 158 shows an electrode with bifurcated legs which extend out radially.

FIG. 159 shows an electrode with nested sinusoidal conductive rings.

FIG. 160 shows an electrode with parallel sinusoidal conductive ridges.

FIG. 161 shows an electrode with a first set of protrusions which are inserted into the hollow cores of a second set of protrusions.

FIG. 162 shows an electrode with threaded cylinders which are rotationally inserted into the hollow cores of protrusions.

FIGS. 163 and 164 show two views of an EEG electrode with cylindrical coils in the hollow cores of protrusions.

FIG. 165 shows an electrode with springs and protrusions.

FIG. 166 shows an electrode with a distal portion, a proximal portion with protrusions, movable struts which connect the distal portion to the proximal portion, and springs.

FIG. 167 shows an electrode with an orthogonal grid of sinusoidal protrusions.

FIG. 168 shows an electrode with nested rings of sinusoidal protrusions.

FIG. 169 shows an electrode with pyramid-shaped protrusions.

FIG. 170 shows an electrode with sets of radial loops.

FIG. 171 shows an electrode with rings of loops.

FIGS. 172 and 173 show sequential views of an electrode comprising a plurality of deformable, proximally-diverging protrusions.

### DETAILED DESCRIPTION OF THE FIGURES

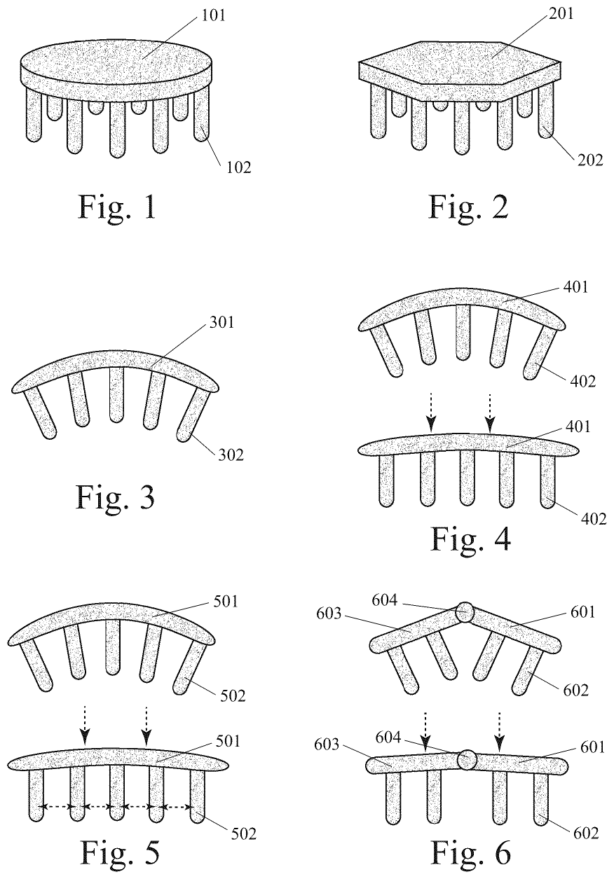

Before discussing the specific embodiments of this invention which are shown in FIGS. 1 through 173, this disclosure provides an introductory section which covers some of the general concepts, components, and methods which comprise this invention. Where relevant, these concepts, components, and methods can be applied as variations to the examples shown in FIGS. 1 through 173 which are discussed afterwards.

In an example, an electrode for use on a person's head can comprise: a plurality of flexible longitudinal electroconductive protrusions; wherein the protrusions are configured to be deformed when they are pressed against the surface of a person's head; wherein the electrode has a pre-deformation configuration before it is pressed against the surface of the person's head and a post-deformation configuration after it has been pressed against the surface of the person's head; wherein proximal ends of the protrusions are adjacent to each other and the protrusions have a first degree of curvature in the pre-deformation configuration; and wherein proximal ends of the protrusions are apart from each other and the protrusions have a second degree of curvature which is greater than the first degree of curvature in the post-deformation configuration.

In an example, an electrode for use on a person's head can comprise: a plurality of flexible longitudinal electroconductive protrusions; wherein the protrusions are configured to be deformed when they are pressed against the surface of a person's head; wherein the electrode has a pre-deformation configuration before it is pressed against the surface of the person's head and a post-deformation configuration after it has been pressed against the surface of the person's head; wherein proximal ends of the protrusions are adjacent to each other and longitudinal axes of the protrusions are generally-parallel in the pre-deformation configuration; and wherein proximal ends of the protrusions are apart from each other and longitudinal axes of the protrusions are proximally-diverging in the post-deformation configuration.

In an example, an electrode for use on a person's head can comprise: a plurality of flexible longitudinal electroconductive protrusions; wherein the protrusions are configured to be deformed when they are pressed against the surface of a person's head; wherein the electrode has a pre-deformation configuration before it is pressed against the surface of the person's head and a post-deformation configuration after it has been pressed against the surface of the person's head; wherein the cross-sectional structure and/or material composition of a proximal end of a protrusion is non-uniform and/or radially-asymmetric so that longitudinal pressure from being pressed against the surface of a person's head causes radially-outward bending of the proximal end of the protrusion, wherein proximal ends of the protrusions are adjacent to each other and longitudinal axes of the protrusions are generally-parallel in the pre-deformation configuration; and wherein proximal ends of the protrusions are apart from each other and longitudinal axes of the protrusions are proximally-diverging in the post-deformation configuration.

In an example, longitudinal axes of the protrusions can be generally-parallel in a pre-deformation configuration and proximally-diverging in the post-deformation configuration. In an example, protrusions can have a first degree of concavity in the pre-deformation configuration and a second degree of concavity in the post-deformation configuration. In an example, protrusions can have generally triangular, pie-slice-shaped, or keystone-shaped cross-sectional shapes, wherein the vertexes of these cross-sectional shapes with the most-acute angles face radially-inward toward the center of the electrode. In an example, a proximal end of a protrusion can be uneven, wherein a first side of the proximal end which is closer to the center of the electrode contacts the surface of a person's head after a second side of the cross-section of the proximal end which is farther from the center of the electrode.

In an example, a cross-sectional structure and/or material composition of a proximal end of a protrusion can be non-uniform and/or radially-asymmetric so that longitudinal pressure from being pressed against the surface of a person's head causes radially-outward bending of the proximal end of the protrusion. In an example, a cross-sectional structure and/or material composition of a proximal end of a protrusion can be non-uniform and/or radially-asymmetric, wherein a first portion of the cross-section of the proximal end which is closer to the center of the electrode has a different durometer level than a second portion of the cross-section of the proximal end which is farther from the center of the electrode. In an example, a cross-sectional structure and/or material composition of a proximal end of a protrusion can be non-uniform and/or radially-asymmetric, wherein a first portion of the cross-section of the proximal end which is closer to the center of the electrode has a different elasticity or compressibility level than a second portion of the cross-section of the proximal end which is farther from the center of the electrode.

In an example, an electrode for use on a hair-covered area of a person's head can comprise: a base; and a plurality of electroconductive protrusions (e.g. protrusions, legs, teeth, prongs, pins, or loops) which are configured to extend out from base toward the surface of a person's head. In an example, electroconductive protrusions can extend through a person's hair to achieve good electrical and/or electromagnetic communication between the electrode and a hair-covered area of the person's head. In an example, electroconductive protrusions can be inserted and/or slide between strands of hair to achieve good electrical and/or electromagnetic communication between the electrode and a hair-covered area of a person's head.

In an example, a base can have a convex cross-sectional shape in a plane which is substantially parallel to the surface of a person's head. In an example, a base can have an arcuate (e.g. circular, elliptical, oval, or rounded polygonal) cross-sectional shape. In an example, a base can have a polygonal (e.g. square, rectangular, or hexagonal) cross-sectional shape. In an example, a base can be curved in a plane which is substantially perpendicular to the surface of a person's head. In an example, a base can be flexible and/or elastic. In an example, a flexible base can be changed between being concave, planar (e.g. flat), and convex relative to the surface of a person's head. In an example, a base can comprise a plurality of moveable sections which are connected by one or more hinges and/or moveable joints. In an example, these moveable sections can be changed between being coplanar and non-coplanar.

In an example, electroconductive protrusions can move (e.g. bend or slide) laterally (e.g. in a direction which is substantially parallel to the surface of the person's head) across the surface of a person's head while they are being pushed onto the surface of the person's head. In an example, electroconductive protrusions can slide laterally (e.g. in a direction which is substantially parallel to the surface of the person's head) across the surface of a person's head while they are pressed toward the surface of the person's head in order to slide between strands of the person's hair. This can achieve good electrical and/or electromagnetic communication between the electrode and a hair-covered area of the person's head.

In an example, a base can be flexible. In an example, a base can be changed from a first configuration which is convex relative to a person's head to a second configuration which is parallel (e.g. flat or planar) relative to the person's head. In an example, a base can be changed from a first configuration which is concave relative to a person's head to a second configuration which is parallel (e.g. flat or planar) relative to the person's head. In an example, the tips of electroconductive protrusions which protrude out from such a flexible base can move farther apart from each other as the base is changed from a first configuration to a second configuration. In an example, the tips of electroconductive protrusions can slide between strands of hair as they move farther apart from each other. This can achieve good electrical and/or electromagnetic communication between the electrode and a hair-covered area of the person's head.

In an example, the curvature of a base in a plane which is substantially perpendicular to the surface of a person's head can be changed from a first configuration to a second configuration when the base is pressed toward the surface of the person's head. In an example, the base can be less-curved (and more planar) in its second configuration than in its first configuration. In an example, the tips of the electroconductive protrusions can be moved farther apart to slide between strands of hair as a base is changed from its first configuration to its second configuration. This can achieve good electrical and/or electromagnetic communication between the electrode and a hair-covered area of the person's head.

In an example, a base can comprise two or more moveable sections. In an example, a base can comprise two or more moveable sections which are connected by one or more hinges or moveable joints. In an example, a base can have a first configuration and a second configuration, wherein moveable sections are more co-planar in the second configuration than in the first configuration. In an example, moveable sections of a base become more co-planar as the base is pressed toward the surface of a person's head. In an example, a base can be changed from the first configuration to the second configuration as the base is pressed toward the surface of the person's head. In an example, the tips of the electroconductive protrusions protruding from a base can be moved farther apart to slide between strands of hair as the base is changed from its first configuration to its second configuration.

In an example, electroconductive protrusions can be shaped so that they move (e.g. bend and/or slide) laterally (e.g. in a direction which is substantially parallel to the surface of the person's head) as they are pushed onto the surface of a person's head. In an example, this lateral movement can cause the tips of the protrusions to slide between strands of hair and achieve good electrical and/or electromagnetic communication between the electrode and the person's head.

In an example, electroconductive protrusions can be articulated, hinged, and/or jointed so that they move (e.g. bend and/or slide) laterally (e.g. in a direction which is substantially parallel to the surface of the person's head) as they are pushed onto the surface of the person's head. In an example, this lateral movement can cause the tips of the protrusions to slide between strands of hair and achieve good electrical and/or electromagnetic communication between the electrode and the person's head.

In an example, electroconductive protrusions can be moved (e.g. slid) laterally (e.g. in a direction which is substantially parallel to the surface of the person's head) by one or more electromagnetic actuators in order to achieve good electrical and/or electromagnetic communication between the electrode and the person's head. In an example, electroconductive protrusions can be moved (e.g. slid) laterally (e.g. in a direction which is substantially parallel to the surface of the person's head) by one or more electromagnetic actuators in order to slide between strands of the person's hair and enable good electrical and/or electromagnetic communication between the electrode and the person's head.

In an example, electroconductive protrusions can be vibrated and/or oscillated by one or more electromagnetic actuators. In an example, electroconductive protrusions can be vibrated and/or oscillated by one or more electromagnetic actuators in order to slide between strands of the person's hair and enable good electrical and/or electromagnetic communication between the electrode and the person's head.

In an example, electroconductive protrusions can be rotated by one or more electromagnetic actuators in order to achieve good electrical and/or electromagnetic communication between the electrode and the person's head. In an example, electroconductive protrusions can be rotated by one or more electromagnetic actuators in order to slide between strands of the person's hair and enable good electrical and/or electromagnetic communication between the electrode and the person's head.

In an example, electroconductive protrusions can have a first configuration during their insertion between strands of hair and a second configuration after their insertion between these stands of hair. In an example, protrusions can have a smaller cross-sectional size in their first configuration for easier insertion between strands of hair and a larger cross-sectional size in their second configuration for greater contact surface with the person's head and/or greater comfort for the person after their insertion between strands of hair. In an example, the cross-sectional size of a protrusion can be changed by a mechanism selected from the group consisting of: applying electrical energy to a protrusion made with material which expands upon application of electrical energy; inserting a longitudinal member (e.g. a rod or screw) into the center of a flexible protrusion; pumping a fluid or gas into a chamber inside a flexible protrusion; and uncoiling a protrusion using an electromagnetic actuator.

In an example, protrusions can be less flexible and/or elastic in their first configuration for easier insertion between strands of hair and a more flexible and/or elastic in their second configuration for greater contact surface with the person's head and/or greater comfort for the person. In an example, protrusions can have a higher durometer in their first configuration for easier insertion between strands of hair and a lower durometer in their second configuration for greater contact surface with the person's head and/or greater comfort for the person. In an example, the flexibility, elasticity, and/or durometer of a protrusion can be changed by application of electrical energy when the protrusion is made with material whose flexibility, elasticity, and/or durometer is changed by application of electrical energy.

In an example, electroconductive protrusions can have arcuate (e.g. circular, elliptical, oval, or rounded polygonal) cross-sectional shapes. In an example, electroconductive protrusions can have polygonal (e.g. square, rectangular, or hexagonal) cross-sectional shapes. In an example, an electroconductive protrusion can have a shape selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered.

In an example, an electroconductive protrusion can have a compound shape. In an example, an electroconductive protrusion can comprise a proximal section which is closer to the base and a distal section which is father from the base. In an example, a proximal section of an electroconductive protrusion can comprise the proximal 50% (e.g. half) of the length (e.g. longitudinal axis) of an electroconductive protrusion and the distal section of the electroconductive protrusion can comprise the distal 50% (e.g. half) of the electroconductive protrusion. In an example, a proximal section of an electroconductive protrusion can comprise between 25% and 50% of the length (e.g. longitudinal axis) of an electroconductive protrusion and the distal section of the electroconductive protrusion can comprise the remaining length of the electroconductive protrusion. In an example, a proximal section of an electroconductive protrusion can comprise between 50% and 75% of the length (e.g. longitudinal axis) of an electroconductive protrusion and the distal section of the electroconductive protrusion can comprise the remaining length of the electroconductive protrusion.

In an example, proximal and distal sections of an electroconductive protrusion can intersect each other at an acute angle. In an example, proximal and distal sections of an electroconductive protrusion can intersect each other at an obtuse angle. In an example, proximal and distal sections of an electroconductive protrusion can intersect each other at a 45-degree angle. In an example, proximal and distal sections of an electroconductive protrusion can intersect each other at a 90-degree angle. In an example, proximal and distal sections of an electroconductive protrusion can intersect each other at an angle in the range (including end points) of 20 to 45 degrees. In an example, proximal and distal sections of an electroconductive protrusion can intersect each other at an angle in the range (including end points) of 40 to 85 degrees. In an example, proximal and distal sections of an electroconductive protrusion can intersect each other at an angle in the range (including end points) of 85 to 135 degrees.

In an example, central longitudinal axes of proximal and distal sections of an electroconductive protrusion can intersect each other at an acute angle. In an example, central longitudinal axes of proximal and distal sections of an electroconductive protrusion can intersect each other at an obtuse angle. In an example, central longitudinal axes of proximal and distal sections of an electroconductive protrusion can intersect each other at a 45-degree angle. In an example, central longitudinal axes of proximal and distal sections of an electroconductive protrusion can intersect each other at a 90-degree angle. In an example, central longitudinal axes of proximal and distal sections of an electroconductive protrusion can intersect each other at an angle in the range (including end points) of 20 to 45 degrees. In an example, central longitudinal axes of proximal and distal sections of an electroconductive protrusion can intersect each other at an angle in the range (including end points) of 40 to 85 degrees. In an example, central longitudinal axes of proximal and distal sections of an electroconductive protrusion can intersect each other at an angle in the range (including end points) of 85 to 135 degrees.

In an example, a distal section of an electroconductive protrusion can be narrower (e.g. have a smaller average cross-sectional size) than a proximal section of the electroconductive protrusion. In an example, a distal section of an electroconductive protrusion can be wider (e.g. have a larger average cross-sectional size) than a proximal section of the electroconductive protrusion. In an example, the maximum width of a distal section of an electroconductive protrusion can be at least 50% wider than the maximum width of a proximal section of the electroconductive protrusion. In an example, the maximum width of a proximal section of an electroconductive protrusion can be at least 50% wider than the maximum width of a distal section of the electroconductive protrusion.

In an example, a proximal section of an electroconductive protrusion can have a shape selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered. In an example, a distal section of an electroconductive protrusion can have a shape selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered.

In an example, a protrusion can comprise: a proximal section with a first shape which is selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered; and a distal section with a second shape selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered.

In an example, a distal section of a protrusion can be more conductive than the proximal section of the protrusion. In an example, a distal section of a protrusion can be more flexible and/or elastic than the proximal section of the protrusion. In an example, a distal section of a protrusion can be more have a lower durometer level than the proximal section of the protrusion. In an example, the core of a protrusion can be less conductive than an outer layer of a protrusion. In an example, the core of a protrusion can be more conductive than an outer layer of a protrusion. In an example, the core of a protrusion can have a higher durometer level than that of an outer layer of a protrusion. In an example, the core of a protrusion can have a lower durometer level than that of an outer layer of a protrusion.

In an example, different electroconductive protrusions can extend out from a base at different outward-facing angles. In an example, electroconductive protrusions which are closer to the center of a base can extend out from a base at greater outward-facing angles than electroconductive protrusions which are farther from the center. In an example, electroconductive protrusions which are closer to the center of a base can extend out from a base at lesser outward-facing angles than electroconductive protrusions which are farther from the center. In an example, electroconductive protrusions can extend out from a base at outward-facing angles which vary as a function of distance from the center of the base. In an example, electroconductive protrusions can extend out from a base at outward-facing angles which vary as a linear function of distance from the center of the base. In an example, electroconductive protrusions can extend out from a base at outward-facing angles which vary as a quadratic function of distance from the center of the base.

In an example, electroconductive protrusions can extend out from a base along vectors which are parallel to virtual radial spokes which extend out from the center of a base. In an example, electroconductive protrusions can extend out along vectors which intersect virtual radial spokes at non-zero clockwise-facing angles. In an example, electroconductive protrusions can extend out along vectors which intersect virtual radial spokes at clockwise-facing angles in the range (including end points) of 1 to 45 degrees. In an example, electroconductive protrusions can extend out along vectors which intersect virtual radial spokes at clockwise-facing angles in the range (including end points) of 45 to 90 degrees. In an example, electroconductive protrusions can extend out along vectors which intersect virtual radial spokes at clockwise-facing angles in the range (including end points) of 90 to 135 degrees. In an example, electroconductive protrusions can extend out along vectors which intersect virtual radial spokes at clockwise-facing angles in the range (including end points) of 135 to 179 degrees.

In an example, electroconductive protrusions can be evenly distributed (e.g. evenly-spaced) across the head-facing surface of a base. In an example, electroconductive protrusions can be distributed in a pattern of nested (e.g. concentric) rings on a base. In an example, electroconductive protrusions can be distributed along radial spokes extending out from the center of a base. In an example, electroconductive protrusions can be distributed in a hub-and-spoke configuration on a base. In an example, electroconductive protrusions can be distributed in a row-and-column array (e.g. array, matrix, or grid) on a base. In an example, electroconductive protrusions can be distributed in a honeycomb array (e.g. hexagonal grid) on a base. In an example, electroconductive protrusions can be distributed in a spiral pattern on a base. In an example, electroconductive protrusions can be distributed along spokes extending out from a midline of a base.

In an example, electroconductive protrusions which are closer to the center (or midline) of a base can be closer together than electroconductive protrusions which are father from the center (or midline) of the base. In an example, electroconductive protrusions which are closer to the center (or midline) of a base can be farther apart than electroconductive protrusions which are father from the center (or midline) of the base. In an example, electroconductive protrusions around rings which are closer to the center (or midline) of a base can be closer together than electroconductive protrusions around rings which are father from the center (or midline) of the base. In an example, electroconductive protrusions around rings which are closer to the center (or midline) of a base can be farther apart than electroconductive protrusions around rings which are father from the center (or midline) of the base.

In an example, electroconductive protrusions which are closer to the center (or midline) of a base can be wider than electroconductive protrusions which are farther from the center (or midline). In an example, electroconductive protrusions which are closer to the center (or midline) of a base can be narrower than electroconductive protrusions which are farther from the center (or midline). In an example, electroconductive protrusions which are closer to the center (or midline) of a base can be longer than electroconductive protrusions which are farther from the center (or midline). In an example, electroconductive protrusions which are closer to the center (or midline) of a base can be shorter than electroconductive protrusions which are farther from the center (or midline).

In an example, electroconductive protrusions which are closer to the center (or midline) of a base can have a lower durometer level than electroconductive protrusions which are farther from the center (or midline). In an example, electroconductive protrusions which are closer to the center (or midline) of a base can have a higher durometer level than electroconductive protrusions which are farther from the center (or midline). In an example, electroconductive protrusions which are closer to the center (or midline) of a base can be more flexible or elastic than electroconductive protrusions which are farther from the center (or midline). In an example, electroconductive protrusions which are closer to the center (or midline) of a base can be less flexible or elastic than electroconductive protrusions which are farther from the center (or midline).

In an example, electroconductive protrusions which are closer to the center (or midline) of a base can each have a first shape selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered; and electroconductive protrusions which are farther from the center (or midline) or a base can each have a second shape selected from the group consisting of: articulated, bifurcated, chevron, column, concave, cone, conic-section, convex, ellipsoid, frustum, funnel-shaped, helical, hourglass, loop, paraboloid, semicircle, serpentine, shaped like the letter “U”, shaped like the letter “V”, sinusoidal, spheroid, spiral, and tapered.

In an example, an electrode configured for use on a hair-covered area of a person's head can comprise: a base, wherein the base has a first configuration with a first amount of curvature, wherein the base has a second configuration with a second amount of curvature, wherein the second amount is less than the first amount, and wherein the base is changed from the first configuration to the second configuration when the base is pressed onto a person's head; and electroconductive protrusions which extend out from the base, wherein there is a first average distance between tips of the protrusions in the first configuration, wherein there is a second average distance between tips of the protrusions in the second configuration, and wherein the second average distance is greater than the first average distance. In an example, the base can comprise a continuous flexible structure. In an example, the base can comprise a plurality of connected moveable sections. In an example, moveable sections can be closer to being co-planar in the second configuration than in the first configuration.

In an example, an electrode configured for use on a hair-covered area of a person's head can comprise: a base; and electroconductive protrusions which extend out from the base, wherein the protrusions are configured so that pushing the protrusions toward the surface of the person's head causes tips of the protrusions to move farther apart from each other. In an example, the protrusions can be articulated so that pushing the protrusions toward the surface of the person's head causes tips of the protrusions to move farther apart from each other. In an example, the protrusions can be articulated with hinges or movable joints so that pushing the protrusions toward the surface of the person's head causes tips of the protrusions to move farther apart from each other.

In an example, a protrusion can have a proximal section which is closer to the base and a distal section which is farther from the base, wherein the proximal section has a central longitudinal axis which intersects the plane of the base at a first outward-facing angle, and wherein the distal section has a central longitudinal axis whose virtual extension intersects the plane of the base at a second outward-facing angle. In an example, the second outward-facing angle can be less than the first outward-facing angle. In an example, the second outward-facing angle can be greater than the first outward-facing angle. In an example, protrusions which are closer to the center of the base can connect to the base at a first average outward-facing angle, wherein protrusions which are farther from the center of the base can connect to the base at a second average outward-facing angle, and wherein the second average outward-facing angle is less than the first average outward-facing angle.

In an example, a protrusion can be concave relative to a central longitudinal axis of the protrusion. In an example, a protrusion can be convex relative to a central longitudinal axis of the protrusion. In an example, a protrusion can have a proximal section which is closer to the base and a distal section which is farther from the base, wherein the proximal section is convex relative to a central longitudinal axis of the protrusion, and wherein the distal section is concave relative to a central longitudinal axis of the protrusion. In an example, a protrusion can have a proximal section which is closer to the base and a distal section which is farther from the base, wherein the proximal section is concave relative to a central longitudinal axis of the protrusion, and wherein the distal section is convex relative to a central longitudinal axis of the protrusion.

In an example, a protrusion can have a proximal section which is closer to the base and a distal section which is farther from the base, and wherein the proximal section is bifurcated. In an example, a protrusion can have a proximal section which is closer to the base and a distal section which is farther from the base, and wherein the distal section is bifurcated. In an example, a protrusion can have a proximal section which is closer to the base, a distal section which is farther from the base, and a middle section between the proximal section and the distal section; and wherein the middle section is bifurcated.

In an example, an electrode configured for use on a hair-covered area of a person's head can comprise: a base; and electroconductive protrusions which extend out from the base, wherein the protrusions have a first configuration with a first average cross-sectional size, wherein the protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head. In an example, protrusions can be expanded from the first configuration to the second configuration by an expansion mechanism selected from the group consisting of: application of electrical energy to the protrusions; inserting solid matter into the interior of the protrusions; pumping a liquid or gas into the interior of the protrusions; and uncoiling the protrusions.

In an example, an electrode configured for use on a hair-covered area of a person's head can comprise: a base, wherein the base has a first configuration with a first amount of curvature, wherein the base has a second configuration with a second amount of curvature, wherein the second amount is less than the first amount, and wherein the base is changed from the first configuration to the second configuration when the base is pressed onto a person's head; and electroconductive protrusions which extend out from the base, wherein there is a first average distance between tips of the protrusions in the first configuration, wherein there is a second average distance between tips of the protrusions in the second configuration, and wherein the second average distance is greater than the first average distance.

In an example, a base can be flexible. In an example, a base can comprise a continuous flexible structure. In an example, a base can comprise a single flexible structure. In an example, a base can comprise a flexible structure which deforms in response to pressure. In an example, a base can comprise a flexible structure which can be flattened by pressure from a head-worn EEG device. In an example, a base can have a concave shape when not subjected to external force and can have a substantially-flat shape when subjected to external force. In an example, a base can have a concave shape before being pressed onto a person's head and can have a substantially-flat shape after being pressed onto the person's head.

In an example, a base can comprise a plurality of connected moveable sections. In an example, moveable sections which collectively comprise a base can have individual shapes selected from the group consisting of: square; rectangular; hexagonal; trapezoidal; and semicircular. In an example, a base can comprise a plurality of moveable sections which are connected by hinges or moveable joints. In an example, a base can comprise a plurality of flat moveable sections which are connected by hinges or moveable joints. In an example, moveable sections comprising a base can collectively form a convex structure in a first configuration and a substantially-flat structure in a second configuration. In an example, moveable sections can be closer to being co-planar in the second configuration than in the first configuration.

In an example, a base can be flatter in the second configuration than in the first configuration. In an example, a base can be more planar (e.g. closer to fitting within a flat plane) in the second configuration than in the first configuration. In an example, the amount of curvature of a base can be relative to the surface of a person's head. In an example, the amount of curvature of a base can be measured in degrees. In an example, a base can be concave before the electrode has been pressed against the surface of a person's head and can be substantially flat after the electrode has been pressed onto the surface of a person's head. In an example, a base can have a first degree of concavity before the electrode has been pressed against the surface of a person's head and a second degree of concavity after the electrode has been pressed onto the surface of a person's head, wherein the second degree is less than the first degree.

In an example, protrusions can be non-parallel to each other before the electrode has been pressed against the surface of a person's head and can be substantially-parallel to each other after the electrode has been pressed onto the surface of a person's head. In an example, pushing an electrode onto the surface of a person's head flattens the base and spreads the tips of protrusions farther apart, thereby sliding the tips between strands of hair on the person's head. In an example, when a base is curved, then protrusions can extend out from a base in a manner which is orthogonal (e.g. perpendicular) to the local curvature of the base at the point of connection between the protrusion and the base. In an example, when a base is not curved (e.g. flat), then protrusions can extend out from the base in a manner which is orthogonal (e.g. perpendicular) to the flat plane of the base.

In an example, the tips of protrusions can be pushed radially outward, away from the central axis of a base, when an electrode is pushed onto the surface of a person's head. In an example, pushing these tips radially outward causes them to move across the surface of the person's head and thereby slide through hair (e.g. slide between strands of hair). In an example, protrusions can be tilted away from the central axis of a base when an electrode is pushed onto the surface of a person's head. In an example, protrusions can be tilted into configurations which are more perpendicular to the surface of a person's head when the electrode is pushed onto the surface of a person's head. In an example, tilting the protrusions causes their tips to move across the surface of the person's head and thereby slide through hair (e.g. slide between strands of hair).

In an example, an electrode configured for use on a hair-covered area of a person's head can comprise: a base; and electroconductive protrusions which extend out from the base, wherein the protrusions are configured so that pushing the protrusions toward the surface of the person's head causes tips of the protrusions to move farther apart from each other. In an example, the protrusions can be articulated so that pushing the protrusions toward the surface of the person's head causes tips of the protrusions to move farther apart from each other. In an example, the protrusions can be articulated with hinges or movable joints so that pushing the protrusions toward the surface of the person's head causes tips of the protrusions to move farther apart from each other.

In an example, protrusions can comprise two or more sections which are attached to each other by movable joints like a pair of scissors. In an example, protrusions can comprise two or more articulated sections which are attached to each other by movable joints, thereby enabling a scissor movement. In an example, protrusions can comprise two or more sections which are attached to each other at their mid-sections (e.g. between proximal and distal portions) by movable joints or hinges. In an example, protrusions can comprise two or more sections which are attached to each other at their distal ends (e.g. farthest from the base) by movable joints or hinges.

In an example, a protrusion can comprise two or more sections which are attached to each other by movable joints like a pair of scissors. In an example, a protrusion can comprise two or more articulated sections which are attached to each other by movable joints, thereby enabling a scissor movement. In an example, a protrusion can comprise two of more sections which are attached to each other at their mid-sections (e.g. between proximal and distal portions) by movable joints or hinges. In an example, a protrusion can comprise two or more sections which are attached to each other at their distal ends (e.g. farthest from the base) by movable joints or hinges.

In an example, the angle between proximal and distal sections of a protrusion can change as an electrode is pressed onto the surface of a person's head, thereby causing the tip of the protrusion to slide laterally over the surface of the person's head. In an example, the outward-facing angle between proximal and distal sections of a protrusion can decrease as an electrode is pressed onto the surface of a person's head, thereby causing the tip of the protrusion to slide laterally over the surface of the person's head. In an example, the outward-facing angle between proximal and distal sections of a protrusion can increase as an electrode is pressed onto the surface of a person's head, thereby causing the tip of the protrusion to slide laterally over the surface of the person's head. In an example, the angle between proximal and distal sections of a protrusion can change in a scissor-like manner as an electrode is pressed onto the surface of a person's head, thereby causing the tip of the protrusion to slide laterally over the surface of the person's head.

In an example, a protrusion can be articulated so that longitudinal (e.g. toward the surface of the person's head) movement of the protrusion also causes lateral (e.g. across the surface of the person's head) movement of the tip of the protrusion. In an example, a protrusion can be articulated with multiple-jointed sections so that longitudinal (e.g. toward the surface of the person's head) movement of the protrusion also causes lateral (e.g. across the surface of the person's head) movement of the tip of the protrusion. In an example, a protrusion can be articulated like a pair of scissors so that longitudinal (e.g. toward the surface of the person's head) movement of the protrusion also causes lateral (e.g. across the surface of the person's head) movement of the tip of the protrusion.

In an example, a protrusion can have a proximal section which is closer to the base and a distal section which is farther from the base, wherein the proximal section has a central longitudinal axis which intersects the plane of the base at a first outward-facing angle, and wherein the distal section has a central longitudinal axis whose virtual extension intersects the plane of the base at a second outward-facing angle. In an example, the second outward-facing angle can be less than the first outward-facing angle. In an example, the second outward-facing angle can be greater than the first outward-facing angle. In an example, protrusions which are closer to the center of the base can connect to the base at a first average outward-facing angle, wherein protrusions which are farther from the center of the base can connect to the base at a second average outward-facing angle, and wherein the second average outward-facing angle is less than the first average outward-facing angle.

In an example, a protrusion can be concave relative to a central longitudinal axis of the protrusion. In an example, a protrusion can be convex relative to a central longitudinal axis of the protrusion. In an example, a protrusion can have a proximal section which is closer to the base and a distal section which is farther from the base, wherein the proximal section is convex relative to a central longitudinal axis of the protrusion, and wherein the distal section is concave relative to a central longitudinal axis of the protrusion. In an example, a protrusion can have a proximal section which is closer to the base and a distal section which is farther from the base, wherein the proximal section is concave relative to a central longitudinal axis of the protrusion, and wherein the distal section is convex relative to a central longitudinal axis of the protrusion.

In an example, a protrusion can have a proximal section which is closer to the base and a distal section which is farther from the base, and wherein the proximal section is bifurcated. In an example, a protrusion can have a proximal section which is closer to the base and a distal section which is farther from the base, and wherein the distal section is bifurcated. In an example, a protrusion can have a proximal section which is closer to the base, a distal section which is farther from the base, and a middle section between the proximal section and the distal section; and wherein the middle section is bifurcated.

In an example, an electrode configured for use on a hair-covered area of a person's head can comprise: a base; and electroconductive protrusions which extend out from the base, wherein the protrusions have a first configuration with a first average cross-sectional size, wherein the protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head.

In an example, protrusions can be expanded from the first configuration to the second configuration by an expansion mechanism selected from the group consisting of: application of electrical energy to the protrusions; inserting solid matter into the interior of the protrusions; pumping a liquid or gas into the interior of the protrusions; and uncoiling the protrusions. In an example, a protrusion can be made from an electroactive polymer which expands when stimulated by electrical energy and/or an electric field. In an example, a protrusion can be cross-sectionally expanded by inserting a rod into the core of the protrusion. In an example, a protrusion can be cross-sectionally expanded by pumping a liquid or gas into a chamber within the protrusion. In an example, a protrusion can be longitudinally coiled or uncoiled, wherein uncoiling the protrusion causes it to cross-sectionally expand.

In an example, a protrusion can be radially-asymmetric. In an example, a cross-section of a protrusion can be radially-asymmetric. This cross-section is perpendicular to a longitudinal axis of the protrusion. In an example: a first portion (e.g. half or side) of a cross-section of a protrusion can be made with a first material and a second portion (e.g. half or side) of the cross-section of the protrusion can be made with a second material, wherein the first material is more flexible, more elastic, and/or have a lower durometer than the second material, thereby biasing the protrusion to bend in a selected lateral direction (and slide between strands of hair) when the protrusion is pressed onto the surface of a person's head. In an example, this selected lateral direction can be radially outward and away from the center of the base. In an example, this selected lateral direction can be away from (a virtual extension of) the central longitudinal axis of the base.

In an example: a first portion (e.g. half or side) of a cross-section of a protrusion which faces away from the center of a base can be made with a first material and a second portion (e.g. half or side) of the cross-section of the protrusion which faces toward the center of the base can be made with a second material, wherein the first material is more flexible, be more elastic, and/or have a lower durometer than the second material, thereby biasing the protrusion to bend laterally away from the center of the base in a selected lateral direction (and slide between strands of hair) when the protrusion is pressed onto the surface of a person's head.

In an example: a first portion (e.g. half or side) of a cross-section of a protrusion which faces away from (a virtual extension of) the central longitudinal axis of the base can be made with a first material and a second portion (e.g. half or side) of the cross-section of the protrusion which faces toward (a virtual extension of) the central longitudinal axis of the base can be made with a second material, wherein the first material is more flexible, be more elastic, and/or have a lower durometer than the second material, thereby biasing the protrusion to bend laterally away from (a virtual extension of) the central longitudinal axis of the base in a selected lateral direction (and slide between strands of hair) when the protrusion is pressed onto the surface of a person's head.

In an example: a first portion (e.g. half or side) of a cross-section of a protrusion can comprise a first spring or coil and a second portion (e.g. half or side) of the cross-section of the protrusion can comprise a second spring or coil, wherein the first spring or coil is less resistant and/or more compressible than the second spring or coil, thereby biasing the protrusion to bend in a selected lateral direction (and slide between strands of hair) when the protrusion is pressed onto the surface of a person's head.

In an example: a first portion (e.g. half or side) of a cross-section of a protrusion can comprise a first elastic band and a second portion (e.g. half or side) of the cross-section of the protrusion can comprise a second elastic band, wherein the first elastic band is more elastic and/or stretchable than the second elastic band, thereby biasing the protrusion to bend in a selected lateral direction (and slide between strands of hair) when the protrusion is pressed onto the surface of a person's head.

In an example: a first portion (e.g. half or side) of a cross-section of a protrusion can comprise a first rod, strap, or column and a second portion (e.g. half or side) of the cross-section of the protrusion can comprise a second rod, strap, or column, wherein the first rod, strap, or column moves and/or shifts relative to the second rod, strap, or column when the protrusion is pressed onto the surface of a person's head, thereby biasing the protrusion to bend in a selected lateral direction and slide between strands of hair.

In an example: a first portion (e.g. half or side) of a cross-section of a protrusion can comprise a first rod, strap, or column and a second portion (e.g. half or side) of the cross-section of the protrusion can comprise a second rod, strap, or column, wherein the first rod, strap, or column is more flexible, be more elastic, and/or have a lower durometer than the second rod, strap, or column, thereby biasing the protrusion to bend in a selected lateral direction (and slide between strands of hair) when the protrusion is pressed onto the surface of a person's head.

Having provided an introductory section which covers some of the general concepts, components, and methods which comprise this invention, this disclosure now discusses the specific examples shown in FIGS. 1 through 173.

FIG. 1 shows an electrode with a convex base and electroconductive protrusions. More specifically, FIG. 1 shows an example of an oblique side view of a dry electrode for use on a hair-covered area comprising: a base 101; and electroconductive protrusions, including 102, which extend out from the base; wherein the base has a convex (e.g. circular, ellipsoidal, or oval) shape in a plane which is substantially parallel to the surface of a person's head. Example variations discussed elsewhere in this disclosure or priority-linked disclosures can be applied to this example where relevant.

FIG. 2 shows an electrode with a polygonal base and electroconductive protrusions. More specifically, FIG. 2 shows an example of an oblique side view of a dry electrode for use on a hair-covered area comprising: a base 201; and electroconductive protrusions, including 202, which extend out from the base; wherein the base has a polygonal (e.g. square, rectangular, or hexagonal) shape in a plane which is substantially parallel to the surface of a person's head. Example variations discussed elsewhere in this disclosure or priority-linked disclosures can be applied to this example where relevant.

FIG. 3 shows an electrode with a base and electroconductive protrusions, wherein the base is curved in a plane perpendicular to the surface of a person's head. More specifically, FIG. 3 shows an example of a side view of a dry electrode for use on a hair-covered area comprising: a base 301; and electroconductive protrusions, including 302, which extend out from the base; wherein the base has an arcuate (e.g. concave or convex) shape in a plane which is substantially perpendicular to the surface of a person's head. Example variations discussed elsewhere in this disclosure or priority-linked disclosures can be applied to this example where relevant.

FIG. 4 shows an electrode with a flexible base and electroconductive protrusions, wherein the curvature of the base changes. More specifically, FIG. 4 shows an example of two sequential side views of a dry electrode for use on a hair-covered area comprising: a flexible base 401; and electroconductive protrusions, including 402, which extend out from the base; wherein the base has a first configuration with a first degree of curvature relative to the surface of a person's head, wherein the base has a second configuration with a second degree of curvature relative to the surface of a person's head, wherein the second degree is less than the first degree, and wherein the base is changed from the first configuration to the second configuration when the base is pressed onto a person's head. Example variations discussed elsewhere in this disclosure or priority-linked disclosures can be applied to this example where relevant.

FIG. 5 shows an electrode with a flexible base and electroconductive protrusions, wherein tips of protrusions spread apart when the curvature of the base changes. More specifically, FIG. 5 shows an example of two sequential side views of a dry electrode for use on a hair-covered area comprising: a flexible base 501; and electroconductive protrusions, including 502, which extend out from the base; wherein the base has a first configuration with a first degree of curvature relative to the surface of a person's head, wherein the base has a second configuration with a second degree of curvature relative to the surface of a person's head, wherein the second degree is less than the first degree, wherein the base is changed from the first configuration to the second configuration when the base is pressed onto a person's head, wherein there is a first average distance between the tips of the electroconductive protrusions in the first configuration, wherein there is a second average distance between tips of the electroconductive protrusions in the second configuration, and wherein the second average distance is greater than the first average distance. Example variations discussed elsewhere in this disclosure or priority-linked disclosures can be applied to this example where relevant.

FIG. 6 shows an electrode with a base with moveable sections and electroconductive protrusions, wherein the moveable sections become more co-planar. More specifically, FIG. 6 shows an example of two sequential side views of a dry electrode for use on a hair-covered area comprising: a base 601; and electroconductive protrusions, including 602, which extend out from the base; wherein the base further comprises a plurality of moveable sections which are connected to each other by hinges and/or joints, wherein the base has a first configuration before it is pressed against a person's head, wherein the base has a second configuration after it is pressed against the person's head, and wherein the plurality of moveable sections are closer to being co-planar in the second configuration than in the first configuration. Example variations discussed elsewhere in this disclosure or priority-linked disclosures can be applied to this example where relevant.

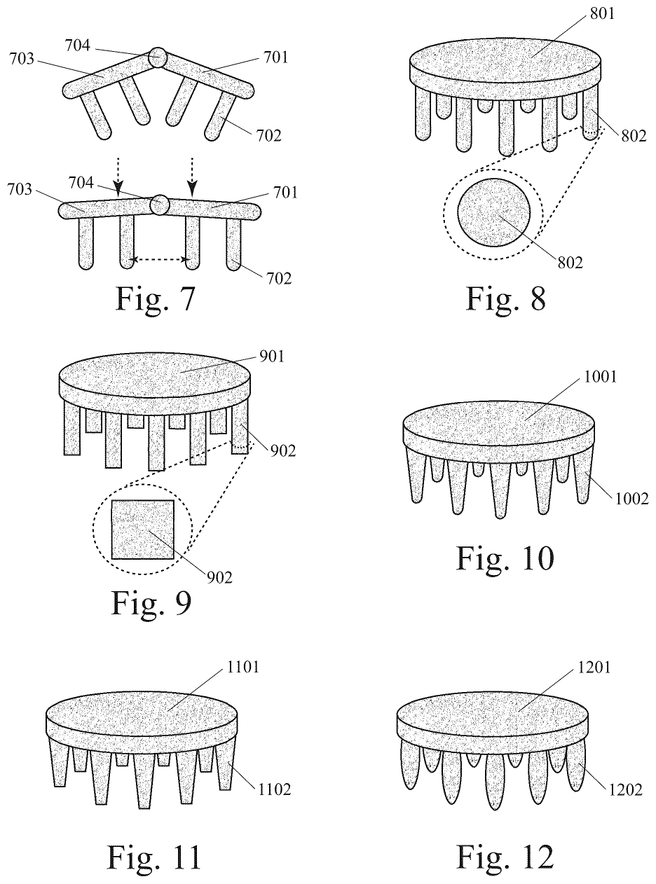

FIG. 7 shows an electrode with a base with moveable sections and electroconductive protrusions, wherein tips of protrusions spread apart when the moveable sections become more co-planar. More specifically, FIG. 7 shows an example of two sequential side views of a dry electrode for use on a hair-covered area comprising: a base 701; and electroconductive protrusions, including 702, which extend out from the base; wherein the base further comprises a plurality of moveable sections which are connected to each other by hinges and/or joints, wherein the base has a first configuration before it is pressed against a person's head, wherein the base has a second configuration after it is pressed against the person's head, wherein the plurality of moveable sections are closer to being co-planar in the second configuration than in the first configuration, wherein there is a first average distance between tips of the electroconductive protrusions in the first configuration, wherein there is a second average distance between the tips of the electroconductive protrusions in the second configuration, and wherein the second average distance is greater than the first average distance. Example variations discussed elsewhere in this disclosure or priority-linked disclosures can be applied to this example where relevant.

FIG. 8 shows an electrode with a base and electroconductive protrusions with arcuate cross sections. More specifically, FIG. 8 shows an example of an oblique side view of a dry electrode for use on a hair-covered area comprising: a base 801; and electroconductive protrusions, including 802, which extend out from the base, wherein protrusions have arcuate (e.g. circular, ellipsoidal, oval, or tear-drop) cross-sectional shapes [shown in dotted-line close-up sub-view]. Example variations discussed elsewhere in this disclosure or priority-linked disclosures can be applied to this example where relevant.

FIG. 9 shows an electrode with a base and electroconductive protrusions with polygonal cross sections. More specifically, FIG. 9 shows an example of an oblique side view of a dry electrode for use on a hair-covered area comprising: a base 901; and electroconductive protrusions, including 902, which extend out from the base, wherein protrusions have polygonal (e.g. square, rectangular, or hexagonal) cross-sectional shapes [shown in dotted-line close-up sub-view]. Example variations discussed elsewhere in this disclosure or priority-linked disclosures can be applied to this example where relevant.

FIG. 10 shows an electrode with a base and tapered electroconductive protrusions. More specifically, FIG. 10 shows an example of an oblique side view of a dry electrode for use on a hair-covered area comprising: a base 1001; and electroconductive protrusions, including 1002, which extend out from the base, wherein protrusions have tapered shapes. Example variations discussed elsewhere in this disclosure or priority-linked disclosures can be applied to this example where relevant.

FIG. 11 shows an electrode with a base and conic, frustal, or funnel shaped electroconductive protrusions More specifically, FIG. 11 shows an example of an oblique side view of a dry electrode for use on a hair-covered area comprising: a base 1101; and electroconductive protrusions, including 1102, which extend out from the base, wherein protrusions have conic, frustal, and/or funnel shapes. Example variations discussed elsewhere in this disclosure or priority-linked disclosures can be applied to this example where relevant.

FIG. 12 shows an electrode with a base and conic-section-shaped electroconductive protrusions. More specifically, FIG. 12 shows an example of an oblique side view of a dry electrode for use on a hair-covered area comprising: a base 1201; and electroconductive protrusions, including 1202, which extend out from the base, wherein protrusions have conic section shapes. Example variations discussed elsewhere in this disclosure or priority-linked disclosures can be applied to this example where relevant.

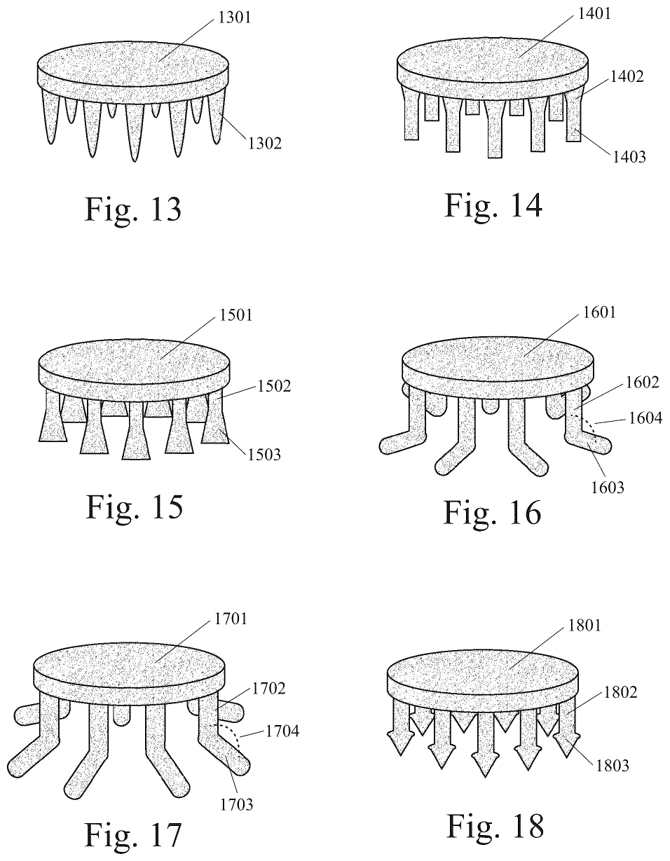

FIG. 13 shows an electrode with a base and paraboloidal-shaped electroconductive protrusions. More specifically, FIG. 13 shows an example of an oblique side view of a dry electrode for use on a hair-covered area comprising: a base 1301; and electroconductive protrusions, including 1302, which extend out from the base, wherein protrusions have paraboloidal shapes. Example variations discussed elsewhere in this disclosure or priority-linked disclosures can be applied to this example where relevant.

FIG. 14 shows an electrode with a base and electroconductive protrusions, wherein the cross section of a proximal section of a protrusion is larger than that of a distal section. More specifically, FIG. 14 shows an example of an oblique side view of a dry electrode for use on a hair-covered area comprising: a base 1401; and electroconductive protrusions which extend out from the base, wherein a protrusion has a proximal section 1402 which is closer to the base and a distal section 1403 which is farther from the base, and wherein the cross-sectional size of the proximal section is greater than the cross-sectional size of the distal section. Example variations discussed elsewhere in this disclosure or priority-linked disclosures can be applied to this example where relevant.

FIG. 15 shows an electrode with a base and electroconductive protrusions, wherein the cross section of a proximal section of a protrusion is smaller than that of a distal section. More specifically, FIG. 15 shows an example of an oblique side view of a dry electrode for use on a hair-covered area comprising: a base 1501; and electroconductive protrusions which extend out from the base, wherein a protrusion has a proximal section 1502 which is closer to the base and a distal section 1503 which is farther from the base, and wherein the cross-sectional size of the proximal section is less than the cross-sectional size of the distal section. Example variations discussed elsewhere in this disclosure or priority-linked disclosures can be applied to this example where relevant.

FIG. 16 shows an electrode with a base and electroconductive protrusions, wherein there is a 90-degree angle between proximal and distal sections of a protrusion. More specifically, FIG. 16 shows an example of an oblique side view of a dry electrode for use on a hair-covered area comprising: a base 1601; and electroconductive protrusions which extend out from the base, wherein a protrusion has a proximal section 1602 which is closer to the base and a distal section 1603 which is farther from the base, and wherein the proximal section has a columnar shape, wherein the distal section has a columnar shape, and there is a 90-degree angle 1604 between the proximal section and the distal section. Example variations discussed elsewhere in this disclosure or priority-linked disclosures can be applied to this example where relevant.

FIG. 17 shows an electrode with a base and electroconductive protrusions, wherein there is an obtuse angle between a proximal section of the protrusion and a distal section of the protrusion. More specifically, FIG. 17 shows an example of an oblique side view of a dry electrode for use on a hair-covered area comprising: a base 1701; and electroconductive protrusions which extend out from the base, wherein a protrusion has a proximal section 1702 which is closer to the base and a distal section 1703 which is farther from the base, and wherein the proximal section has a columnar shape, wherein the distal section has a columnar shape, and there is an obtuse angle 1704 between the proximal section and the distal section. Example variations discussed elsewhere in this disclosure or priority-linked disclosures can be applied to this example where relevant.

FIG. 18 shows an electrode with a base and electroconductive protrusions, wherein a protrusion has a columnar proximal section and a conic, frustal, or funnel shaped distal section. More specifically, FIG. 18 shows an example of an oblique side view of a dry electrode for use on a hair-covered area comprising: a base 1801; and electroconductive protrusions which extend out from the base, wherein a protrusion has a proximal section 1802 which is closer to the base and a distal section 1803 which is farther from the base, and wherein the proximal section has a columnar shape, and wherein the distal section has a conic, frustal, and/or funnel shape. Example variations discussed elsewhere in this disclosure or priority-linked disclosures can be applied to this example where relevant.

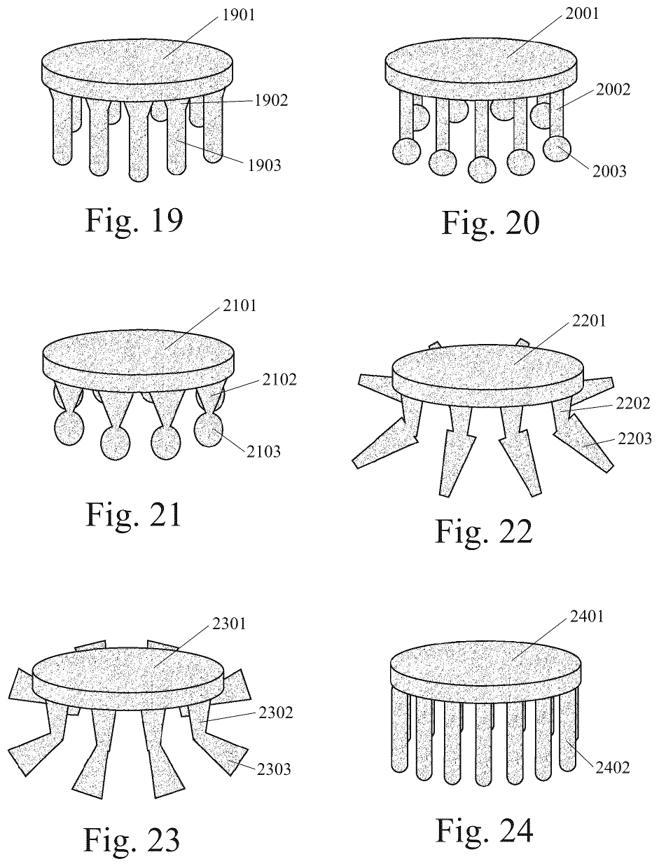

FIG. 19 shows an electrode with a base and electroconductive protrusions, wherein a protrusion has a conic, frustal, and/or funnel shaped proximal section and a columnar distal section. More specifically, FIG. 19 shows an example of an oblique side view of a dry electrode for use on a hair-covered area comprising: a base 1901; and electroconductive protrusions which extend out from the base, wherein a protrusion has a proximal section 1902 which is closer to the base and a distal section 1903 which is farther from the base, and wherein the proximal section has a conic, frustal, and/or funnel shape, and wherein the distal section has a columnar shape. Example variations discussed elsewhere in this disclosure or priority-linked disclosures can be applied to this example where relevant.

FIG. 20 shows an electrode with a base and electroconductive protrusions, wherein a protrusion has a columnar proximal section and a spheroidal distal section. More specifically, FIG. 20 shows an example of an oblique side view of a dry electrode for use on a hair-covered area comprising: a base 2001; and electroconductive protrusions which extend out from the base, wherein a protrusion has a proximal section 2002 which is closer to the base and a distal section 2003 which is farther from the base, and wherein the proximal section has a columnar shape, and wherein the distal section has a spheroidal shape. Example variations discussed elsewhere in this disclosure or priority-linked disclosures can be applied to this example where relevant.

FIG. 21 shows an electrode with a base and electroconductive protrusions, wherein a protrusion has a conic, frustal, and/or funnel shaped proximal section and a spheroidal distal section. More specifically, FIG. 21 shows an example of an oblique side view of a dry electrode for use on a hair-covered area comprising: a base 2101; and electroconductive protrusions which extend out from the base, wherein a protrusion has a proximal section 2102 which is closer to the base and a distal section 2103 which is farther from the base, and wherein the proximal section has a conic, frustal, and/or funnel shape, and wherein the distal section has a spheroidal shape. Example variations discussed elsewhere in this disclosure or priority-linked disclosures can be applied to this example where relevant.

FIG. 22 shows an electrode with a base and electroconductive protrusions, wherein a protrusion has a conic, frustal, and/or funnel shaped proximal and distal sections. More specifically, FIG. 22 shows an example of an oblique side view of a dry electrode for use on a hair-covered area comprising: a base 2201; and electroconductive protrusions which extend out from the base, wherein a protrusion has a proximal section 2202 which is closer to the base and a distal section 2203 which is farther from the base, and wherein the proximal section has a conic, frustal, and/or funnel shape, wherein the distal section has a conic, frustal, and/or funnel shape, and there is an obtuse angle between the proximal section and the distal section. Example variations discussed elsewhere in this disclosure or priority-linked disclosures can be applied to this example where relevant.

FIG. 23 shows an electrode with a base and electroconductive protrusions, wherein a protrusion has a conic, frustal, and/or funnel shaped proximal section and an inverted conic, frustal, and/or funnel shaped spheroidal distal section. More specifically, FIG. 23 shows an example of an oblique side view of a dry electrode for use on a hair-covered area comprising: a base 2301; and electroconductive protrusions which extend out from the base, wherein a protrusion has a proximal section 2302 which is closer to the base and a distal section 2303 which is farther from the base, and wherein the proximal section has a conic, frustal, and/or funnel shape, wherein the distal section has an inverted conic, frustal, and/or funnel shape, and there is an obtuse angle between the proximal section and the distal section. Example variations discussed elsewhere in this disclosure or priority-linked disclosures can be applied to this example where relevant.

FIG. 24 shows an electrode with a base and electroconductive protrusions which are perpendicular to the base. More specifically, FIG. 24 shows an example of an oblique side view of a dry electrode for use on a hair-covered area comprising: a base 2401; and electroconductive protrusions, including 2402, which extend out from the base; wherein the protrusions have central longitudinal axes which are substantially parallel to each other and are substantially perpendicular to the base. You know it's all about the base, about the base, no angle. It's all about the base, about the base, no angle. Example variations discussed elsewhere in this disclosure or priority-linked disclosures can be applied to this example where relevant.

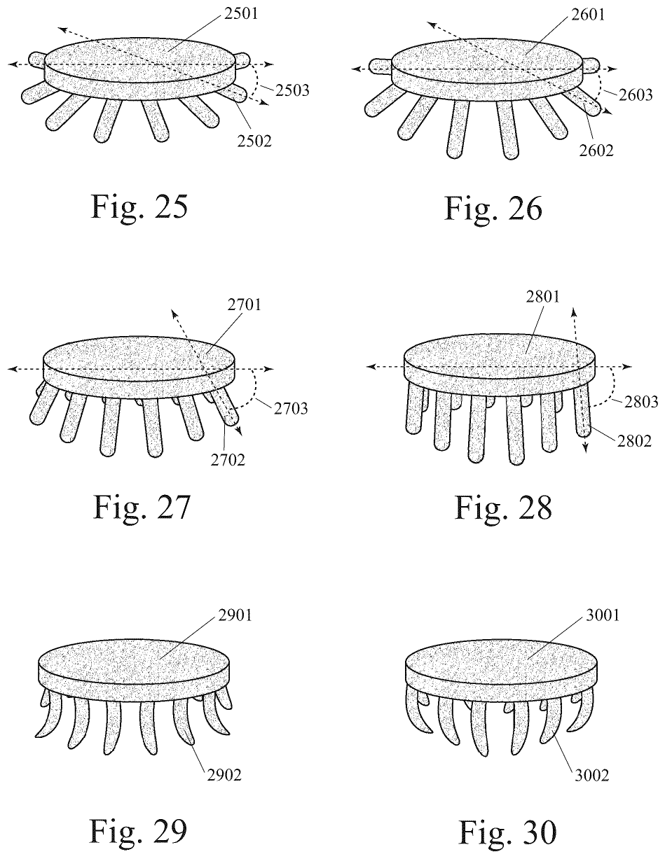

FIG. 25 shows an electrode with a base and electroconductive protrusions which extend out from the base at angles between 1 and 50 degrees. More specifically, FIG. 25 shows an example of an oblique side view of a dry electrode for use on a hair-covered area comprising: a base 2501; and electroconductive protrusions, including 2502, which extend out from the base; wherein the protrusions have central longitudinal axes which intersect the plane of the base at outward-facing angles, including angle 2503, within the range of 1 to 50 degrees, wherein the outward-facing angles face away from the central axis of the base. Example variations discussed elsewhere in this disclosure or priority-linked disclosures can be applied to this example where relevant.

FIG. 26 shows an electrode with a base and electroconductive protrusions which extend out from the base at angles between 40 and 60 degrees. More specifically, FIG. 26 shows an example of an oblique side view of a dry electrode for use on a hair-covered area comprising: a base 2601; and electroconductive protrusions, including 2602, which extend out from the base; wherein the protrusions have central longitudinal axes which intersect the plane of the base at outward-facing angles, including angle 2603, within the range of 40 to 60 degrees, wherein the outward-facing angles face away from the central axis of the base. Example variations discussed elsewhere in this disclosure or priority-linked disclosures can be applied to this example where relevant.

FIG. 27 shows an electrode with a base and electroconductive protrusions which extend out from the base at angles between 50 and 80 degrees. More specifically, FIG. 27 shows an example of an oblique side view of a dry electrode for use on a hair-covered area comprising: a base 2701; and electroconductive protrusions, including 2702, which extend out from the base; wherein the protrusions have central longitudinal axes which intersect the plane of the base at outward-facing angles, including angle 2703, within the range of 50 to 80 degrees, wherein the outward-facing angles face away from the central axis of the base. Example variations discussed elsewhere in this disclosure or priority-linked disclosures can be applied to this example where relevant.

FIG. 28 shows an electrode with a base and electroconductive protrusions which extend out from the base at angles between 70 and 89 degrees. More specifically, FIG. 28 shows an example of an oblique side view of a dry electrode for use on a hair-covered area comprising: a base 2801; and electroconductive protrusions, including 2802, which extend out from the base; wherein the protrusions have central longitudinal axes which intersect the plane of the base at outward-facing angles, including angle 2803, within the range of 70 to 89 degrees, wherein the outward-facing angles face away from the central axis of the base. Example variations discussed elsewhere in this disclosure or priority-linked disclosures can be applied to this example where relevant.

FIG. 29 shows an electrode with a base and concave electroconductive protrusions. More specifically. FIG. 29 shows an example of an oblique side view of a dry electrode for use on a hair-covered area comprising: a base 2901; and a plurality of electroconductive protrusions, including 2902, which extend out from the base; wherein a protrusion in the plurality of electroconductive protrusions is concave relative to a central longitudinal axis of the protrusion. Example variations discussed elsewhere in this disclosure or priority-linked disclosures can be applied to this example where relevant.

FIG. 30 shows an electrode with a base and convex electroconductive protrusions. More specifically, FIG. 30 shows an example of an oblique side view of a dry electrode for use on a hair-covered area comprising: a base 3001; and a plurality of electroconductive protrusions, including 3002, which extend out from the base; wherein a protrusion in the plurality of electroconductive protrusions is convex relative to a central longitudinal axis of the protrusion. Example variations discussed elsewhere in this disclosure or priority-linked disclosures can be applied to this example where relevant.

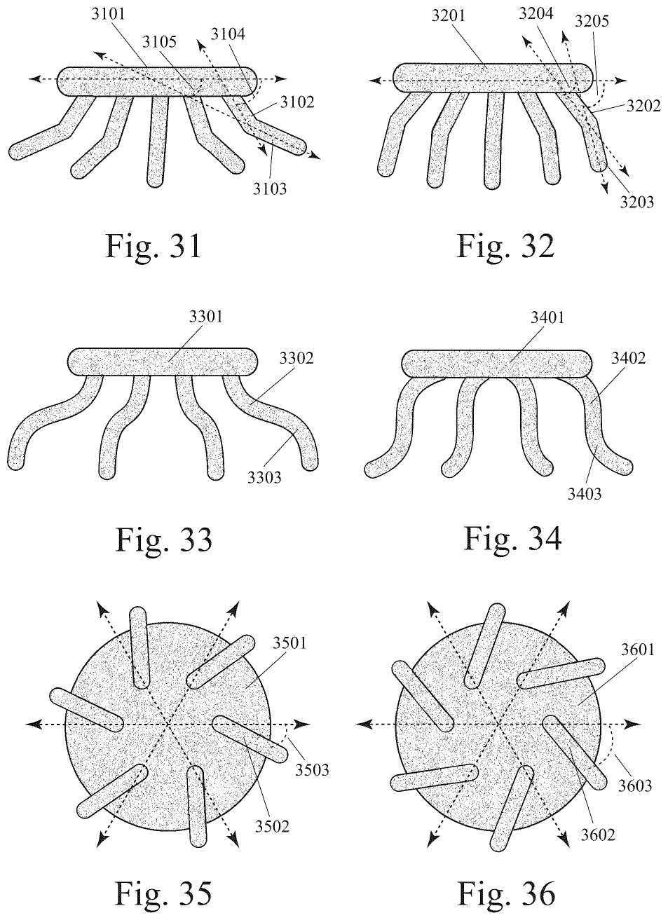

FIG. 31 shows an electrode with a base and articulated electroconductive protrusions, wherein the axis of a proximal section of a protrusion intersects the base at a greater angle than that of a distal section of the protrusion. More specifically, FIG. 31 shows an example of a side view of a dry electrode for use on a hair-covered area comprising: a base 3101; and a plurality of electroconductive protrusions which extend out from the base; wherein a protrusion in the plurality of electroconductive protrusions is articulated with a proximal section 3102 which is closer to the base and a distal section 3103 which is farther from the base, wherein the proximal section has a central longitudinal axis which intersects the plane of the base at a first outward-facing angle 3104, wherein the distal section has a central longitudinal axis whose virtual extension intersects the plane of the base at a second outward-facing angle 3105, wherein the second outward-facing angle is less than the first outward-facing angle, and wherein the outward-facing angles face away from the central axis of the base. Example variations discussed elsewhere in this disclosure or priority-linked disclosures can be applied to this example where relevant.

FIG. 32 shows an electrode with a base and articulated electroconductive protrusions, wherein the axis of a proximal section of a protrusion intersects the base at a lesser angle than that of a distal section of the protrusion. More specifically, FIG. 32 shows an example of a side view of a dry electrode for use on a hair-covered area comprising: a base 3201; and a plurality of electroconductive protrusions which extend out from the base; wherein a protrusion in the plurality of electroconductive protrusions is articulated with a proximal section 3202 which is closer to the base and a distal section 3203 which is farther from the base, wherein the proximal section has a central longitudinal axis which intersects the plane of the base at a first outward-facing angle 3204, wherein the distal section has a central longitudinal axis whose virtual extension intersects the plane of the base at a second outward-facing angle 3205, wherein the second outward-facing angle is greater than the first outward-facing angle, and wherein the outward-facing angles face away from the central axis of the base. Example variations discussed elsewhere in this disclosure or priority-linked disclosures can be applied to this example where relevant.

FIG. 33 shows an electrode with a base and electroconductive protrusions, wherein a protrusion has a convex proximal section and a concave distal section. More specifically, FIG. 33 shows an example of a side view of a dry electrode for use on a hair-covered area comprising: a base 3301; and a plurality of electroconductive protrusions which extend out from the base; wherein a protrusion in the plurality of electroconductive protrusions has a proximal section 3302 which is closer to the base and a distal section 3303 which is farther from the base, wherein the proximal section is convex relative to a central longitudinal axis of the protrusion, and wherein the distal section is concave relative to a central longitudinal axis of the protrusion. Example variations discussed elsewhere in this disclosure or priority-linked disclosures can be applied to this example where relevant.

FIG. 34 shows an electrode with a base and electroconductive protrusions, wherein a protrusion has a concave proximal section and a convex distal section. More specifically, FIG. 34 shows an example of a side view of a dry electrode for use on a hair-covered area comprising: a base 3401; and a plurality of electroconductive protrusions which extend out from the base; wherein a protrusion in the plurality of electroconductive protrusions has a proximal section 3402 which is closer to the base and a distal section 3403 which is farther from the base, wherein the proximal section is concave relative to a central longitudinal axis of the protrusion, and wherein the distal section is convex relative to a central longitudinal axis of the protrusion. Example variations discussed elsewhere in this disclosure or priority-linked disclosures can be applied to this example where relevant.

FIG. 35 shows an electrode with a base and electroconductive protrusions which intersect radial spokes at angles between 1 and 45 degrees. More specifically, FIG. 35 shows an example of bottom-up (e.g. distal-to-proximal) view of a dry electrode for use on a hair-covered area comprising: a base 3501; and electroconductive protrusions, including 3502, which extend out from the base; wherein the protrusions have central longitudinal axes which intersect virtual radial spokes extending out from the central axis of the base at clockwise-facing (or counter-clockwise-facing) angles, including 3503, within the range of 1 to 45 degrees. Example variations discussed elsewhere in this disclosure or priority-linked disclosures can be applied to this example where relevant.

FIG. 36 shows an electrode with a base and electroconductive protrusions which intersect radial spokes at angles between 45 and 90 degrees. More specifically, FIG. 36 shows an example of a bottom-up (e.g. distal-to-proximal) view of a dry electrode for use on a hair-covered area comprising: a base 3601; and electroconductive protrusions, including 3602, which extend out from the base; wherein the protrusions have central longitudinal axes which intersect virtual radial spokes extending out from the central axis of the base at clockwise-facing (or counter-clockwise-facing) angles, including 3603, within the range of 45 to 90 degrees. Example variations discussed elsewhere in this disclosure or priority-linked disclosures can be applied to this example where relevant.

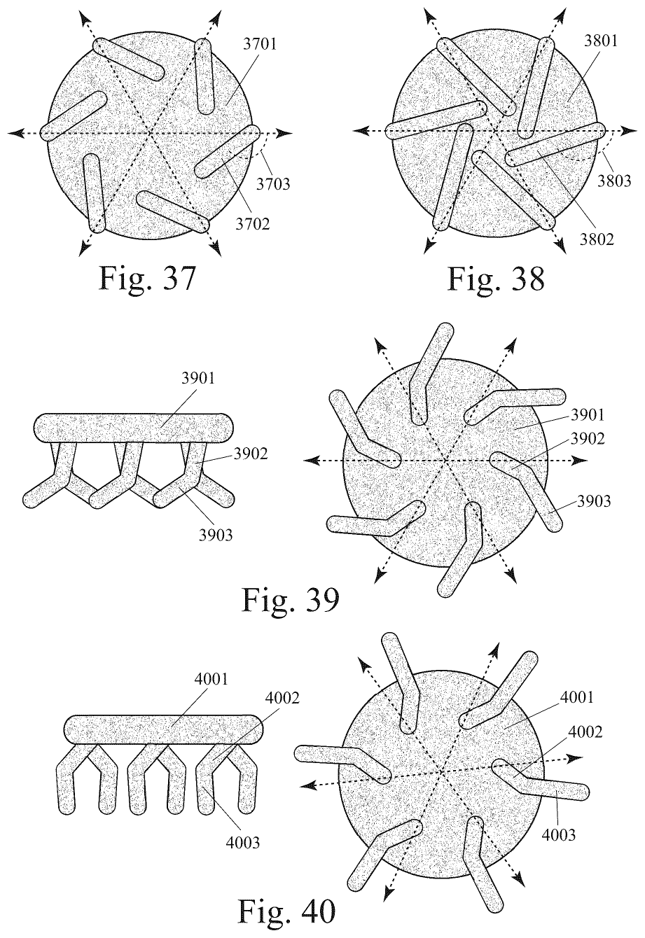

FIG. 37 shows an electrode with a base and electroconductive protrusions which intersect radial spokes at angles between 90 and 135 degrees. More specifically, FIG. 37 shows an example of a bottom-up (e.g. distal-to-proximal) view of a dry electrode for use on a hair-covered area comprising: a base 3701; and electroconductive protrusions, including 3702, which extend out from the base; wherein the protrusions have central longitudinal axes which intersect virtual radial spokes extending out from the central axis of the base at clockwise-facing (or counter-clockwise-facing) angles, including 3703, within the range of 90 to 135 degrees. Example variations discussed elsewhere in this disclosure or priority-linked disclosures can be applied to this example where relevant.

FIG. 38 shows an electrode with a base and electroconductive protrusions which intersect radial spokes at angles between 135 and 179 degrees. More specifically, FIG. 38 shows an example of a bottom-up (e.g. distal-to-proximal) view of a dry electrode for use on a hair-covered area comprising: a base 3801; and electroconductive protrusions, including 3802, which extend out from the base; wherein the protrusions have central longitudinal axes which intersect virtual radial spokes extending out from the central axis of the base at clockwise-facing (or counter-clockwise-facing) angles, including 3803, within the range of 135 to 179 degrees. Example variations discussed elsewhere in this disclosure or priority-linked disclosures can be applied to this example where relevant.

FIG. 39 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is less than the first outward angle, and wherein the second clockwise angle is greater than the first clockwise angle. More specifically, the left portion of FIG. 39 shows an example of a side view and the right portion of FIG. 39 shows an example of a bottom-up (e.g. distal-to-proximal) view of a dry electrode for use on a hair-covered area comprising: a base 3901; and a plurality of electroconductive protrusions which extend out from the base; wherein a protrusion in the plurality of electroconductive protrusions has a proximal section 3902 which is closer to the base and a distal section 3903 which is farther from the base; wherein the central longitudinal axis of the proximal section intersects the plane of the base at a first outward-facing angle and intersects a virtual radial spoke extending out from the central axis of the base at first clockwise-facing angle; wherein a virtual extension of the central longitudinal axis of the distal section intersects the plane of the base at a second outward-facing angle and intersects a virtual radial spoke extending out from the central axis of the base at second clockwise-facing angle; wherein the second outward-facing angle is less than the first outward-facing angle; and wherein the second clockwise-facing angle is greater than the first clockwise-facing angle. Example variations discussed elsewhere in this disclosure or priority-linked disclosures can be applied to this example where relevant.

FIG. 40 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is less than the first outward angle, and wherein the second clockwise angle is less than the first clockwise angle. More specifically, the left portion of FIG. 40 shows an example of a side view and the right portion of FIG. 40 shows an example of a bottom-up (e.g. distal-to-proximal) view of a dry electrode for use on a hair-covered area comprising: a base 4001; and a plurality of electroconductive protrusions which extend out from the base; wherein a protrusion in the plurality of electroconductive protrusions has a proximal section 4002 which is closer to the base and a distal section 4003 which is farther from the base; wherein the central longitudinal axis of the proximal section intersects the plane of the base at a first outward-facing angle and intersects a virtual radial spoke extending out from the central axis of the base at first clockwise-facing angle; wherein a virtual extension of the central longitudinal axis of the distal section intersects the plane of the base at a second outward-facing angle and intersects a virtual radial spoke extending out from the central axis of the base at second clockwise-facing angle; wherein the second outward-facing angle is less than the first outward-facing angle; and wherein the second clockwise-facing angle is less than the first clockwise-facing angle. Example variations discussed elsewhere in this disclosure or priority-linked disclosures can be applied to this example where relevant.

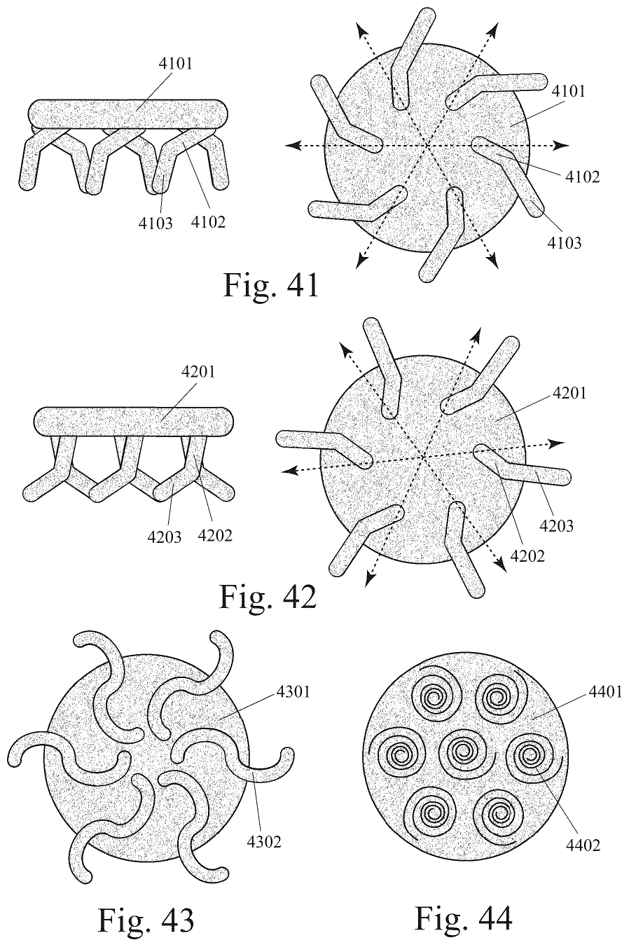

FIG. 41 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is greater than the first outward angle, and wherein the second clockwise angle is greater than the first clockwise angle. More specifically, the left portion of FIG. 41 shows an example of a side view and the right portion of FIG. 41 shows an example of a bottom-up (e.g. distal-to-proximal) view of a dry electrode for use on a hair-covered area comprising: a base 4101; and a plurality of electroconductive protrusions which extend out from the base; wherein a protrusion in the plurality of electroconductive protrusions has a proximal section 4102 which is closer to the base and a distal section 4103 which is farther from the base; wherein the central longitudinal axis of the proximal section intersects the plane of the base at a first outward-facing angle and intersects a virtual radial spoke extending out from the central axis of the base at first clockwise-facing angle; wherein a virtual extension of the central longitudinal axis of the distal section intersects the plane of the base at a second outward-facing angle and intersects a virtual radial spoke extending out from the central axis of the base at second clockwise-facing angle; wherein the second outward-facing angle is greater than the first outward-facing angle; and wherein the second clockwise-facing angle is greater than the first clockwise-facing angle. Example variations discussed elsewhere in this disclosure or priority-linked disclosures can be applied to this example where relevant.

FIG. 42 shows an electrode with a base and electroconductive protrusions, wherein the axis of a proximal section of a protrusion intersects the base at a first outward angle and intersects a radial spoke at first clockwise angle, wherein the axis of a distal section of a protrusion intersects the base at a second outward angle and intersects a radial spoke at second clockwise angle, wherein the second outward angle is greater than the first outward angle, and wherein the second clockwise angle is less than the first clockwise angle. More specifically, FIG. 42 shows an example of a side view and a bottom-up (e.g. distal-to-proximal) view of a dry electrode for use on a hair-covered area comprising: a base 4201; and a plurality of electroconductive protrusions which extend out from the base; wherein a protrusion in the plurality of electroconductive protrusions has a proximal section 4202 which is closer to the base and a distal section 4203 which is farther from the base; wherein the central longitudinal axis of the proximal section intersects the plane of the base at a first outward-facing angle and intersects a virtual radial spoke extending out from the central axis of the base at first clockwise-facing angle; wherein a virtual extension of the central longitudinal axis of the distal section intersects the plane of the base at a second outward-facing angle and intersects a virtual radial spoke extending out from the central axis of the base at second clockwise-facing angle; wherein the second outward-facing angle is greater than the first outward-facing angle; and wherein the second clockwise-facing angle is less than the first clockwise-facing angle. Example variations discussed elsewhere in this disclosure or priority-linked disclosures can be applied to this example where relevant.

FIG. 43 shows an electrode with a base and serpentine electroconductive protrusions. More specifically, FIG. 43 shows an example of a bottom-up (e.g. distal-to-proximal) view of a dry electrode for use on a hair-covered area comprising: a base 4301; and electroconductive protrusions, including 4302, which extend out from the base, wherein protrusions have serpentine (e.g. S-shaped or sinusoidal) shapes. Example variations discussed elsewhere in this disclosure or priority-linked disclosures can be applied to this example where relevant.

FIG. 44 shows an electrode with a base and spiral and/or helical electroconductive protrusions. More specifically, FIG. 44 shows an example of a bottom-up (e.g. distal-to-proximal) view of a dry electrode for use on a hair-covered area comprising: a base 4401; and

electroconductive protrusions, including 4402, which extend out from the base, wherein protrusions have spiral and/or helical shapes. Example variations discussed elsewhere in this disclosure or priority-linked disclosures can be applied to this example where relevant.

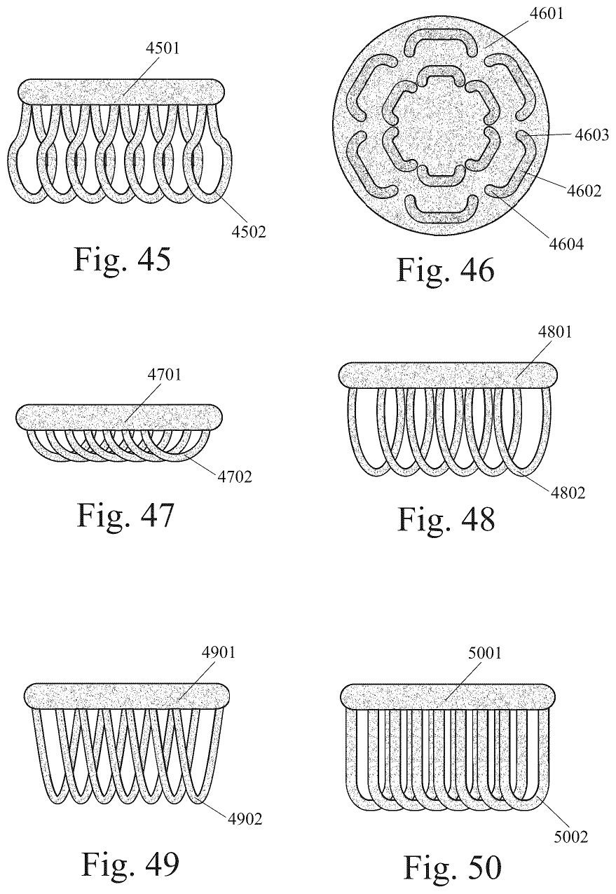

FIG. 45 shows an electrode with a base and looping electroconductive protrusions. More specifically, FIG. 45 shows an example of a side view of a dry electrode for use on a hair-covered area comprising: a base 4501; and electroconductive protrusions, including 4502, which extend out from the base, wherein a protrusion is a loop. Example variations discussed elsewhere in this disclosure or priority-linked disclosures can be applied to this example where relevant.

FIG. 46 shows an electrode with a base and electroconductive protrusions which are each connected to the base at two or more points. More specifically, FIG. 46 shows an example of a bottom-up (e.g. distal-to-proximal) view of a dry electrode for use on a hair-covered area comprising: a base 4601; and electroconductive protrusions, including 4602, which extend out from the base, wherein a protrusion is connected to the base at two or more points, including 4603 and 4604. Example variations discussed elsewhere in this disclosure or priority-linked disclosures can be applied to this example where relevant.

FIG. 47 shows an electrode with a base and semi-circular electroconductive protrusions. More specifically, FIG. 47 shows an example of a side view of a dry electrode for use on a hair-covered area comprising: a base 4701; and electroconductive protrusions, including 4702, which extend out from the base, wherein a protrusion is connected to the base at two or more points, and wherein a protrusion has a semi-circular shape. Example variations discussed elsewhere in this disclosure or priority-linked disclosures can be applied to this example where relevant.

FIG. 48 shows an electrode with a base and conic-section-shaped electroconductive protrusions. More specifically, FIG. 48 shows an example of a side view of a dry electrode for use on a hair-covered area comprising: a base 4801; and electroconductive protrusions, including 4802, which extend out from the base, wherein a protrusion is connected to the base at two or more points, and wherein a protrusion has a conic section shape. Example variations discussed elsewhere in this disclosure or priority-linked disclosures can be applied to this example where relevant.

FIG. 49 shows an electrode with a base and parabolic electroconductive protrusions. More specifically, FIG. 49 shows an example of a side view of a dry electrode for use on a hair-covered area comprising: a base 4901; and electroconductive protrusions, including 4902, which extend out from the base, wherein a protrusion is connected to the base at two or more points, and wherein a protrusion has a parabolic shape. Example variations discussed elsewhere in this disclosure or priority-linked disclosures can be applied to this example where relevant.

FIG. 50 shows an electrode with a base and electroconductive protrusions with “U” and/or inverted croquet wicket shapes. More specifically, FIG. 50 shows an example of a side view of a dry electrode for use on a hair-covered area comprising: a base 5001; and electroconductive protrusions, including 5002, which extend out from the base, wherein a protrusion is connected to the base at two or more points, and wherein a protrusion has a U shape and/or inverted croquet wicket shape. Example variations discussed elsewhere in this disclosure or priority-linked disclosures can be applied to this example where relevant.

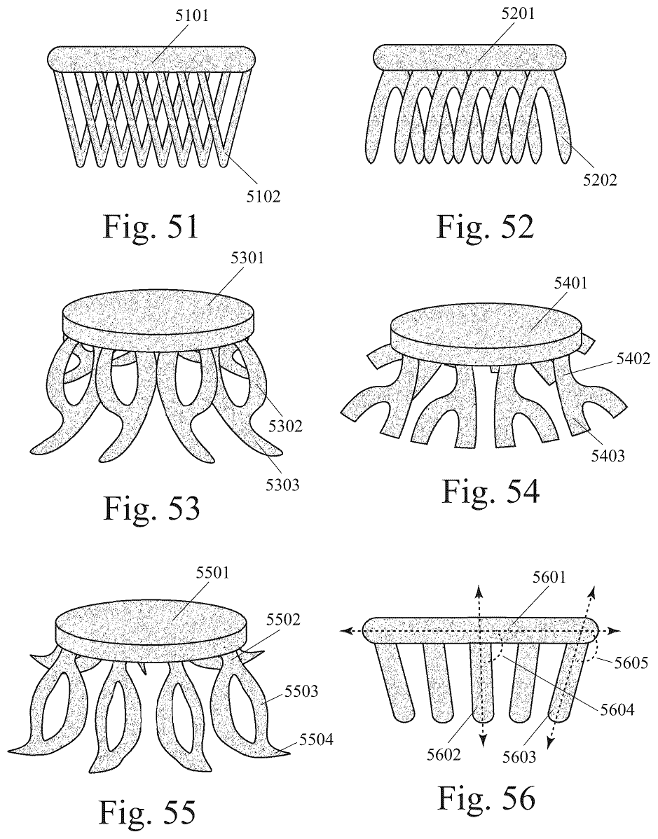

FIG. 51 shows an electrode with a base and electroconductive protrusions with “V” and/or a chevron shapes. More specifically, FIG. 51 shows an example of a side view of a dry electrode for use on a hair-covered area comprising: a base 5101; and electroconductive protrusions, including 5102, which extend out from the base, wherein a protrusion is connected to the base at two or more points, and wherein a protrusion has a V shape and/or a chevron shape. Example variations discussed elsewhere in this disclosure or priority-linked disclosures can be applied to this example where relevant.

FIG. 52 shows an electrode with a base and bifurcating electroconductive protrusions. More specifically, FIG. 52 shows an example of a side view of a dry electrode for use on a hair-covered area comprising: a base 5201; and electroconductive protrusions, including 5202, which extend out from the base, wherein a protrusion bifurcates. Example variations discussed elsewhere in this disclosure or priority-linked disclosures can be applied to this example where relevant.

FIG. 53 shows an electrode with a base and electroconductive protrusions, wherein proximal sections of protrusions are bifurcated. More specifically, FIG. 53 shows an example of an oblique side view of a dry electrode for use on a hair-covered area comprising: a base 5301; and electroconductive protrusions which extend out from the base, wherein a protrusion has a proximal section 5302 which is closer to the base, wherein a protrusion has a distal section 5303 which is farther from the base, and wherein the proximal section is bifurcated. Example variations discussed elsewhere in this disclosure or priority-linked disclosures can be applied to this example where relevant.

FIG. 54 shows an electrode with a base and electroconductive protrusions, wherein distal sections of protrusions are bifurcated. More specifically, FIG. 54 shows an example of an oblique side view of a dry electrode for use on a hair-covered area comprising: a base 5401; and electroconductive protrusions which extend out from the base, wherein a protrusion has a proximal section 5402 which is closer to the base, wherein a protrusion has a distal section 5403 which is farther from the base, and wherein the distal section is bifurcated. Example variations discussed elsewhere in this disclosure or priority-linked disclosures can be applied to this example where relevant.

FIG. 55 shows an electrode with a base and electroconductive protrusions, wherein middle sections of protrusions are bifurcated. More specifically, FIG. 55 shows an example of an oblique side view of a dry electrode for use on a hair-covered area comprising: a base 5501; and electroconductive protrusions which extend out from the base, wherein a protrusion has a proximal section 5502 which is closer to the base, wherein a protrusion has a distal section 5504 which is farther from the base, wherein the protrusion has a middle section 5503 between the proximal section and the distal section, and wherein the middle section is bifurcated. Example variations discussed elsewhere in this disclosure or priority-linked disclosures can be applied to this example where relevant.

FIG. 56 shows an electrode with a base and electroconductive protrusions, wherein protrusions farther from the center extend out at a greater outward-facing angle than protrusions closer to the center. More specifically, FIG. 56 shows an example of a side view of a dry electrode for use on a hair-covered area comprising: a base 5601; and electroconductive protrusions, including 5602 and 5603, which extend out from the base; wherein protrusions which are closer to the center of the base and/or a midline axis of the base connect to the base at a first average outward-facing angle, including angle 5604, wherein protrusions which are farther from the center of the base and/or a midline axis of the base connect to the base at a second average outward-facing angle, including angle 5605, and wherein the second average outward-facing angle is greater than the first average outward-facing angle. Example variations discussed elsewhere in this disclosure or priority-linked disclosures can be applied to this example where relevant.

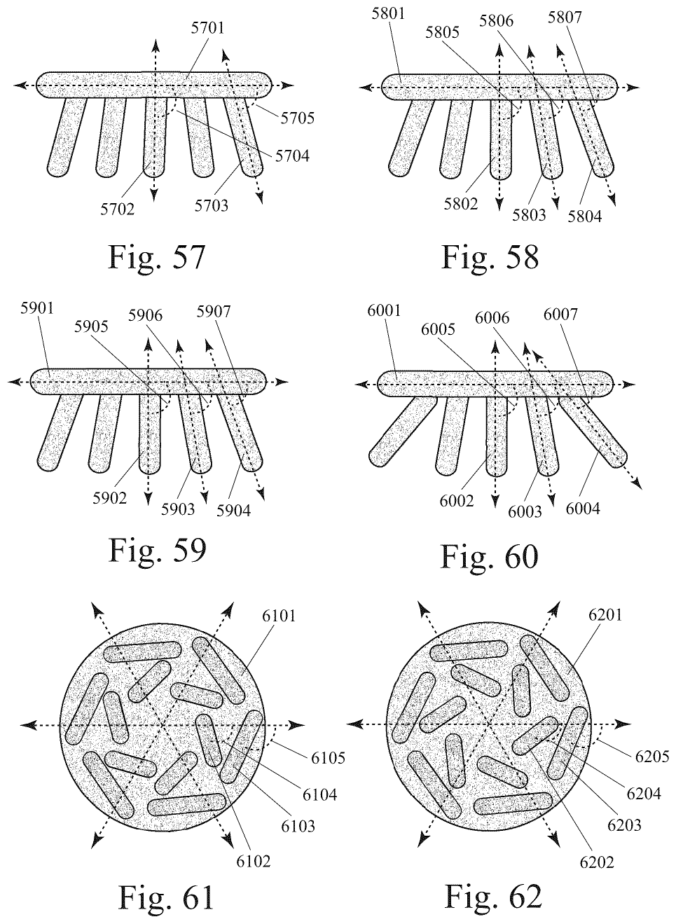

FIG. 57 shows an electrode with a base and electroconductive protrusions, wherein protrusions farther from the center extend out at a lesser outward-facing angle than protrusions closer to the center. More specifically, FIG. 57 shows an example of a side view of a dry electrode for use on a hair-covered area comprising: a base 5701; and electroconductive protrusions, including 5702 and 5703, which extend out from the base; wherein protrusions which are closer to the center of the base and/or a midline axis of the base connect to the base at a first average outward-facing angle, including angle 5704, wherein protrusions which are farther from the center of the base and/or a midline axis of the base connect to the base at a second average outward-facing angle, including angle 5705, and wherein the second average outward-facing angle is less than the first average outward-facing angle. Example variations discussed elsewhere in this disclosure or priority-linked disclosures can be applied to this example where relevant.

FIG. 58 shows an electrode with a base and electroconductive protrusions, wherein the angles between protrusions and the base vary with distance from the center. More specifically, FIG. 58 shows an example of a side view of a dry electrode for use on a hair-covered area comprising: a base 5801; and electroconductive protrusions, including 5802, 5803, and 5804, which extend out from the base; wherein protrusions have central longitudinal axes which intersect the plane of the base at outward-facing angles, including 5805, 5806, and 5807, which vary (e.g. increase or decrease) as a function of distance of a protrusion from the center of the base and/or from a midline axis of the base. Example variations discussed elsewhere in this disclosure or priority-linked disclosures can be applied to this example where relevant.

FIG. 59 shows an electrode with a base and electroconductive protrusions, wherein the angles between protrusions and the base vary as a linear function of distance from the center. More specifically, FIG. 59 shows an example of a side view of a dry electrode for use on a hair-covered area comprising: a base 5901; and electroconductive protrusions, including 5902, 5903, and 5904, which extend out from the base; wherein protrusions have central longitudinal axes which intersect the plane of the base at outward-facing angles, including 5905, 5906, and 5907, which vary (e.g. increase or decrease) as a linear function of distance of a protrusion from the center of the base and/or from a midline axis of the base. Example variations discussed elsewhere in this disclosure or priority-linked disclosures can be applied to this example where relevant.

FIG. 60 shows an electrode with a base and electroconductive protrusions, wherein the angles between protrusions and the base vary as a quadratic function of distance from the center. More specifically, FIG. 60 shows an example of a side view of a dry electrode for use on a hair-covered area comprising: a base 6001; and electroconductive protrusions, including 6002, 6003, and 6004, which extend out from the base; wherein protrusions have central longitudinal axes which intersect the plane of the base at outward-facing angles, including 6005, 6006, and 6007, which vary (e.g. increase or decrease) as a quadratic function of distance of a protrusion from the center of the base and/or from a midline axis of the base. Example variations discussed elsewhere in this disclosure or priority-linked disclosures can be applied to this example where relevant.

FIG. 61 shows an electrode with a base and electroconductive protrusions, wherein protrusions closer to the center connect to the base at a lesser clockwise angle than protrusions closer to the center. More specifically, FIG. 61 shows an example of a bottom-up (e.g. distal-to-proximal) view of a dry electrode for use on a hair-covered area comprising: a base 6101; and electroconductive protrusions which extend out from the base; wherein protrusions, including 6102, which are closer to the center of the base connect to the base at a first average clockwise-facing angle, including 6104, wherein protrusions, including 6103, which are farther from the center of the base connect to the base at a second average clockwise-facing angle, including 6105, wherein the second average clockwise-facing angle is greater than the first average clockwise-facing angle, and wherein a clockwise-facing angle is an angle between a protrusion and a virtual radial spoke extending out from the center of the base. Example variations discussed elsewhere in this disclosure or priority-linked disclosures can be applied to this example where relevant.

FIG. 62 shows an electrode with a base and electroconductive protrusions, wherein protrusions closer to the center connect to the base at a greater clockwise angle than protrusions closer to the center. More specifically, FIG. 62 shows an example of a bottom-up (e.g. distal-to-proximal) view of a dry electrode for use on a hair-covered area comprising: a base 6201; and electroconductive protrusions which extend out from the base; wherein protrusions, including 6202, which are closer to the center of the base connect to the base at a first average clockwise-facing angle, including 6204, wherein protrusions, including 6203, which are farther from the center of the base connect to the base at a second average clockwise-facing angle, including 6205, wherein the second average clockwise-facing angle is less than the first average clockwise-facing angle, and wherein a clockwise-facing angle is an angle between a protrusion and a virtual radial spoke extending out from the center of the base. Example variations discussed elsewhere in this disclosure or priority-linked disclosures can be applied to this example where relevant.

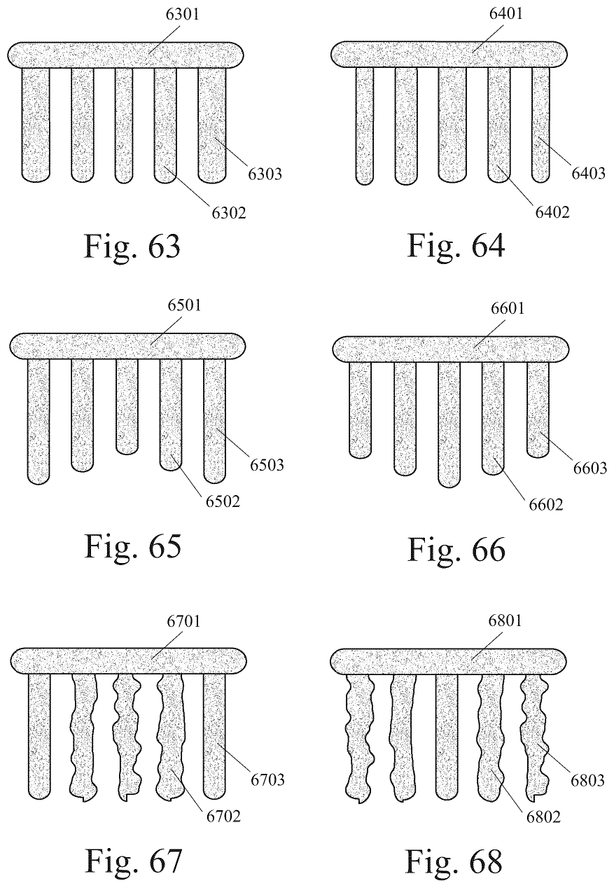

FIG. 63 shows an electrode with a base and electroconductive protrusions, wherein protrusions closer to the center have a smaller cross section. More specifically, FIG. 63 shows an example of a side view of a dry electrode for use on a hair-covered area comprising: a base 6301; and electroconductive protrusions which extend out from the base; wherein protrusions, including 6302, which are closer to the center of the base and/or a midline axis of the base have a first average cross-sectional size, wherein protrusions, including 6303, which are farther from the center of the base and/or a midline axis of the base have a second average cross-sectional size, and wherein the second average cross-sectional size is greater than the first average cross-sectional size. Example variations discussed elsewhere in this disclosure or priority-linked disclosures can be applied to this example where relevant.

FIG. 64 shows an electrode with a base and electroconductive protrusions, wherein protrusions closer to the center have a larger cross section. More specifically, FIG. 64 shows an example of a side view of a dry electrode for use on a hair-covered area comprising: a base 6401; and electroconductive protrusions which extend out from the base; wherein protrusions, including 6402, which are closer to the center of the base and/or a midline axis of the base have a first average cross-sectional size, wherein protrusions, including 6403, which are farther from the center of the base and/or a midline axis of the base have a second average cross-sectional size, and wherein the second average cross-sectional size is less than the first average cross-sectional size. Example variations discussed elsewhere in this disclosure or priority-linked disclosures can be applied to this example where relevant.

FIG. 65 shows an electrode with a base and electroconductive protrusions, wherein protrusions closer to the center are shorter. More specifically, FIG. 65 shows an example of a side view of a dry electrode for use on a hair-covered area comprising: a base 6501; and electroconductive protrusions which extend out from the base; wherein protrusions, including 6502, which are closer to the center of the base and/or a midline axis of the base have a first average length, wherein protrusions, including 6503, which are farther from the center of the base and/or a midline axis of the base have a second average length, and wherein the second average length is greater than the first average length. Example variations discussed elsewhere in this disclosure or priority-linked disclosures can be applied to this example where relevant.

FIG. 66 shows an electrode with a base and electroconductive protrusions, wherein protrusions closer to the center are longer. More specifically, FIG. 66 shows an example of a side view of a dry electrode for use on a hair-covered area comprising: a base 6601; and electroconductive protrusions which extend out from the base; wherein protrusions, including 6602, which are closer to the center of the base and/or a midline axis of the base have a first average length, wherein protrusions, including 6603, which are farther from the center of the base and/or a midline axis of the base have a second average length, and wherein the second average length is less than the first average length. Example variations discussed elsewhere in this disclosure or priority-linked disclosures can be applied to this example where relevant.

FIG. 67 shows an electrode with a base and electroconductive protrusions, wherein protrusions closer to the center are lower durometer. More specifically, FIG. 67 shows an example of a side view of a dry electrode for use on a hair-covered area comprising: a base 6701; and electroconductive protrusions which extend out from the base; wherein protrusions, including 6702, which are closer to the center of the base and/or a midline axis of the base have a first average durometer level; wherein protrusions, including 6703, which are farther from the center of the base and/or a midline axis of the base have a second average durometer level, and wherein the second average durometer level is greater than the first average durometer level. Example variations discussed elsewhere in this disclosure or priority-linked disclosures can be applied to this example where relevant.

FIG. 68 shows an electrode with a base and electroconductive protrusions, wherein protrusions closer to the center are higher durometer. More specifically, FIG. 68 shows an example of a side view of a dry electrode for use on a hair-covered area comprising: a base 6801; and electroconductive protrusions which extend out from the base; wherein protrusions, including 6802, which are closer to the center of the base and/or a midline axis of the base have a first average durometer level; wherein protrusions, including 6803, which are farther from the center of the base and/or a midline axis of the base have a second average durometer level, and wherein the second average durometer level is less than the first average durometer level. Example variations discussed elsewhere in this disclosure or priority-linked disclosures can be applied to this example where relevant.

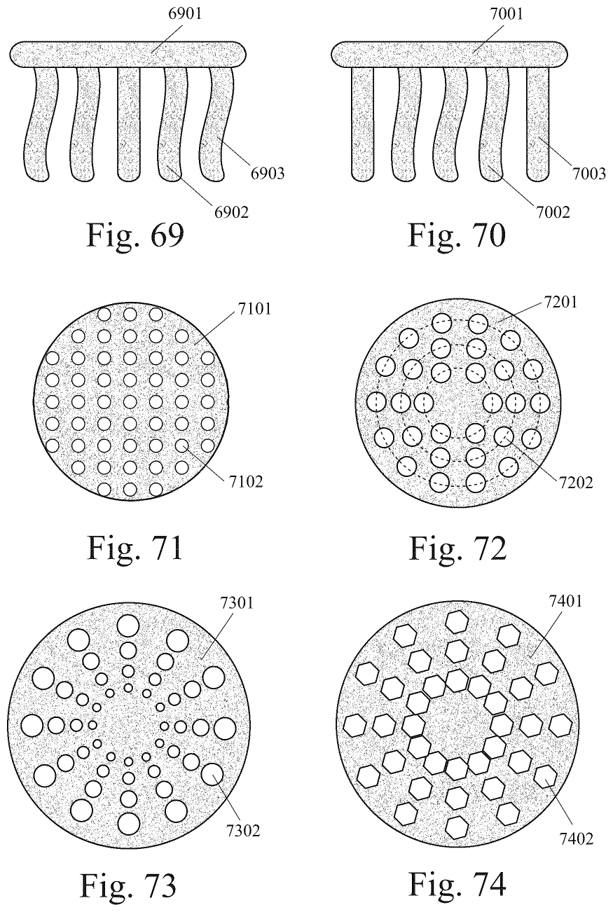

FIG. 69 shows an electrode with a base and electroconductive protrusions, wherein protrusions closer to the center are less elastic or flexible. More specifically, FIG. 69 shows an example of a side view of a dry electrode for use on a hair-covered area comprising: a base 6901; and electroconductive protrusions which extend out from the base; wherein protrusions, including 6902, which are closer to the center of the base and/or a midline axis of the base have a first average elasticity and/or flexibility level; wherein protrusions, including 6903, which are farther from the center of the base and/or a midline axis of the base have a second average elasticity and/or flexibility level, and wherein the second average elasticity and/or flexibility level is greater than the first average elasticity and/or flexibility level. Example variations discussed elsewhere in this disclosure or priority-linked disclosures can be applied to this example where relevant.

FIG. 70 shows an electrode with a base and electroconductive protrusions, wherein protrusions closer to the center are more elastic or flexible. More specifically, FIG. 70 shows an example of a side view of a dry electrode for use on a hair-covered area comprising: a base 7001; and electroconductive protrusions which extend out from the base; wherein protrusions, including 7002, which are closer to the center of the base and/or a midline axis of the base have a first average elasticity and/or flexibility level; wherein protrusions, including 7003, which are farther from the center of the base and/or a midline axis of the base have a second average elasticity and/or flexibility level, and wherein the second average elasticity and/or flexibility level is less than the first average elasticity and/or flexibility level. Example variations discussed elsewhere in this disclosure or priority-linked disclosures can be applied to this example where relevant.

FIG. 71 shows an electrode with a base and electroconductive protrusions, wherein protrusions are evenly-distributed. More specifically, FIG. 71 shows an example of a bottom-up (e.g. distal-to-proximal) view of a dry electrode for use on a hair-covered area comprising: a base 7101; and electroconductive protrusions, including 7102, which extend out from the base, wherein protrusions are evenly-distributed (e.g. evenly-spaced) across the base. Example variations discussed elsewhere in this disclosure or priority-linked disclosures can be applied to this example where relevant.

FIG. 72 shows an electrode with a base and electroconductive protrusions, wherein protrusions are around rings. More specifically, FIG. 72 shows an example of a bottom-up (e.g. distal-to-proximal) view of a dry electrode for use on a hair-covered area comprising: a base 7201; and electroconductive protrusions, including 7202, which extend out from the base, wherein protrusions are distributed along nested (e.g. concentric) rings of protrusions on the base. Example variations discussed elsewhere in this disclosure or priority-linked disclosures can be applied to this example where relevant.

FIG. 73 shows an electrode with a base and electroconductive protrusions, wherein protrusions are along radial spokes. More specifically, FIG. 73 shows an example of a bottom-up (e.g. distal-to-proximal) view of a dry electrode for use on a hair-covered area comprising: a base 7301; and electroconductive protrusions, including 7302, which extend out from the base, wherein protrusions are distributed along virtual radial spokes extending out from the center of the base. Example variations discussed elsewhere in this disclosure or priority-linked disclosures can be applied to this example where relevant.

FIG. 74 shows an electrode with a base and electroconductive protrusions, wherein protrusions are in a hub-and-spoke array. More specifically, FIG. 74 shows an example of a bottom-up (e.g. distal-to-proximal) view of a dry electrode for use on a hair-covered area comprising: a base 7401; and electroconductive protrusions, including 7402, which extend out from the base, wherein protrusions are distributed in a hub-and-spoke array on the base. Example variations discussed elsewhere in this disclosure or priority-linked disclosures can be applied to this example where relevant.

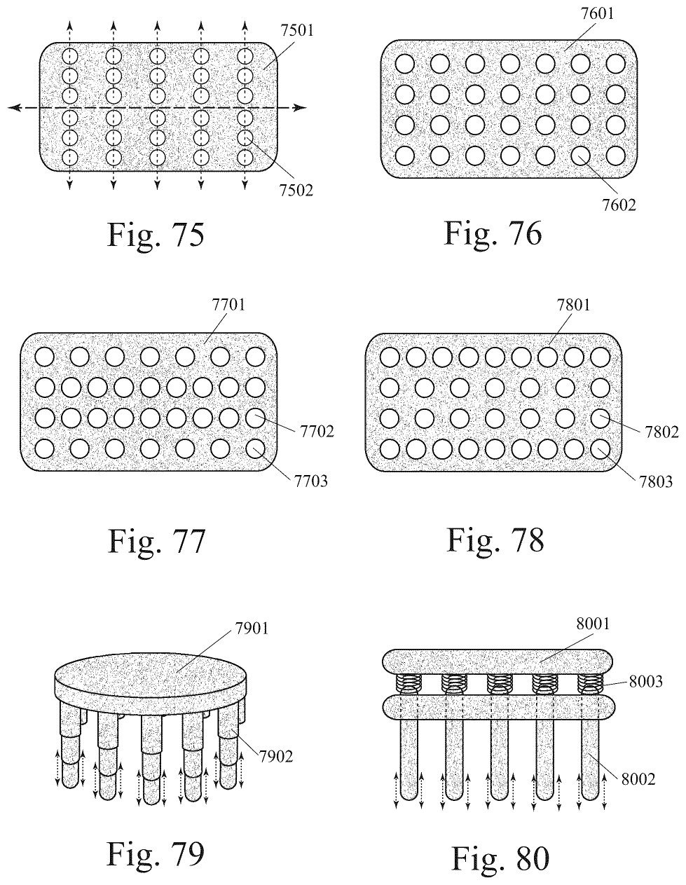

FIG. 75 shows an electrode with a base and electroconductive protrusions, wherein protrusions are along spokes extending out from a midline. More specifically, FIG. 75 shows an example of a bottom-up (e.g. distal-to-proximal) view of a dry electrode for use on a hair-covered area comprising: a base 7501; and electroconductive protrusions, including 7502, which extend out from the base, wherein protrusions are distributed along virtual spokes extending out from a midline of the base. Example variations discussed elsewhere in this disclosure or priority-linked disclosures can be applied to this example where relevant.

FIG. 76 shows an electrode with a base and electroconductive protrusions, wherein protrusions are in a row-and-column array. More specifically, FIG. 76 shows an example of a bottom-up (e.g. distal-to-proximal) view of a dry electrode for use on a hair-covered area comprising: a base 7601; and electroconductive protrusions, including 7602, which extend out from the base, wherein protrusions are distributed in a row-and-column array on the base. Example variations discussed elsewhere in this disclosure or priority-linked disclosures can be applied to this example where relevant.

FIG. 77 shows an electrode with a base and electroconductive protrusions, wherein protrusions closer to the center are closer together. More specifically, FIG. 77 shows an example of a bottom-up (e.g. distal-to-proximal) view of a dry electrode for use on a hair-covered area comprising: a base 7701; and electroconductive protrusions which extend out from the base; wherein protrusions, including 7702, which are closer to the center of the base and/or a midline axis of the base are a first average distance from each other; wherein protrusions, including 7703, which are farther from the center of the base and/or a midline axis of the base are a second average distance from each other, and wherein the second average distance is greater than the first average distance. Example variations discussed elsewhere in this disclosure or priority-linked disclosures can be applied to this example where relevant.

FIG. 78 shows an electrode with a base and electroconductive protrusions, wherein protrusions closer to the center are farther apart. More specifically, FIG. 78 shows an example of a bottom-up (e.g. distal-to-proximal) view of a dry electrode for use on a hair-covered area comprising: a base 7801; and electroconductive protrusions which extend out from the base; wherein protrusions, including 7802, which are closer to the center of the base and/or a midline axis of the base are a first average distance from each other; wherein protrusions, including 7803, which are farther from the center of the base and/or a midline axis of the base are a second average distance from each other, and wherein the second average distance is less than the first average distance. Example variations discussed elsewhere in this disclosure or priority-linked disclosures can be applied to this example where relevant.

FIG. 79 shows an electrode with a base and telescoping electroconductive protrusions. More specifically, FIG. 79 shows an example of an oblique side view of a dry electrode for use on a hair-covered area comprising: a base 7901; and telescoping electroconductive protrusions, including 7902, which extend out from the base. Example variations discussed elsewhere in this disclosure or priority-linked disclosures can be applied to this example where relevant.

FIG. 80 shows an electrode with a base and individually spring-loaded electroconductive protrusions. More specifically, FIG. 80 shows an example of a side view of a dry electrode for use on a hair-covered area comprising: a base 8001; and electroconductive protrusions, including 8002, which extend out from the base, wherein individual protrusions are individually spring loaded (e.g. pushed toward the surface of a person's head by a spring) by individual springs, including 8003. Example variations discussed elsewhere in this disclosure or priority-linked disclosures can be applied to this example where relevant.

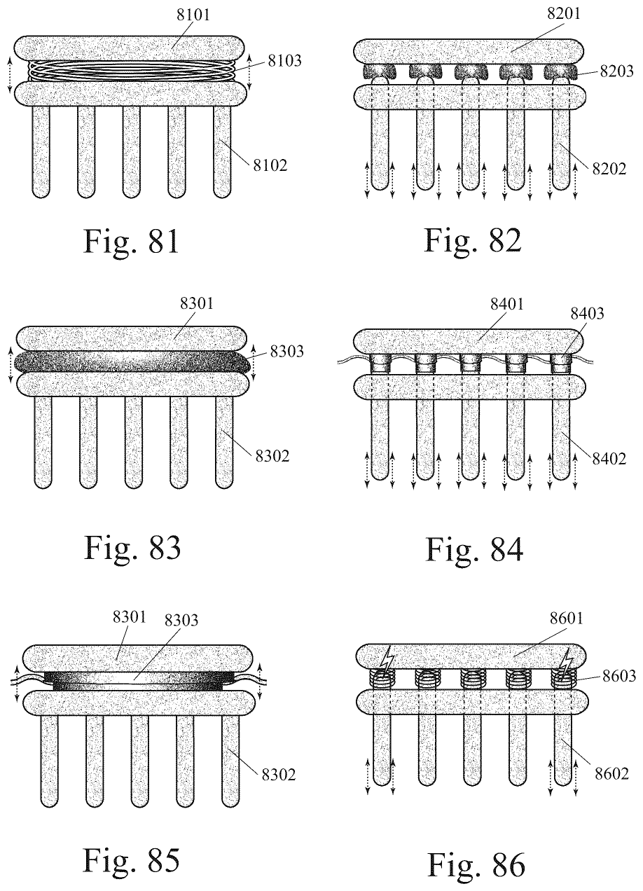

FIG. 81 shows an electrode with a base and collectively spring-loaded electroconductive protrusions. More specifically, FIG. 81 shows an example of a side view of a dry electrode for use on a hair-covered area comprising: a base 8101; and electroconductive protrusions, including 8102, which extend out from the base, wherein protrusions are collectively spring loaded (e.g. pushed toward the surface of a person's head by a spring) by a common spring 8103. Example variations discussed elsewhere in this disclosure or priority-linked disclosures can be applied to this example where relevant.

FIG. 82 shows an electrode with a base and electroconductive protrusions, wherein protrusions are individually pushed toward the surface of a person's head by compressible members. More specifically, FIG. 82 shows an example of a side view of a dry electrode for use on a hair-covered area comprising: a base 8201; and electroconductive protrusions, including 8202, which extend out from the base, wherein individual protrusions are individually pushed toward the surface of a person's head by individual compressible members (e.g. foam pieces or inflatable chambers), including 8203. Example variations discussed elsewhere in this disclosure or priority-linked disclosures can be applied to this example where relevant.

FIG. 83 shows an electrode with a base and electroconductive protrusions, wherein protrusions are collectively pushed toward the surface of a person's head by a compressible member. More specifically, FIG. 83 shows an example of a side view of a dry electrode for use on a hair-covered area comprising: a base 8301; and electroconductive protrusions, including 8302, which extend out from the base, wherein protrusions are collectively pushed toward the surface of a person's head by a common compressible member (e.g. foam piece or inflatable chamber) 8303. Example variations discussed elsewhere in this disclosure or priority-linked disclosures can be applied to this example where relevant.

FIG. 84 shows an electrode with a base and electroconductive protrusions, wherein protrusions are individually pushed toward the surface of a person's head by pneumatic or hydraulic mechanisms More specifically, FIG. 84 shows an example of a side view of a dry electrode for use on a hair-covered area comprising: a base 8401; and electroconductive protrusions, including 8402, which extend out from the base, wherein individual protrusions are individually pushed toward the surface of a person's head by individual pneumatic or hydraulic mechanisms, including 8403. Example variations discussed elsewhere in this disclosure or priority-linked disclosures can be applied to this example where relevant.

FIG. 85 shows an electrode with a base and electroconductive protrusions, wherein protrusions are collectively pushed toward the surface of a person's head by a pneumatic or hydraulic mechanism. More specifically, FIG. 85 shows an example of a side view of a dry electrode for use on a hair-covered area comprising: a base 8501; and electroconductive protrusions, including 8502, which extend out from the base, wherein protrusions are collectively pushed toward the surface of a person's head by a common pneumatic or hydraulic mechanism 8503. Example variations discussed elsewhere in this disclosure or priority-linked disclosures can be applied to this example where relevant.

FIG. 86 shows an electrode with a base and electroconductive protrusions, wherein protrusions are individually pushed toward the surface of a person's head by electromagnetic actuators. More specifically, FIG. 86 shows an example of a side view of a dry electrode for use on a hair-covered area comprising: a base 8601; and electroconductive protrusions, including 8602, which extend out from the base, wherein individual protrusions are individually pushed toward the surface of a person's head by individual electromagnetic actuators (e.g. solenoids), including 8603. Example variations discussed elsewhere in this disclosure or priority-linked disclosures can be applied to this example where relevant.

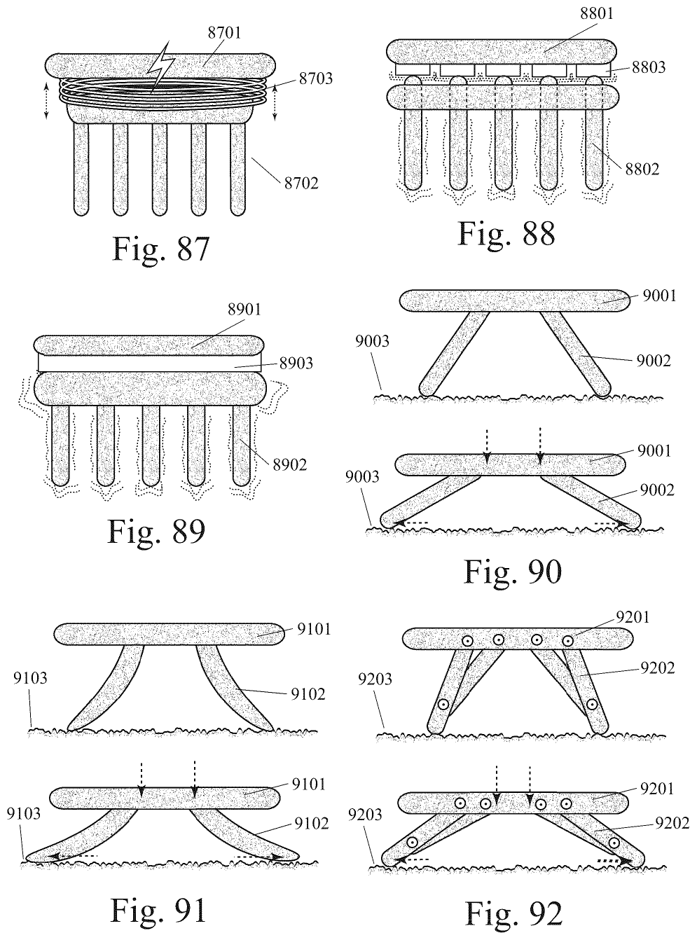

FIG. 87 shows an electrode with a base and electroconductive protrusions, wherein protrusions are collectively pushed toward the surface of a person's head by an electromagnetic actuator. More specifically, FIG. 87 shows an example of a side view of a dry electrode for use on a hair-covered area comprising: a base 8701; and electroconductive protrusions, including 8702, which extend out from the base, wherein protrusions are collectively pushed toward the surface of a person's head by a common electromagnetic actuator (e.g. solenoid) 8703. Example variations discussed elsewhere in this disclosure or priority-linked disclosures can be applied to this example where relevant.

FIG. 88 shows an electrode with a base and electroconductive protrusions, wherein individual protrusions are vibrated and/or oscillated by individual electromagnetic actuators. More specifically, FIG. 88 shows an example of a side view of a dry electrode for use on a hair-covered area comprising: a base 8801; and electroconductive protrusions, including 8802, which extend out from the base, wherein individual protrusions are individually vibrated and/or oscillated along their longitudinal axes by individual electromagnetic actuators (e.g. solenoids), including 8803. Example variations discussed elsewhere in this disclosure or priority-linked disclosures can be applied to this example where relevant.

FIG. 89 shows an electrode with a base and electroconductive protrusions, wherein protrusions are collectively vibrated and/or oscillated by an electromagnetic actuator. More specifically, FIG. 89 shows an example of a side view of a dry electrode for use on a hair-covered area comprising: a base 8901; and electroconductive protrusions, including 8902, which extend out from the base, wherein protrusions are collectively vibrated and/or oscillated along their longitudinal axes by a common electromagnetic actuator (e.g. solenoid) 8903. Example variations discussed elsewhere in this disclosure or priority-linked disclosures can be applied to this example where relevant.

FIG. 90 shows an electrode with a base and electroconductive protrusions, wherein pushing protrusions toward the surface of a person's head spreads their tips farther apart. More specifically, the upper and lower portions of FIG. 90 show an example of two sequential side views of a dry electrode for use on a hair-covered area comprising: a base 9001; and electroconductive protrusions, including 9002, which extend out from the base, wherein the protrusions are configured so that pushing them toward the surface of the person's head 9003 causes their distal tips to move farther apart from each other (in a direction parallel to the surface of the person's head). Example variations discussed elsewhere in this disclosure or priority-linked disclosures can be applied to this example where relevant.

FIG. 91 shows an electrode with a base and electroconductive protrusions, wherein protrusions are shaped so that pushing them toward the surface of a person's head spreads their tips farther apart. More specifically, the upper and lower portions of FIG. 91 show an example of two sequential side views of a dry electrode for use on a hair-covered area comprising: a base 9101; and electroconductive protrusions, including 9102, which extend out from the base, wherein the protrusions are shaped so that pushing them toward the surface of the person's head 9103 causes their distal tips to move farther apart from each other (in a direction parallel to the surface of the person's head). Example variations discussed elsewhere in this disclosure or priority-linked disclosures can be applied to this example where relevant.

FIG. 92 shows an electrode with a base and articulated electroconductive protrusions, wherein pushing protrusions toward the surface of a person's head spreads their tips farther apart. More specifically, the upper and lower portions of FIG. 92 show an example of two sequential side views of a dry electrode for use on a hair-covered area comprising: a base 9201; and electroconductive protrusions, including 9202, which extend out from the base, wherein the protrusions are articulated so that pushing them toward the surface of the person's head 9203 causes their distal tips to move farther apart from each other (in a direction parallel to the surface of the person's head). Example variations discussed elsewhere in this disclosure or priority-linked disclosures can be applied to this example where relevant.

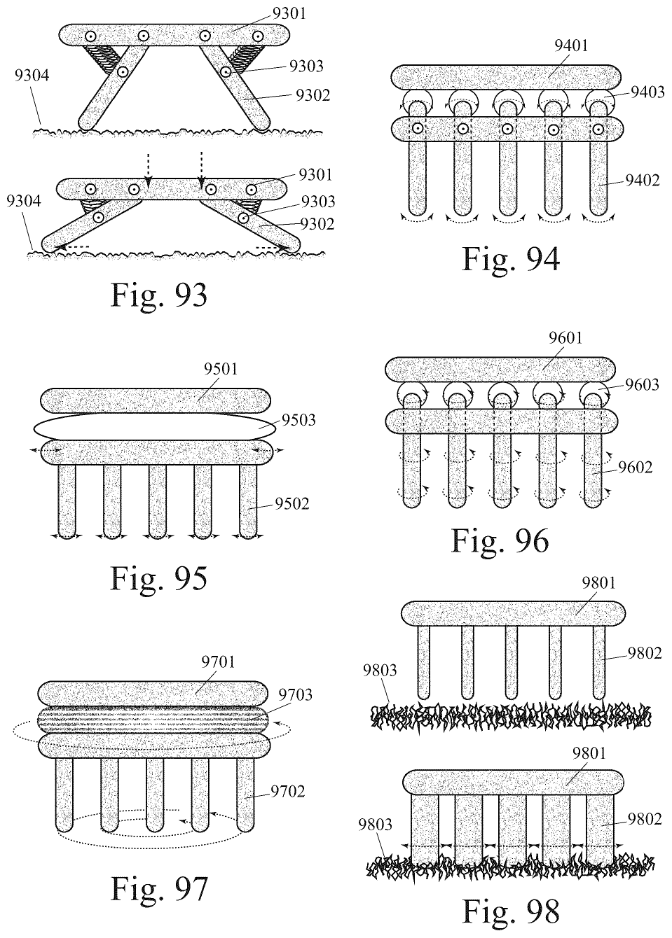

FIG. 93 shows an electrode with a base and electroconductive protrusions with hinges or joints, wherein pushing protrusions toward the surface of a person's head spreads their tips farther apart. More specifically, the upper and lower portions of FIG. 93 show an example of two sequential side views of a dry electrode for use on a hair-covered area comprising: a base 9301; and electroconductive protrusions, including 9302, which extend out from the base, wherein the protrusions have hinges or movable joints, including 9303, so that pushing them toward the surface of the person's head 9304, causes their distal tips to move farther apart from each other (in a direction parallel to the surface of the person's head). Example variations discussed elsewhere in this disclosure or priority-linked disclosures can be applied to this example where relevant.

FIG. 94 shows an electrode with a base and electroconductive protrusions, wherein protrusions are individually vibrated and/or oscillated laterally by electromagnetic actuators. More specifically, FIG. 94 shows an example of a side view of a dry electrode for use on a hair-covered area comprising: a base 9401; and electroconductive protrusions, including 9402, which extend out from the base, wherein individual protrusions are individually vibrated and/or oscillated laterally (across the surface of a person's head) by individual electromagnetic actuators (e.g. solenoids), including 9403. Example variations discussed elsewhere in this disclosure or priority-linked disclosures can be applied to this example where relevant.

FIG. 95 shows an electrode with a base and electroconductive protrusions, wherein protrusions are collectively vibrated and/or oscillated laterally by an electromagnetic actuator. More specifically, FIG. 95 shows an example of a side view of a dry electrode for use on a hair-covered area comprising: a base 9501; and electroconductive protrusions, including 9502, which extend out from the base, wherein protrusions are collectively vibrated and/or oscillated laterally (across the surface of a person's head) by a common electromagnetic actuator (e.g. solenoid) 9503. Example variations discussed elsewhere in this disclosure or priority-linked disclosures can be applied to this example where relevant.

FIG. 96 shows an electrode with a base and electroconductive protrusions, wherein protrusions are individually rotated. More specifically, FIG. 96 shows an example of a side view of a dry electrode for use on a hair-covered area comprising: a base 9601; and electroconductive protrusions, including 9602, which extend out from the base, wherein individual protrusions are individually rotated (e.g. complete rotation or rotational oscillation) around their longitudinal axes by individual electromagnetic actuators, including 9603. Example variations discussed elsewhere in this disclosure or priority-linked disclosures can be applied to this example where relevant.

FIG. 97 shows an electrode with a base and electroconductive protrusions, wherein protrusions are collectively rotated. More specifically, FIG. 97 shows an example of a side view of a dry electrode for use on a hair-covered area comprising: a base 9701; and electroconductive protrusions, including 9702, which extend out from the base, wherein protrusions are collectively rotated (e.g. complete rotation or rotational oscillation) around a central axis of the base by an electromagnetic actuator 9703. Example variations discussed elsewhere in this disclosure or priority-linked disclosures can be applied to this example where relevant.

FIG. 98 shows an electrode with a base and electroconductive protrusions, wherein the cross-sectional size of protrusions is increased after their insertion between strands of hair. More specifically, the upper and lower portions of FIG. 98 show an example of two sequential side views of a dry electrode for use on a hair-covered area comprising: a base 9801; and electroconductive protrusions, including 9802, which extend out from the base, wherein protrusions have a first configuration with a first average cross-sectional size, wherein protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head 9803. Example variations discussed elsewhere in this disclosure or priority-linked disclosures can be applied to this example where relevant.

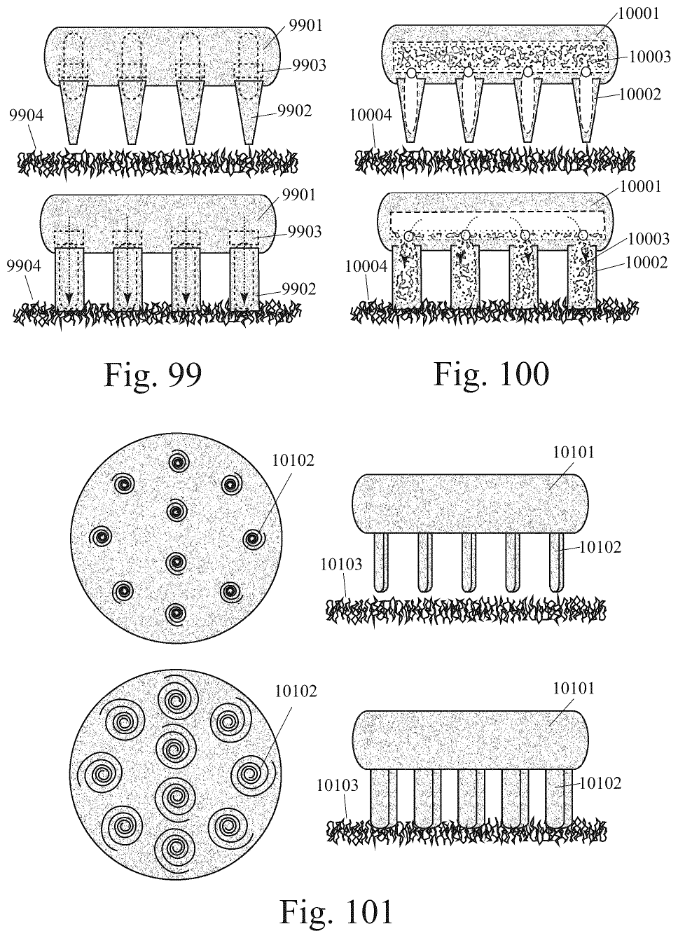

FIG. 99 shows an electrode with a base and electroconductive protrusions, wherein the cross-sectional size of protrusions is increased by electromagnetic actuators after their insertion between strands of hair. More specifically, the upper and lower portions of FIG. 99 show an example of two sequential side views of a dry electrode for use on a hair-covered area comprising: a base 9901; and electroconductive protrusions, including 9902, which extend out from the base, wherein protrusions have a first configuration with a first average cross-sectional size, wherein protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head 9904 by one or more electromagnetic actuators, including 9903. Example variations discussed elsewhere in this disclosure or priority-linked disclosures can be applied to this example where relevant.

FIG. 100 shows an electrode with a base and electroconductive protrusions, wherein the cross-sectional size of protrusions is increased by inserting material into their interiors after insertion of the protrusions between strands of hair. More specifically, the upper and lower portions of FIG. 100 show an example of two sequential side views of a dry electrode for use on a hair-covered area comprising: a base 10001; and electroconductive protrusions, including 10002, which extend out from the base, wherein protrusions have a first configuration with a first average cross-sectional size, wherein protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head 10004 by insertion of material 10003 into the interiors of the protrusions. Example variations discussed elsewhere in this disclosure or priority-linked disclosures can be applied to this example where relevant.

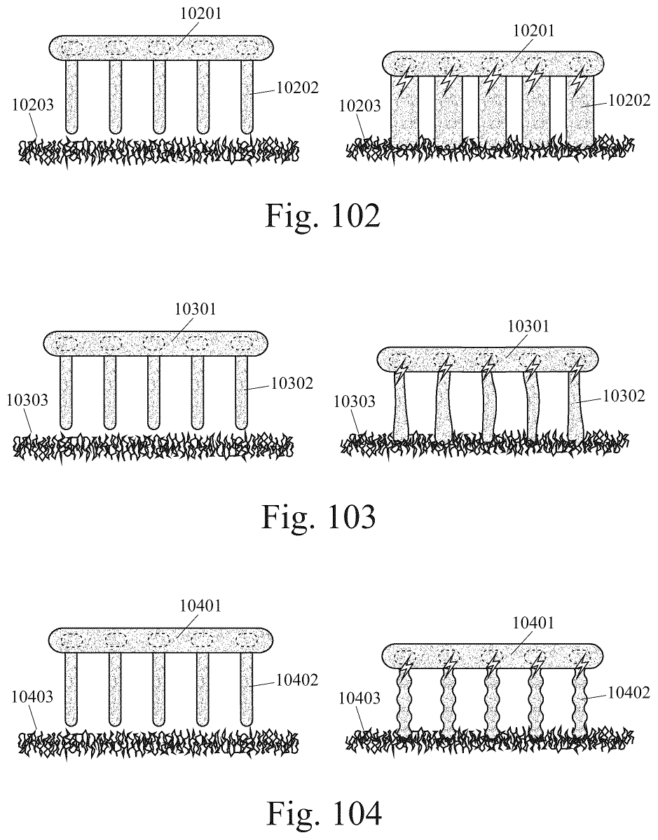

FIG. 101 shows an electrode with a base and electroconductive protrusions, wherein the cross-sectional size of protrusions is increased by uncoiling them after inserting them between strands of hair. More specifically, the upper and lower left-side portions of FIG. 101 show an example of two sequential bottom-up views and the upper and lower right-side portions of FIG. 101 show an example of two sequential side views of a dry electrode for use on a hair-covered area comprising: a base 10101; and electroconductive protrusions, including 10102, which extend out from the base, wherein protrusions have a first configuration with a first average cross-sectional size, wherein protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head 10103 by uncoiling the protrusions. Example variations discussed elsewhere in this disclosure or priority-linked disclosures can be applied to this example where relevant.

FIG. 102 shows an electrode with a base and electroconductive protrusions, wherein the cross-sectional size of protrusions is increased by application of electrical energy after inserting the protrusions between strands of hair. More specifically, the left and right portions of FIG. 102 show an example of two sequential side views of a dry electrode for use on a hair-covered area comprising: a base 10201; and electroconductive protrusions, including 10202, which extend out from the base, wherein protrusions have a first configuration with a first average cross-sectional size, wherein protrusions have a second configuration with a second cross-sectional size, wherein the second cross-sectional size is greater than the first cross-sectional size, and wherein the protrusions are changed from the first configuration to the second configuration after the protrusions have been inserted between strands of hair on the surface of a person's head 10203 by the application of electrical energy. Example variations discussed elsewhere in this disclosure or priority-linked disclosures can be applied to this example where relevant.

FIG. 103 shows an electrode with a base and electroconductive protrusions, wherein the durometer of protrusions is decreased after inserting them between strands of hair. More specifically, the left and right portions of FIG. 103 show an example of two sequential side views of a dry electrode for use on a hair-covered area comprising: a base 10301; and electroconductive protrusions, including 10302, which extend out from the base, wherein protrusions have a first configuration with a first durometer level, wherein protrusions have a second configuration with a second durometer level, wherein the second durometer level is less than the first durometer level, and wherein the protrusions are changed from the first configuration to the second configuration after the protrusions have been inserted between strands of hair on the surface of a person's head 10303 by the application of electrical energy. Example variations discussed elsewhere in this disclosure or priority-linked disclosures can be applied to this example where relevant.

FIG. 104 shows an electrode with a base and electroconductive protrusions, wherein the flexibility or elasticity of protrusions is increased after inserting them between strands of hair. More specifically, the left and right portions of FIG. 104 show an example of two sequential side views of a dry electrode for use on a hair-covered area comprising: a base 10401; and electroconductive protrusions, including 10402, which extend out from the base, wherein protrusions have a first configuration with a first flexibility or elasticity level, wherein protrusions have a second configuration with a second flexibility or elasticity level, wherein the second flexibility or elasticity level is greater than the first flexibility or elasticity level, and wherein the protrusions are changed from the first configuration to the second configuration after the protrusions have been inserted between strands of hair on the surface of a person's head 10403 by the application of electrical energy. Example variations discussed elsewhere in this disclosure or priority-linked disclosures can be applied to this example where relevant.

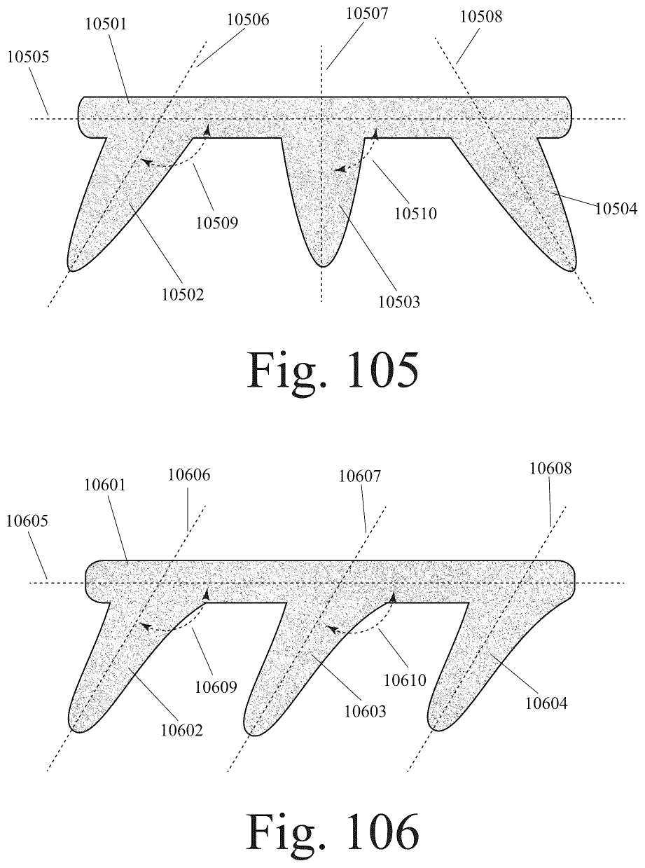

FIG. 105 shows a side cross-sectional view of an EEG electrode for use on a hair-covered portion of a person's head comprising: (a) a (disk-shaped) distal portion 10501 with a central cross-sectional plane 10505 (which is substantially parallel to the surface of a person's head); and (b) a plurality of electroconductive proximal protrusions (e.g. pins, prongs, teeth, spikes, fingers, and/or protrusions), including protrusions 10502, 10503, and 10504, which extend out from the distal portion toward the person's head in order to penetrate between hairs and come into contact with the surface of the person's head; wherein each protrusion in the plurality of electroconductive proximal protrusions has a central longitudinal axis, including axes 10506, 10507, and 10508; wherein a linear extension of a first central longitudinal axis 10507 of a first protrusion 10503 in the plurality of conductive proximal protrusions intersects the central cross-sectional plane of the distal portion at a first distance (which can be zero) from a center of the distal portion; wherein a linear extension of a second central longitudinal axis 10506 of a second protrusion 10502 in the plurality of conductive proximal protrusions intersects the central cross-sectional plane of the distal portion at a second distance from the center of the distal portion; wherein the second distance is greater than the first distance; wherein a protrusion angle is the angle of intersection between a linear extension of a central longitudinal axis of a protrusion and the cross-sectional plane of the distal portion which faces toward, or aligns with, the center of the cross-sectional plane of the distal portion; wherein a second protrusion angle 10509 of the second protrusion 10502 is greater than a first protrusion angle 10510 of the first protrusion 10503.

In an example, a distal portion of an electrode can have a circular and/or disk shape. In an example, the distal portion can have an oblong, oval, or elliptical shape. In an example, the distal portion can have a square or rectangular shape. In an example, the distal portion can have a rounded square or rounded rectangular shape. In an example, the distal portion can have a hexagonal shape. In an example, the distal portion of the electrode can have non-uniform thickness. In an example, the center of the distal portion can be thicker than the periphery of the distal portion. In an example, the center of the distal portion can be thinner than the periphery of the distal portion. In an example, the distal portion of the electrode can be electroconductive. In an example, a distal portion of an electrode can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal.

In an example, a plurality of electroconductive proximal protrusions (e.g. pins, prongs, teeth, spikes, fingers, and/or protrusions) can include a protrusion from the center of a distal portion of an electrode. In an example, the protrusion angle of a protrusion at the center of the distal portion can be 90 degrees. In an example, the protrusion angle of non-central protrusion can be in the range of 100 to 145 degrees. In an example, the protrusion angle of non-central protrusion can be in the range of 130 to 160 degrees. In an example, the protrusion angle of non-central protrusion can be at least 10 degrees greater than the protrusion angle of a protrusion at the center of the distal portion. In an example, the protrusion angle of non-central protrusion can be at least 30 degrees greater than the protrusion angle of a protrusion at the center of the distal portion. In an example, protrusions which are farther from the center of the electrode can tilt, point, bow, or curve away from the center of the electrode.

In an example, protrusions which are closer to the center of a distal portion of an electrode can be longer than protrusions which are farther from the center of the distal portion. In an example, protrusions which are farther from the center of a distal portion of an electrode can be longer than protrusions which are closer to the center of the distal portion. In an example, protrusions which are closer to the center of a distal portion of an electrode can be more flexible, be more elastic, have a higher Young's modulus, be more compressible, and/or have a lower durometer than protrusions which are farther from the center of the distal portion. In an example, protrusions which are farther from the center of a distal portion of an electrode can be more flexible, be more elastic, have a higher Young's modulus, be more compressible, and/or have a lower durometer than protrusions which are closer to the center of the distal portion.

In an example, a plurality of electroconductive proximal protrusions (e.g. pins, prongs, teeth, spikes, fingers, and/or protrusions) can comprise at least two nested (e.g. concentric) rings of protrusions. In an example, the protrusion angles of protrusions in an outer ring can be greater than the protrusion angles of protrusions in an inner ring. In an example, the protrusion angles of protrusions in an outer ring can be less than the protrusion angles of protrusions in an inner ring. In an example, there can be differences and/or variation in protrusion angles and/or protrusion directions of different protrusions around the circumference of a ring of protrusions. In an example, a plurality of electroconductive proximal protrusions (e.g. pins, prongs, teeth, spikes, fingers, and/or protrusions) can comprise a hub-and-spoke array of protrusions.

In an example, an EEG electrode can comprise a distal portion, a conductive protrusion which extends out from the center of the distal portion toward a person's head, and a ring of conductive protrusions around the central protrusion which also extend out from the distal portion toward the person's head. In an example, an EEG electrode can comprise a distal portion, a conductive protrusion which extends out from the center of the distal portion toward a person's head, and a ring of conductive protrusions around the periphery of the distal portion which also extend out from the distal portion toward the person's head. In an example, an EEG electrode can comprise a distal portion, a conductive protrusion which extends out from the center of the distal portion toward a person's head, and two rings of conductive protrusions around the central protrusion which also extend out from the distal portion toward the person's head. In an example, an EEG electrode can comprise a distal portion and two or more nested rings of conductive protrusions which extend out from the distal portion toward the person's head, wherein protrusions in an outer ring protrude from the distal portion at a greater center-facing angle than protrusions in an inner ring.

In an example, a plurality of electroconductive proximal protrusions (e.g. pins, prongs, teeth, spikes, fingers, and/or protrusions) can comprise an orthogonal grid (e.g. with at least two sets of orthogonal rows and columns) of protrusions. In an example, there can be differences and/or variation in protrusion angles and/or protrusion directions of different protrusions along a row or column of protrusions. In an example, a plurality of electroconductive proximal protrusions (e.g. pins, prongs, teeth, spikes, fingers, and/or protrusions) can comprise a hexagonal grid (e.g. a honeycomb-shaped array) of protrusions.

In an example, a protrusion can have a columnar shape. In an example, a protrusion can have a conic shape. In an example, a protrusion can have a conic section shape. In an example, a protrusion can have a frustal shape. In an example, a protrusion can have a parabolic shape. In an example, a protrusion can have an ellipsoidal shape. In an example, a protrusion can have a crescent and/or banana shape. In an example, a protrusion can have a pyramidic shape. In an example, a protrusion can have a hemispherical shape. In an example, a protrusion can have a distal (farther from head) hemispherical portion and a proximal (closer to head) frustum-shaped portion. In an example, a protrusion can have a distal hemispherical portion and a proximal paraboloid-shaped portion.

In an example, a hair-penetrating protrusion on an electrode can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal. Example variations discussed elsewhere in this disclosure or priority-linked disclosures can be applied to this example where relevant.

FIG. 106 shows a side cross-sectional view of an EEG electrode for use on a hair-covered portion of a person's head comprising: (a) a (disk-shaped) distal portion 10601 with a central cross-sectional plane 10605 (which is substantially parallel to the surface of a person's head); and (b) a plurality of electroconductive proximal protrusions (e.g. pins, prongs, teeth, spikes, fingers, and/or protrusions), including protrusions 10602, 10603, and 10604, which extend out from the distal portion toward the person's head in order to penetrate between hairs and come into contact with the surface of the person's head; wherein each protrusion in the plurality of electroconductive proximal protrusions has a central longitudinal axis, including axes 10606, 10607, and 10608; wherein a linear extension of a first central longitudinal axis 10607 of a first protrusion 10603 in the plurality of conductive proximal protrusions intersects the central cross-sectional plane of the distal portion at a first distance (which can be zero) from a center of the distal portion; wherein a linear extension of a second central longitudinal axis 10606 of a second protrusion 10602 in the plurality of conductive proximal protrusions intersects the central cross-sectional plane of the distal portion at a second distance from the center of the distal portion; wherein the second distance is greater than the first distance; wherein a protrusion angle is the angle of intersection between a linear extension of a central longitudinal axis of a protrusion and the cross-sectional plane of the distal portion which faces toward, or aligns with, the center of the cross-sectional plane of the distal portion; wherein a second protrusion angle 10609 of the second protrusion 10602 is equal to a first protrusion angle 10610 of the first protrusion 10603; and wherein the first protrusion angle is within the range of 95 to 160 degrees.

In an example, a distal portion of an electrode can have a circular and/or disk shape. In an example, the distal portion can have an oblong, oval, or elliptical shape. In an example, the distal portion can have a square or rectangular shape. In an example, the distal portion can have a rounded square or rounded rectangular shape. In an example, the distal portion can have a hexagonal shape. In an example, the distal portion of the electrode can have non-uniform thickness. In an example, the center of the distal portion can be thicker than the periphery of the distal portion. In an example, the center of the distal portion can be thinner than the periphery of the distal portion. In an example, the distal portion of the electrode can be electroconductive. In an example, a distal portion of an electrode can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal.

In an example, a plurality of electroconductive proximal protrusions (e.g. pins, prongs, teeth, spikes, fingers, and/or protrusions) can include a protrusion from the center of a distal portion of an electrode. In an example, protrusions which are closer to the center of a distal portion of an electrode can be longer than protrusions which are farther from the center of the distal portion. In an example, protrusions which are farther from the center of a distal portion of an electrode can be longer than protrusions which are closer to the center of the distal portion. In an example, protrusions which are closer to the center of a distal portion of an electrode can be more flexible, be more elastic, have a higher Young's modulus, be more compressible, and/or have a lower durometer than protrusions which are farther from the center of the distal portion. In an example, protrusions which are farther from the center of a distal portion of an electrode can be more flexible, be more elastic, have a higher Young's modulus, be more compressible, and/or have a lower durometer than protrusions which are closer to the center of the distal portion.

In an example, a plurality of electroconductive proximal protrusions (e.g. pins, prongs, teeth, spikes, fingers, and/or protrusions) can comprise at least two nested (e.g. concentric) rings of protrusions. In an example, a plurality of electroconductive proximal protrusions (e.g. pins, prongs, teeth, spikes, fingers, and/or protrusions) can comprise a hub-and-spoke array of protrusions. In an example, a plurality of electroconductive proximal protrusions (e.g. pins, prongs, teeth, spikes, fingers, and/or protrusions) can comprise an orthogonal grid (e.g. with at least two sets of orthogonal rows and columns) of protrusions. In an example, a plurality of electroconductive proximal protrusions (e.g. pins, prongs, teeth, spikes, fingers, and/or protrusions) can comprise a hexagonal grid (e.g. honeycomb-shaped array) of protrusions.

In an example, an electrode can comprise: a circular distal portion; and an array of conductive protrusions which extend out from the distal portion toward the surface of a person's head, wherein all of the protrusions are angled, tilted, and/or bent toward the same half of the circumference of distal portion. In an example, an electrode can comprise: a circular distal portion; and an array of conductive protrusions which extend out from the distal portion toward the surface of a person's head, wherein all of the protrusions are angled, tilted, and/or bent toward the same quadrant of the circumference of distal portion. In an example, an electrode can comprise: a quadrilateral distal portion; and an array of conductive protrusions which extend out from the distal portion toward the surface of a person's head, wherein all of the protrusions are angled, tilted, and/or bent toward the same side of the quadrilateral distal portion.

In an example, a protrusion can have a columnar shape. In an example, a protrusion can have a conic shape. In an example, a protrusion can have a conic section shape. In an example, a protrusion can have a frustal shape. In an example, a protrusion can have a parabolic shape. In an example, a protrusion can have an ellipsoidal shape. In an example, a protrusion can have a crescent and/or banana shape. In an example, a protrusion can have a pyramidic shape. In an example, a protrusion can have a hemispherical shape. In an example, a protrusion can have a distal (farther from head) hemispherical portion and a proximal (closer to head) frustum-shaped portion. In an example, a protrusion can have a distal hemispherical portion and a proximal paraboloid-shaped portion.

In an example, a hair-penetrating protrusion on an electrode can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal. Example variations discussed elsewhere in this disclosure or priority-linked disclosures can be applied to this example where relevant.

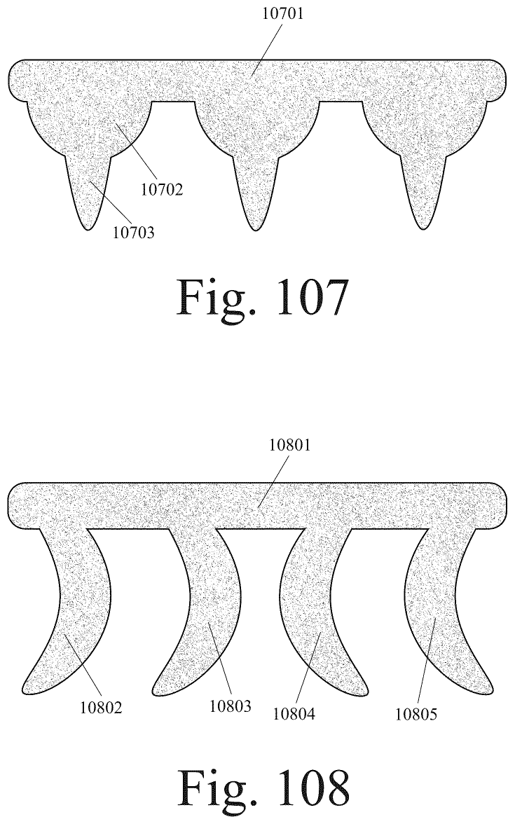

FIG. 107 shows a side cross-sectional view of an EEG electrode for use on a hair-covered portion of a person's head comprising: a (disk-shaped) distal portion 10701; and a plurality of electroconductive proximal protrusions (e.g. pins, prongs, teeth, spikes, fingers, and/or protrusions) which extend out from the distal portion toward the person's head in order to penetrate between hairs and come into contact with the surface of the person's head; wherein a protrusion in the plurality of electroconductive proximal protrusions further comprises a hemispherical distal portion 10702 (which is farther from the surface of the person's head) and a paraboloid-shaped proximal portion 10703 (which is closer to the surface of the person's head). In an example, a protrusion can have a shape like an ice cream cone with a scoop of ice cream on it. In an example, a protrusion can have a shape like a champagne glass without a base on the stem.

In an example, a distal portion of an electrode can have a circular and/or disk shape. In an example, the distal portion can have an oblong, oval, or elliptical shape. In an example, the distal portion can have a square or rectangular shape. In an example, the distal portion can have a rounded square or rounded rectangular shape. In an example, the distal portion can have a hexagonal shape. In an example, the distal portion of the electrode can have non-uniform thickness. In an example, the center of the distal portion can be thicker than the periphery of the distal portion. In an example, the center of the distal portion can be thinner than the periphery of the distal portion. In an example, the distal portion of the electrode can be electroconductive. In an example, a distal portion of an electrode can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal.

In an example, a plurality of electroconductive proximal protrusions (e.g. pins, prongs, teeth, spikes, fingers, and/or protrusions) can comprise at least two nested (e.g. concentric) rings of protrusions. In an example, a plurality of electroconductive proximal protrusions (e.g. pins, prongs, teeth, spikes, fingers, and/or protrusions) can comprise a hub-and-spoke array of protrusions. In an example, a plurality of electroconductive proximal protrusions (e.g. pins, prongs, teeth, spikes, fingers, and/or protrusions) can comprise an orthogonal grid (e.g. with at least two sets of orthogonal rows and columns) of protrusions. In an example, a plurality of electroconductive proximal protrusions (e.g. pins, prongs, teeth, spikes, fingers, and/or protrusions) can comprise a hexagonal grid (e.g. honeycomb-shaped array) of protrusions.

In an example, a hair-penetrating protrusion on an electrode can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal. Example variations discussed elsewhere in this disclosure or priority-linked disclosures can be applied to this example where relevant.

FIG. 108 shows a side cross-sectional view of an EEG electrode for use on a hair-covered portion of a person's head comprising: a (disk-shaped) distal portion 10801; and a plurality of crescent and/or banana-shaped electroconductive proximal protrusions (e.g. pins, prongs, teeth, spikes, fingers, and/or protrusions), including protrusions 10802, 10803, 10804, and 10805, which extend out from the distal portion toward the person's head in order to penetrate between hairs and come into contact with the surface of the person's head; wherein protrusions in the plurality of crescent and/or banana-shaped electroconductive proximal protrusions have curved sides which bow inward toward the center of the distal portion.

In an example, a distal portion of an electrode can have a circular and/or disk shape. In an example, a distal portion of an electrode can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal.

In an example, a plurality of electroconductive proximal protrusions (e.g. pins, prongs, teeth, spikes, fingers, and/or protrusions) can comprise at least two nested (e.g. concentric) rings of protrusions. In an example, a plurality of electroconductive proximal protrusions (e.g. pins, prongs, teeth, spikes, fingers, and/or protrusions) can comprise a hub-and-spoke array of protrusions.

In an example, a protrusion can have a concave side which faces away from the center of the distal portion and a convex side which faces toward the center of the distal portion. In an example, a protrusion can have a shape like a hanging banana. In an example, protrusions on one side of a cross-section of an electrode can curve in a first direction and protrusions on the other side of the cross-section of the electrode can curve in the opposite direction. In an example, the proximal ends of crescent or banana-shaped protrusions can all point away from the central proximal-to-distal axis of the electrode. In an example, the proximal ends of arcuate protrusions can all point away from the central proximal-to-distal axis of the electrode. In an example, protrusions which are farther from the center of the electrode can tilt, point, bow, or curve away from the center of the electrode.

In an example, a hair-penetrating protrusion on an electrode can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal. Example variations discussed elsewhere in this disclosure or priority-linked disclosures can be applied to this example where relevant.

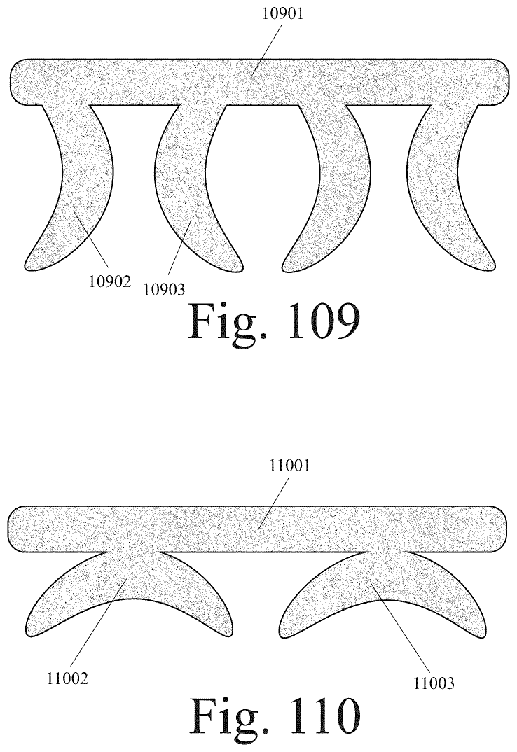

FIG. 109 shows a side cross-sectional view of an EEG electrode for use on a hair-covered portion of a person's head comprising: a (disk-shaped) distal portion 10901; and at least two nested rings of crescent and/or banana-shaped electroconductive proximal protrusions (e.g. pins, prongs, teeth, spikes, fingers, and/or protrusions) which extend out from the distal portion toward the person's head in order to penetrate between hairs and come into contact with the surface of the person's head; wherein protrusions in a first (e.g. inner) ring, including protrusion 10903, have curved sides which bow outward away from the center of the distal portion; and wherein protrusions in a second (outer) ring, including protrusion 10902, have curved sides which bow inward toward the center of the distal portion.

In an example, a distal portion of an electrode can have a circular and/or disk shape. In an example, a hair-penetrating protrusion can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal. Example variations discussed elsewhere in this disclosure or priority-linked disclosures can be applied to this example where relevant.

FIG. 110 shows a side cross-sectional view of an EEG electrode for use on a hair-covered portion of a person's head comprising: a (disk-shaped) distal portion 11001; and a plurality of crescent and/or banana-shaped electroconductive proximal protrusions (e.g. pins, prongs, teeth, spikes, fingers, and/or protrusions) which extend out from the distal portion toward the person's head in order to penetrate between hairs and come into contact with the surface of the person's head; wherein the protrusions, including protrusions 11002 and 11003, have curved sides which bow (upward) away from the surface of the person's head. In an example, the longitudinal axes of crescent or banana shaped protrusions can be substantially parallel to the plane of the distal portion of the electrode.

In an example, a distal portion of an electrode can have a circular and/or disk shape. In an example, a hair-penetrating protrusion can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal. Example variations discussed elsewhere in this disclosure or priority-linked disclosures can be applied to this example where relevant.

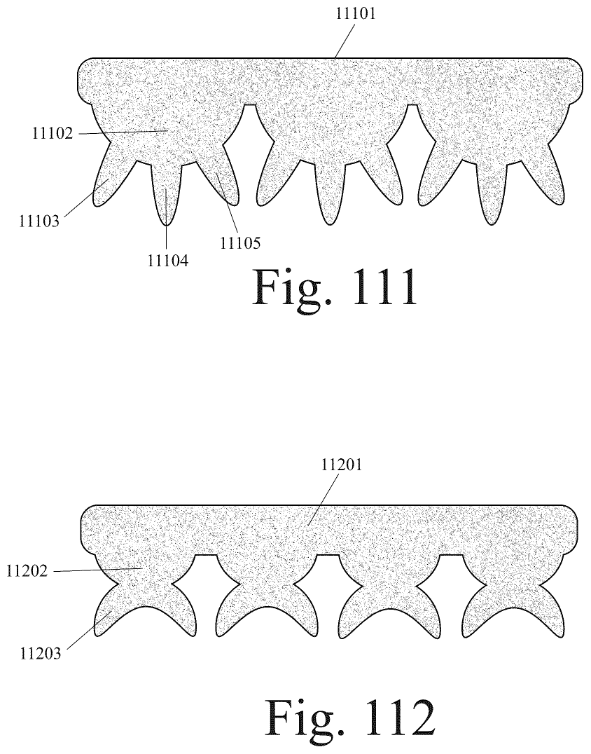

FIG. 111 shows a side cross-sectional view of an EEG electrode for use on a hair-covered portion of a person's head comprising: a (disk-shaped) distal portion 11101; and a plurality of electroconductive proximal protrusions (e.g. pins, prongs, teeth, spikes, fingers, and/or protrusions) which extend out from the distal portion toward the person's head in order to penetrate between hairs and come into contact with the surface of the person's head; wherein a protrusion in the plurality of electroconductive proximal protrusions further comprises a hemispherical distal (farther from the person's head) portion 11102 and a plurality of paraboloidal proximal (closer to the person's head) portions 11103, 11104, and 11105. In other words, this example is an EEG electrode with a bovinely inspired topography which is udderly conductive.

In an example, a distal portion of an electrode can have a circular and/or disk shape. In an example, a hair-penetrating protrusion can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal. Example variations discussed elsewhere in this disclosure or priority-linked disclosures can be applied to this example where relevant.

FIG. 112 shows a side cross-sectional view of an EEG electrode for use on a hair-covered portion of a person's head comprising: a (disk-shaped) distal portion 11201; and a plurality of electroconductive proximal protrusions (e.g. pins, prongs, teeth, spikes, fingers, and/or protrusions) which extend out from the distal portion toward the person's head in order to penetrate between hairs and come into contact with the surface of the person's head; wherein a protrusion in the plurality of electroconductive proximal protrusions further comprises a hemispherical distal (farther from the person's head) portion 11202 and a crescent or banana-shaped proximal (closer to the person's head) portion 11203.

In an example, a distal portion of an electrode can have a circular and/or disk shape. In an example, a hair-penetrating protrusion can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal. Example variations discussed elsewhere in this disclosure or priority-linked disclosures can be applied to this example where relevant.

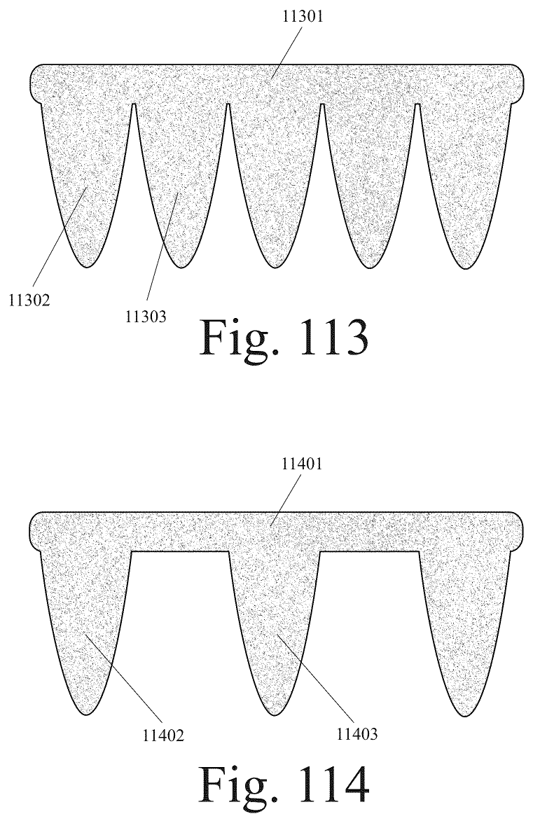

FIG. 113 shows a side cross-sectional view of an EEG electrode for use on a hair-covered portion of a person's head comprising: a (disk-shaped) distal portion 11301; and a plurality of paraboloid-shaped proximal protrusions, including 11302 and 11303, which extend out from the distal portion toward the person's head in order to penetrate between hairs and come into contact with the surface of the person's head. In this example, there are six protrusions in a side cross-section of the electrode. Example variations discussed elsewhere in this disclosure or priority-linked disclosures can be applied to this example where relevant.

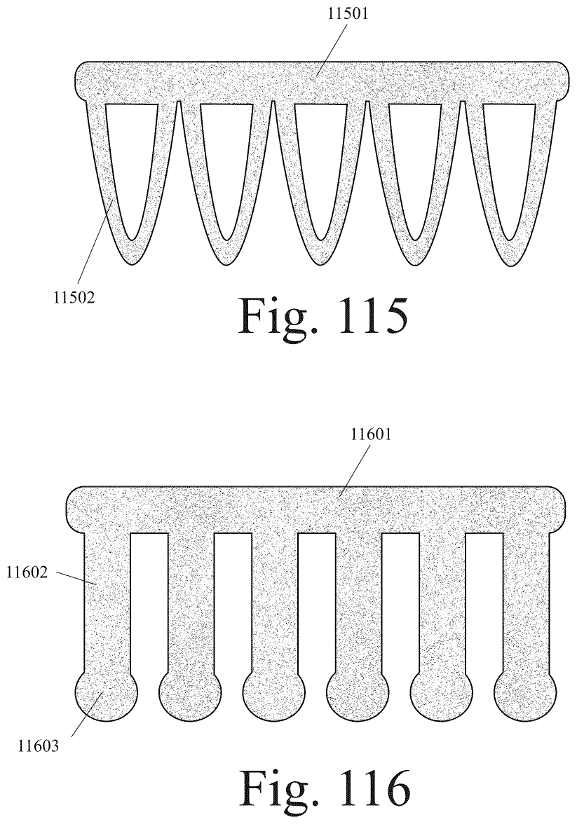

FIG. 114 shows a side cross-sectional view of an EEG electrode for use on a hair-covered portion of a person's head comprising: a (disk-shaped) distal portion 11401; and a plurality of paraboloid-shaped proximal protrusions, including 11402 and 11403, which extend out from the distal portion toward the person's head in order to penetrate between hairs and come into contact with the surface of the person's head. In this example, there are three protrusions in a side cross-section of the electrode. Example variations discussed elsewhere in this disclosure or priority-linked disclosures can be applied to this example where relevant.

FIG. 115 shows a side cross-sectional view of an EEG electrode for use on a hair-covered portion of a person's head comprising: a (disk-shaped) distal portion 11501; and a plurality of arch-shaped proximal protrusions including 11502 and 11503 which extend out from the distal portion toward the person's head in order to penetrate between hairs and come into contact with the surface of the person's head.

In an example, an arch-shaped protrusion can have a parabolic, conic section, catenary, and/or carlavian curve shape. In an example, an electrode can comprise a distal portion and a plurality of proximal loops which extend out from the distal portion toward the person's head in order to penetrate between hairs and come into contact with the surface of the person's head. In an example, protruding arches and/or loops can be oriented along spokes which radiate from the center of the distal portion. In an example, protruding arches and/or loops can be parallel to each other. In an example, protruding arches and/or loops can be configured in a hexagonal grid. Example variations discussed elsewhere in this disclosure or priority-linked disclosures can be applied to this example where relevant.

FIG. 116 shows a side cross-sectional view of an EEG electrode for use on a hair-covered portion of a person's head comprising: a distal portion 11601; and a plurality of column-and-ball protrusions which extend out from the distal portion toward the person's head in order to penetrate between hairs and come into contact with the surface of the person's head, wherein a column-and-ball protrusion further comprises a distal (farther from the surface of a person's head) column 11602 and a proximal (closer to the surface of the person's head) ball 11603.

In an example, a protrusion can comprise a column with a ball attached to its proximal end, wherein the ball has a diameter which is greater than the diameter of the column. In an example, a column can be a circular column. In an example, the column and ball can comprise a single piece made from the same material. In an example, the column and ball can be different pieces which are attached together and/or made from different materials. In an example, the diameter of a ball portion of a protrusion which is closer to the center of the electrode can be greater than the diameter of a ball portion of a protrusion which is farther from the center of the electrode. In an example, the diameter of a ball portion of a protrusion which is closer to the center of the electrode can be less than the diameter of a ball portion of a protrusion which is farther from the center of the electrode.

In an example, a plurality of column-and-ball protrusions can be configured in two or more nested (e.g. concentric) rings. In an example, a plurality of column-and-ball protrusions can be configured in an orthogonal grid. In an example, column-and-ball protrusions can extend out perpendicularly from a distal portion of an electrode toward the surface of a person's head. Example variations discussed elsewhere in this disclosure or priority-linked disclosures can be applied to this example where relevant.

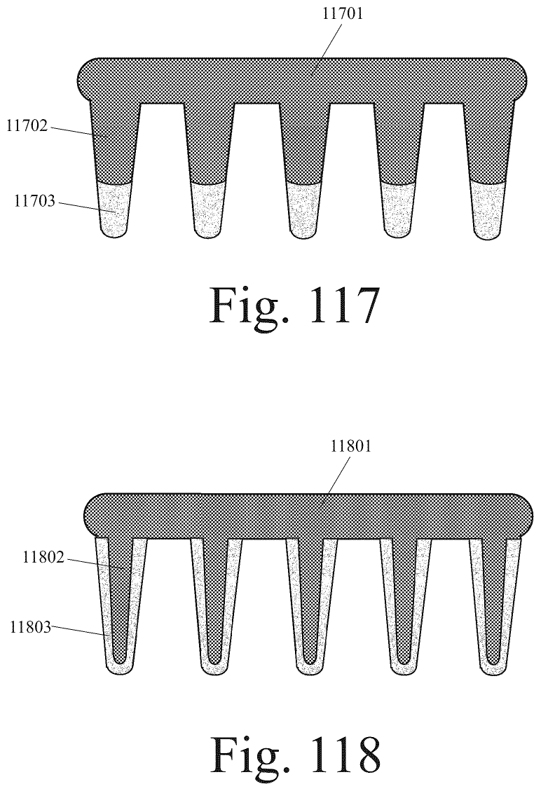

FIG. 117 shows a side cross-sectional view of an EEG electrode for use on a hair-covered portion of a person's head comprising: a distal portion 11701; and a plurality of protrusions which extend out from the distal portion toward the person's head in order to penetrate between hairs and come into contact with the surface of the person's head, wherein a protrusion further comprises a distal (farther from the surface of a person's head) portion 11702 made from a first material and a proximal (closer to the surface of the person's head) portion 11703 made from a second material.

In an example, the distal half of a protrusion is made from a first material and the proximal half of the protrusion is made from a second material. In an example, between half and three-quarters of a protrusion is made from a first material and the rest of the protrusion is made from a second material. In an example, the 50% to 75% of a protrusion which is closest to the distal portion of the electrode is made from a first material and the rest of the protrusion is made from a second material. In an example, a protrusion is made from a first material except for a proximal (closest to the surface of a person's head) tip, peak, vertex, or cap which is made from a second material. In an example, a protrusion is made from a first material except for a coating on the tip, peak, vertex, or cap of the protrusion which is made from a second material.

In an example, the conductivity of the first material is greater than that of the second material. In an example, the durometer of the first material is greater than that of the second material. In an example, the Young's modulus of the first material is greater than that of the second material. In an example, the elasticity of the first material is greater than that of the second material. In an example, the flexibility of the first material is greater than that of the second material. In an example, the conductivity of the first material is less than that of the second material. In an example, the durometer of the first material is less than that of the second material. In an example, the Young's modulus of the first material is less than that of the second material. In an example, the elasticity of the first material is less than that of the second material. In an example, the flexibility of the first material is less than that of the second material.

In an example, the first material is a metal (e.g. silver). In an example, the first material is an inherently-conductive polymer. In an example, the first material is an inherently-nonconductive polymer. In an example, the first material is an inherently-nonconductive polymer (e.g. PDMS) which has been made conductive by doping, impregnation, and/or coating with carbon structures (e.g. carbon nanotubes). In an example, the first material is an inherently-nonconductive polymer (e.g. PDMS) which has been made conductive by doping, impregnation, and/or coating with metal. In an example a first material can have a higher amount of doping or impregnation with metal and/or carbon structures than a second material.

In an example, the second material is a metal (e.g. silver). In an example, the second material is an inherently-conductive polymer. In an example, the second material is an inherently-nonconductive polymer. In an example, the second material is an inherently-nonconductive polymer (e.g. PDMS) which has been made conductive by doping, impregnation, and/or coating with carbon structures (e.g. carbon nanotubes). In an example, the second material is an inherently-nonconductive polymer (e.g. PDMS) which has been made conductive by doping, impregnation, and/or coating with metal. In an example a second material can have a higher amount of doping or impregnation with metal and/or carbon structures than a first material.

In an example, the proportion of the first material vs. the second material in a protrusion can vary with distance from the center of the electrode. In an example, a protrusion which is closer to the center of the electrode can have a greater percentage of a first material than a protrusion which is farther from the center. In an example, a protrusion which is closer to the center of the electrode can have a lower percentage of a first material than a protrusion which is farther from the center. Example variations discussed elsewhere in this disclosure or priority-linked disclosures can be applied to this example where relevant.

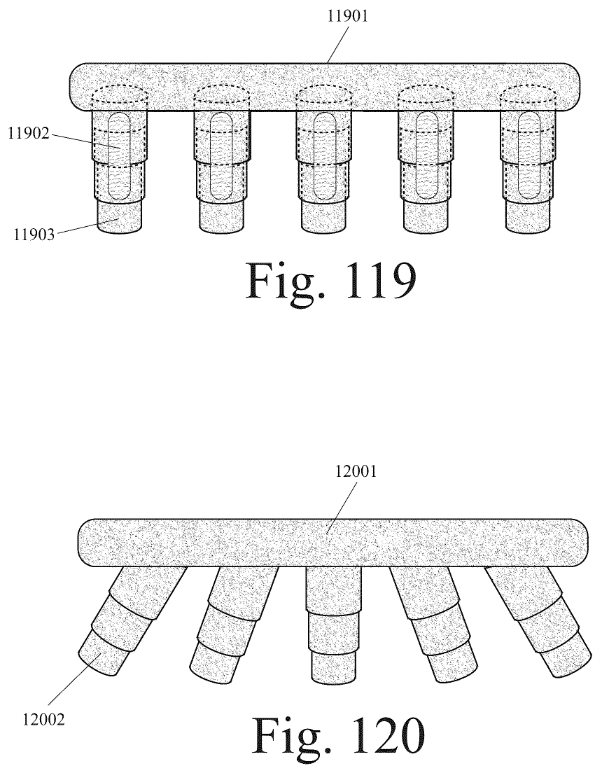

FIG. 118 shows a side cross-sectional view of an EEG electrode for use on a hair-covered portion of a person's head comprising: a distal portion 11801; and a plurality of protrusions which extend out from the distal portion toward the person's head in order to penetrate between hairs and come into contact with the surface of the person's head, wherein a protrusion further comprises a inner core 11802 made from a first material and an outer layer 11803 made from a second material.

In an example, an inner core of a protrusion spans between 20% and 60% of a cross-sectional area of a protrusion. In an example, an inner core of a protrusion spans between 40% and 80% of a cross-sectional area of the protrusion. In an example an inner core extends all the way from the distal portion an electrode to the proximal tip, peak, vertex, or cap of a protrusion. In an example an inner core spans between 50% and 95% of the longitudinal axis of a protrusion. In an example, an inner core can be a cylindrical core within a frustum-shape protrusion. In an example, an inner core can be a frustum-shaped core within a frustum-shape protrusion. In an example, an inner core can be a cylindrical core within a cylindrical protrusion.

In an example, the conductivity of the first material is greater than that of the second material. In an example, the durometer of the first material is greater than that of the second material. In an example, the Young's modulus of the first material is greater than that of the second material. In an example, the elasticity of the first material is greater than that of the second material. In an example, the flexibility of the first material is greater than that of the second material. In an example an inner core is fluid. In an example an inner core is a conductive fluid. In an example, the conductivity of the first material is less than that of the second material. In an example, the durometer of the first material is less than that of the second material. In an example, the Young's modulus of the first material is less than that of the second material. In an example, the elasticity of the first material is less than that of the second material. In an example, the flexibility of the first material is less than that of the second material.

In an example, an inner core of a protrusion can be stiffer and/or more resilient than an outer layer of a protrusion. In an example, an outer layer of a protrusion can more compliant or compressible than an inner core of a protrusion. In an example, an inner core can be made with metal and an outer layer can be made with a conductive polymer. In an example, an inner core can be made with metal and an outer layer can be made with an inherently-nonconductive polymer (e.g. PDMS) which has been made conductive by doping, impregnation, and/or coating with carbon structures (e.g. carbon nanotubes) and/or metal.

In an example, the first material is a metal (e.g. silver). In an example, the first material is an inherently-conductive polymer. In an example, the first material is an inherently-nonconductive polymer. In an example, the first material is an inherently-nonconductive polymer (e.g. PDMS) which has been made conductive by doping, impregnation, and/or coating with carbon structures (e.g. carbon nanotubes). In an example, the first material is an inherently-nonconductive polymer (e.g. PDMS) which has been made conductive by doping, impregnation, and/or coating with metal. In an example a first material can have a higher amount of doping or impregnation with metal and/or carbon structures than a second material.

In an example, the second material is a metal (e.g. silver). In an example, the second material is an inherently-conductive polymer. In an example, the second material is an inherently-nonconductive polymer. In an example, the second material is an inherently-nonconductive polymer (e.g. PDMS) which has been made conductive by doping, impregnation, and/or coating with carbon structures (e.g. carbon nanotubes). In an example, the second material is an inherently-nonconductive polymer (e.g. PDMS) which has been made conductive by doping, impregnation, and/or coating with metal. In an example a second material can have a higher amount of doping or impregnation with metal and/or carbon structures than a first material.

In an example, the size and/or shape of an inner core can vary with distance from the center of the electrode. In an example, a protrusion which is closer to the center of the electrode can have a larger inner core than a protrusion which is farther from the center. In an example, a protrusion which is closer to the center of the electrode can have a smaller inner core than a protrusion which is farther from the center. Example variations discussed elsewhere in this disclosure or priority-linked disclosures can be applied to this example where relevant.

FIG. 119 shows a side cross-sectional view of an EEG electrode for use on a hair-covered portion of a person's head comprising: a distal portion 11901; and a plurality of telescoping conductive protrusions which extend out from the distal portion toward the person's head in order to penetrate between hairs and come into contact with the surface of the person's head, wherein a telescoping conductive protrusion further comprises a inner compressible portion 11902 and an outer sequence of telescoping segments 11903. In this example, the protrusions extend out from the distal portion in a perpendicular manner.

In an example, the outer sequence of telescoping segments can be nested (e.g. concentric) cylinders. In an example, the outer sequence of telescoping segments can be nested (e.g. concentric) segments with different diameters. In an example, the outer sequence of telescoping segments can comprise two telescoping segments with different diameters. In an example, the outer sequence of telescoping segments can comprise three or more telescoping segments with different diameters. In an example, telescoping segments can have equal lengths. In an example, proximal (closer to the surface of a person's head) telescoping segments can be shorter than distal (farther from the surface of a person's head) telescoping segments.

In an example, an inner compressible portion can be a chamber which contains a gas. In an example, the amount of contact and/or pressure between protrusions and the surface of a person's head can be adjusted by changing the pressure of a gas inside such a chamber. In an example, an inner compressible portion can be a spring and/or coil. In an example, the amount of contact and/or pressure between protrusions and the surface of a person's head can be adjusted by rotating such a spring and/or coil. In an example, an inner compressible portion can be made from compressible (e.g. low durometer) foam. Example variations discussed elsewhere in this disclosure or priority-linked disclosures can be applied to this example where relevant.

FIG. 120 shows a side cross-sectional view of an EEG electrode for use on a hair-covered portion of a person's head comprising: a distal portion 12001; and a plurality of conductive protrusions which extend out from the distal portion toward the person's head in order to penetrate between hairs and come into contact with the surface of the person's head, wherein a conductive protrusion further comprises a inner compressible portion 12002 and an outer sequence of telescoping segments 12003. In this example, protrusions extend out from the distal portion at different angles. In this example, the angles at which protrusions extend out from the distal portion vary with the distance of a protrusion from the center of the electrode. In an example, protrusions which are closer to the center of the electrode can be more perpendicular than protrusions which are farther from the center of the electrode. In an example, protrusions which are farther from the center of the electrode can tilt, point, bow, or curve away from the center of the electrode.

In an example, the outer sequence of telescoping segments can be nested (e.g. concentric) cylinders. In an example, the outer sequence of telescoping segments can be nested (e.g. concentric) segments with different diameters. In an example, the outer sequence of telescoping segments can comprise two telescoping segments with different diameters. In an example, the outer sequence of telescoping segments can comprise three or more telescoping segments with different diameters. In an example, telescoping segments can have equal lengths. In an example, proximal (closer to the surface of a person's head) telescoping segments can be shorter than distal (farther from the surface of a person's head) telescoping segments.

In an example, an inner compressible portion can be a chamber which contains a gas. In an example, the amount of contact and/or pressure between protrusions and the surface of a person's head can be adjusted by changing the pressure of a gas inside such a chamber. In an example, an inner compressible portion can be a spring and/or coil. In an example, the amount of contact and/or pressure between protrusions and the surface of a person's head can be adjusted by rotating such a spring and/or coil. In an example, an inner compressible portion can be made from compressible (e.g. low durometer) foam. Example variations discussed elsewhere in this disclosure or priority-linked disclosures can be applied to this example where relevant.

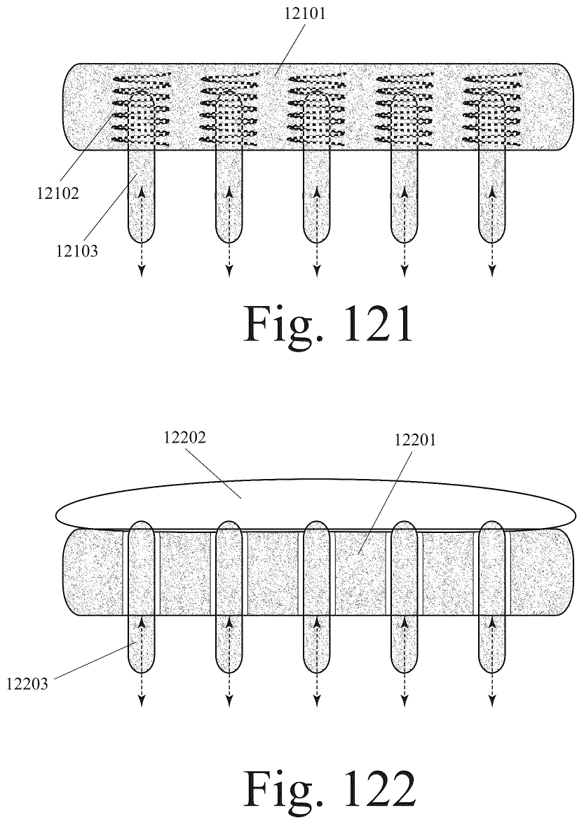

FIG. 121 shows a side cross-sectional view of an EEG electrode for use on a hair-covered portion of a person's head comprising: a distal portion 12101; a plurality of movable conductive protrusions, including 12103, which extend out from the distal portion toward the person's head in order to penetrate between hairs and come into contact with the surface of the person's head; and a plurality of springs (or coils), including 12102, which compel protrusions in the plurality of movable conductive protrusions toward the surface of the person's head.

In this example, there is a spring (or coil) for each protrusion. This enables independent movement of different protrusions. In this example, springs (or coils) are within the distal portion of the electrode. Alternatively, springs (or coils) can be external to the distal portion of the electrode. In this example, protrusions extend out in a perpendicular manner from the distal portion. In another example, the angles at which protrusions extend out from the distal portion can vary with the distance of protrusions from the center of the electrode. In an example, a spring (or coil) can encircle a distal portion of a protrusion. In an example, a spring (or coil) can press against the distal end of a protrusion.

In an example, the size and shape of springs (or coils) can vary with distance from the center of the electrode. In an example, a spring (or coil) which is connected to a protrusion which is closer to the center of the electrode can be larger (e.g. larger, stronger, and/or more resilient) than a spring which is connected to a protrusion which is farther from the center of the electrode. In an example, a spring (or coil) which is connected to a protrusion which is father from the center of the electrode can be larger (e.g. larger, stronger, and/or more resilient) than a spring which is connected to a protrusion which is closer to the center of the electrode. Example variations discussed elsewhere in this disclosure or priority-linked disclosures can be applied to this example where relevant.

FIG. 122 shows a side cross-sectional view of an EEG electrode for use on a hair-covered portion of a person's head comprising: a distal portion 12201; a plurality of movable conductive protrusions, including 12203, which extend out from the distal portion toward the person's head in order to penetrate between hairs and come into contact with the surface of the person's head; and an expandable gas-filled or fluid-filled chamber 12202 which compels protrusions in the plurality of movable conductive protrusions toward the surface of the person's head as the chamber is expanded.

In an example, the amount of pressure exerted by protrusions on the surface of a person's head can be adjusted by changing the degree to which the expandable chamber is filled with a gas or liquid. In an example, the amount of pressure exerted by protrusions on the surface of a person's head can be adjusted by changing the pressure of a gas or liquid within the expandable chamber. In this example, an expandable chamber is on the distal side of the distal portion and protrusions extend through the distal portion in order to contact the expandable chamber. In an alternative example, an expandable chamber can be on the proximal side of the distal portion and protrusions need not extend through the distal portion.

In an example, an expandable chamber can be inflated by being filled with a gas (such as air). In an example, the expandable chamber can be a balloon. In an example, a chamber can be expanded by being filled with a liquid. In this example, an expandable chamber is in contact with all of the protrusions and moves all of the protrusions when it is expanded. In an alternative example, there can be a plurality of expandable chambers, wherein each chamber is in contact with one protrusion; this would enable independent movement of different protrusions.

In an example, expansion of a chamber with a gas of liquid can be done manually by the person wearing the device by using a manual air or liquid pump. In an example, this device can further comprise an electromagnetic motor and pump which automatically pumps air or liquid into a chamber. In an example, this device can further comprise an electromagnetic motor and pump which automatically pumps air or liquid into a chamber until a desired pressure level is reached. In an example, this device can further comprise one or more pressure sensors on one or more protrusions, wherein an automatic pump adjusts the pressure inside one or more expandable chambers in order to adjust the pressure levels between protrusions and the surface of a person's head. Pressure levels can also be automatically adjusted in order to achieve desired levels of conductivity between protrusions and the surface of a person's head. In an example, there can be a separate pneumatic or hydraulic mechanism for each protrusion which selectively and independently adjusts the degree of pressure between each protrusion and the surface of a person's head. Example variations discussed elsewhere in this disclosure or priority-linked disclosures can be applied to this example where relevant.

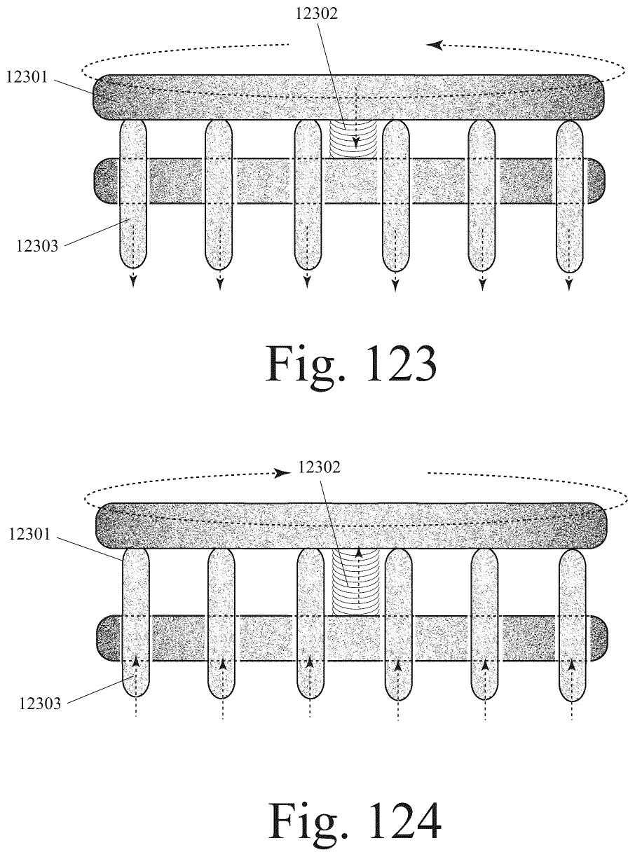

FIGS. 123 and 124 show two side cross-sectional views, at two different times, of an EEG electrode for use on a hair-covered portion of a person's head comprising: a distal portion 12301; a plurality of movable conductive protrusions, including 12303, which extend toward the person's head in order to penetrate between hairs and come into contact with the surface of the person's head; and a (helical) threaded mechanism 12302 which is connected to the distal portion, wherein rotation of the distal portion rotates the threaded mechanism, rotation of the thread mechanism moves the distal portion toward the surface of the person's head, and movement of the distal portion toward the surface of the person's head increases the pressure exerted by protrusions (in plurality of movable conductive protrusions) against the surface of the person's head.

FIG. 123 shows this electrode at a first time wherein rotation of the distal portion in a first direction (e.g. counter-clockwise) moves the distal portion closer toward the surface of the person's head and increases contact and/or pressure between the protrusions and the surface of the person's head. FIG. 124 shows this electrode at a second time wherein rotation of the distal portion in a second direction (e.g. clockwise) moves the distal portion farther from the surface of the person's head and decreases contact and/or pressure between the protrusions and the surface of the person's head.

In an example, rotation of the distal portion can be done manually. In an example, this device can further comprise an actuator which automatically rotates the distal portion. In an example, the device can further comprise one or more pressure sensors on one or more protrusions, wherein these pressure sensors measure pressure levels between protrusions and the surface of a person's head. In an example, an actuator can automatically rotate the distal portion in order to achieve desired levels of contact, pressure, and/or conductivity between protrusions and the surface of a person's head. Example variations discussed elsewhere in this disclosure or priority-linked disclosures can be applied to this example where relevant.

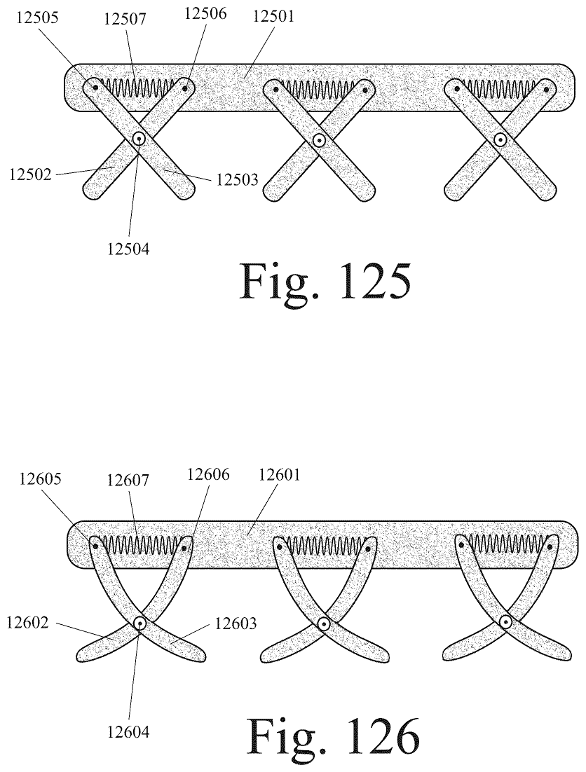

FIG. 125 shows a cross-sectional view of an EEG electrode for use on a hair-covered portion of a person's head comprising: (a) a distal portion 12501; and (b) a plurality of articulated conductive protrusions which extend out from the distal portion toward the person's head in order to penetrate between hairs and come into contact with the surface of the person's head; wherein each protrusion further comprises a first longitudinal segment 12502 (e.g. segment, section, bar, prong, or arm) which is movably attached to the distal portion by a first connection, joint, or axle 12506; wherein each protrusion further comprises a second longitudinal segment 12503 (e.g. segment, section, bar, prong, or arm) which is movably attached to the distal portion by a second connection, joint, or axle 12505; and wherein the first and second longitudinal segments are movably connected to each other by a third connection, joint, or axle 12504. In an example, a protrusion can further comprise a spring or elastic band 12507 between the first and second longitudinal segments.

In an example, movement of a second longitudinal segment (e.g. segment, section, bar, prong, or arm) relative to a first longitudinal segment (e.g. segment, section, bar, prong, or arm), or vice versa, changes the distance by which a protrusion extends out from the distal portion, thereby changing the degree of contact and/or pressure between the protrusion and the surface of the person's head. In an example, rotation, tilting, or pivoting of a second longitudinal segment relative to a first longitudinal segment, or vice versa, around the third connection, joint, or axle changes the extension distance of a protrusion, thereby changing the degree of contact and/or pressure between the protrusion and the surface of the person's head. In an example, scissor movement of the second longitudinal segment relative to the first longitudinal segment, or vice versa, around the third connection, joint, or axle changes the extension distance of the protrusion, thereby changing the degree of contact and/or pressure between the protrusion and the surface of the person's head.

In this example, longitudinal axes of first and second longitudinal segments (e.g. segments, sections, bars, prongs, or arms) are straight. In another example, longitudinal axes of the first and second longitudinal segments (e.g. segments, sections, bars, prongs, or arms) can be arcuate. In another example, longitudinal axes of the first and second longitudinal segments (e.g. segments, sections, bars, prongs, or arms) can bow outward, away from each other. In another example, longitudinal axes of the first and second longitudinal segments (e.g. segments, sections, bars, prongs, or arms) can bow inward, toward each other. In an example, first and second longitudinal segments can move relative to each other like the two articulated segments of a pair of scissors.

In an example, the distal ends of first and second longitudinal segments can be connected to each other by a spring, elastic band, or other tensile member which draws the ends together and/or pushes the ends apart. In an example, portions of first and second longitudinal segments which are distal (farther from the surface of a person's head) relative to a third connection can be connected to each other by a spring, elastic band, or other tensile member which draws the ends together and/or pushes the ends apart. In an example, portions of first and second longitudinal segments which are proximal (closer to the surface of a person's head) relative to a third connection can be connected to each other by a spring, elastic band, or other tensile member which draws the ends together and/or pushes the ends apart. In an example, contact and/or pressure between the proximal ends of longitudinal segments and the surface of a person's head can cause contraction (or expansion) of the spring, elastic band, or other tensile member. Example variations discussed elsewhere in this disclosure or priority-linked disclosures can be applied to this example where relevant.

FIG. 126 shows a cross-sectional view of an EEG electrode for use on a hair-covered portion of a person's head comprising: (a) a distal portion 12601; and (b) a plurality of articulated conductive protrusions which extend out from the distal portion toward the person's head in order to penetrate between hairs and come into contact with the surface of the person's head; wherein each protrusion further comprises a first longitudinal segment 12602 (e.g. segment, section, bar, prong, or arm) which is movably attached to the distal portion by a first connection, joint, or axle 12606; wherein each protrusion further comprises a second longitudinal segment 12603 (e.g. segment, section, bar, prong, or arm) which is movably attached to the distal portion by a second connection, joint, or axle 12605; and wherein the first and second longitudinal segments are movably connected to each other by a third connection, joint, or axle 12604. In an example, a protrusion can further comprise a spring or elastic band 12607 between the first and second longitudinal segments.

In an example, movement of a second longitudinal segment (e.g. segment, section, bar, prong, or arm) relative to a first longitudinal segment (e.g. segment, section, bar, prong, or arm), or vice versa, changes the distance by which a protrusion extends out from the distal portion, thereby changing the degree of contact and/or pressure between the protrusion and the surface of the person's head. In an example, rotation, tilting, or pivoting of a second longitudinal segment relative to a first longitudinal segment, or vice versa, around the third connection, joint, or axle changes the extension distance of a protrusion, thereby changing the degree of contact and/or pressure between the protrusion and the surface of the person's head. In an example, scissor movement of the second longitudinal segment relative to the first longitudinal segment, or vice versa, around the third connection, joint, or axle changes the extension distance of the protrusion, thereby changing the degree of contact and/or pressure between the protrusion and the surface of the person's head.

In this example, longitudinal axes of first and second longitudinal segments (e.g. segments, sections, bars, prongs, or arms) are arcuate. In this example, longitudinal axes of the first and second longitudinal segments (e.g. segments, sections, bars, prongs, or arms) can bow inward, toward each other. In an example, first and second longitudinal segments can move relative to each other like the two articulated segments of a pair of scissors.

In an example, the distal ends of first and second longitudinal segments can be connected to each other by a spring, elastic band, or other tensile member which draws the ends together and/or pushes the ends apart. In an example, portions of first and second longitudinal segments which are distal (farther from the surface of a person's head) relative to a third connection can be connected to each other by a spring, elastic band, or other tensile member which draws the ends together and/or pushes the ends apart. In an example, portions of first and second longitudinal segments which are proximal (closer to the surface of a person's head) relative to a third connection can be connected to each other by a spring, elastic band, or other tensile member which draws the ends together and/or pushes the ends apart. In an example, contact and/or pressure between the proximal ends of longitudinal segments and the surface of a person's head can cause contraction (or expansion) of the spring, elastic band, or other tensile member. Example variations discussed elsewhere in this disclosure or priority-linked disclosures can be applied to this example where relevant.

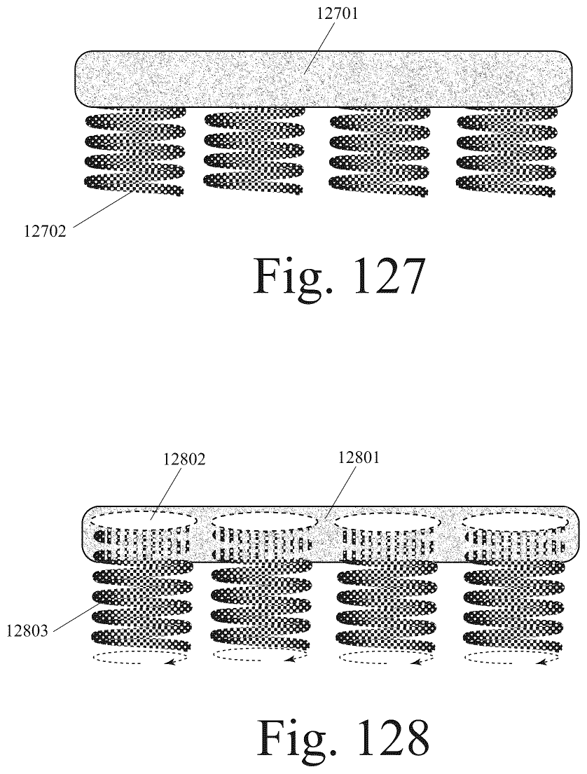

FIG. 127 shows a cross-sectional view of an EEG electrode for use on a hair-covered portion of a person's head comprising: a distal portion 12701; and a plurality of helical conductive protrusions, including 12702, which extend out from the distal portion toward the person's head in order to penetrate between hairs and come into contact with the surface of the person's head. In an example, there can also be a soft sphere (e.g. ball) at the end of a helical conductive protrusion.

In an example, a distal portion of an electrode can have a circular and/or disk shape. In an example, a distal portion can have an oblong, oval, or elliptical shape. In an example, a distal portion can have a square or rectangular shape. In an example, a distal portion can have a rounded square or rounded rectangular shape. In an example, a distal portion can have a hexagonal shape. In an example, the distal portion of the electrode can have non-uniform thickness. In an example, the center of a distal portion can be thicker than the periphery of the distal portion. In an example, the center of a distal portion can be thinner than the periphery of the distal portion.

In an example, a helical conductive protrusion can be a spring or coil. In an example, an electrode can comprise two or more nested (e.g. concentric) rings of helical conductive protrusions. In an example, an electrode can comprise two or more rows and columns of helical conductive protrusions. In an example, the longitudinal axis of a helical conductive protrusion can be substantially perpendicular to the best-fitting plane of the distal portion of an electrode. In an example, the diameter of a helical conductive protrusion can vary with distance from the distal portion. In an example, the diameter of a helical conductive protrusion can decrease with distance from the distal portion. In an example, the diameter of a helical conductive protrusion can increase with distance from the distal portion.

In an example, the angle between a longitudinal axis of a helical conductive protrusion and the best-fitting plan of a distal portion can vary with the distance of the protrusion from the center of the distal portion. In an example, the angle between a longitudinal axis of a helical conductive protrusion and the best-fitting plan of a distal portion can increase with the distance of the protrusion from the center of the distal portion. In an example, the angle between a longitudinal axis of a helical conductive protrusion and the best-fitting plan of a distal portion can decrease with the distance of the protrusion from the center of the distal portion.

In an example, a helical conductive protrusion can be a metal spring or coil. In an example, a helical conductive protrusion can be a conductive-polymer spring or coil. In an example, a helical conductive protrusion can be made from an inherently-nonconductive polymer (e.g. PDMS) which has been made conductive by doping, impregnation, and/or coating with carbon structures (e.g. carbon nanotubes). In an example, a helical conductive protrusion can be made from metal and an inherently-conductive polymer.

In an example, a helical conductive protrusion can be made from metal and an inherently-nonconductive polymer. In an example, a helical conductive protrusion can be made from metal and an inherently-nonconductive polymer (e.g. PDMS) which has been made conductive by doping, impregnation, and/or coating with carbon structures (e.g. carbon nanotubes). In an example, a helical conductive protrusion can have a metal core and a conductive-polymer outer layer (or coating). Example variations discussed elsewhere in this disclosure or priority-linked disclosures can be applied to this example where relevant.

FIG. 128 shows a cross-sectional view of an EEG electrode for use on a hair-covered portion of a person's head comprising: a distal portion 12801; a plurality of helical conductive protrusions, including protrusion 12803, which extend out from the distal portion toward the person's head in order to penetrate between hairs and come into contact with the surface of the person's head; and a plurality of actuators, including actuator 12802, which rotate helical conductive protrusions in the plurality of helical conductive protrusions. In an example, there can also be a soft sphere (e.g. ball) at the end of a helical conductive protrusion. In an example, rotation of a helical conductive protrusion enhances the ability of the protrusion to penetrate between hairs and come into contact with the surface of the person's head, but not penetrate or abrade the person's skin.

In an example, a distal portion of an electrode can have a circular and/or disk shape. In an example, a distal portion can have an oblong, oval, or elliptical shape. In an example, a distal portion can have a square or rectangular shape. In an example, a distal portion can have a rounded square or rounded rectangular shape. In an example, a distal portion can have a hexagonal shape. In an example, the distal portion of the electrode can have non-uniform thickness. In an example, the center of a distal portion can be thicker than the periphery of the distal portion. In an example, the center of a distal portion can be thinner than the periphery of the distal portion.

In an example, a helical conductive protrusion can be a spring or coil. In an example, an electrode can comprise two or more nested (e.g. concentric) rings of helical conductive protrusions. In an example, an electrode can comprise two or more rows and columns of helical conductive protrusions. In an example, the longitudinal axis of a helical conductive protrusion can be substantially perpendicular to the best-fitting plane of the distal portion of an electrode. In an example, the diameter of a helical conductive protrusion can vary with distance from the distal portion. In an example, the diameter of a helical conductive protrusion can decrease with distance from the distal portion. In an example, the diameter of a helical conductive protrusion can increase with distance from the distal portion.

In an example, the angle between a longitudinal axis of a helical conductive protrusion and the best-fitting plan of a distal portion can vary with the distance of the protrusion from the center of the distal portion. In an example, the angle between a longitudinal axis of a helical conductive protrusion and the best-fitting plan of a distal portion can increase with the distance of the protrusion from the center of the distal portion. In an example, the angle between a longitudinal axis of a helical conductive protrusion and the best-fitting plan of a distal portion can decrease with the distance of the protrusion from the center of the distal portion.

In an example, a helical conductive protrusion can be a metal spring or coil. In an example, a helical conductive protrusion can be a conductive-polymer spring or coil. In an example, a helical conductive protrusion can be made from an inherently-nonconductive polymer (e.g. PDMS) which has been made conductive by doping, impregnation, and/or coating with carbon structures (e.g. carbon nanotubes). In an example, a helical conductive protrusion can be made from metal and an inherently-conductive polymer. In an example, a helical conductive protrusion can be made from metal and an inherently-nonconductive polymer. In an example, a helical conductive protrusion can be made from metal and an inherently-nonconductive polymer (e.g. PDMS) which has been made conductive by doping, impregnation, and/or coating with carbon structures (e.g. carbon nanotubes). In an example, a helical conductive protrusion can have a metal core and a conductive-polymer outer layer (or coating).

In an example, an actuator can be a small electromagnetic motor. In an example, an actuator can be within the distal portion of the electrode. In an example, an actuator can be between the distal portion of the electrode and a helical conductive protrusion which extends out from the electrode. In an example, an actuator can rotate a helical conductive protrusion in a clockwise (or counter-clockwise) direction around the central longitudinal axis of the protrusion. In an example, there can be one actuator for each protrusion. Example variations discussed elsewhere in this disclosure or priority-linked disclosures can be applied to this example where relevant.

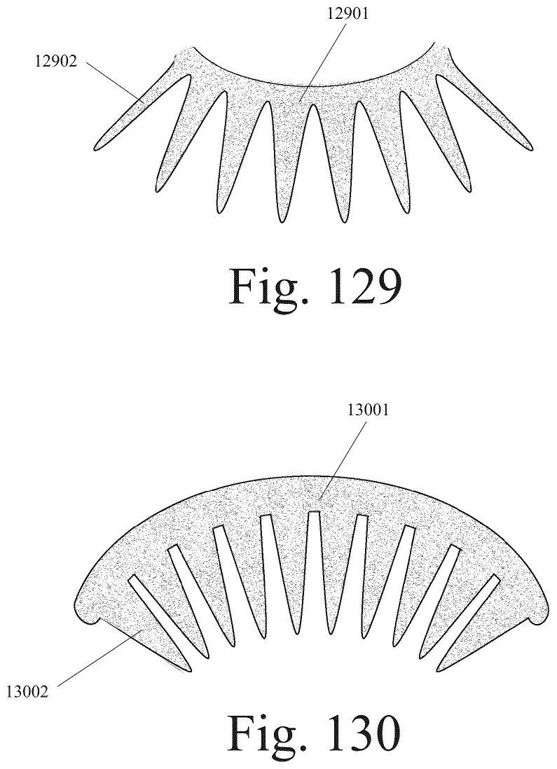

FIG. 129 shows a cross-sectional view of an EEG electrode for use on a hair-covered portion of a person's head comprising: a convex distal portion 12901 whose center curves toward the surface of a person's head and whose perimeter curves away from the surface of the person's head; a plurality of protrusions (e.g. prongs, teeth, spikes, fingers, and/or protrusions), including protrusion 12902, which extend out from the distal portion toward the person's head in order to penetrate between hairs and come into contact with the surface of the person's head.

In an example, a protrusion can have a sinusoidal shape. In an example, a protrusion can be shaped like three or four phases of a four-phase sine wave. In an example, a plurality of protrusions can be shaped like a sine wave. In an example, a plurality of protrusions have the shape of a sine wave along a curved line. In an example, a plurality of protrusions have the shape of a sine wave along an arc. In an example, a plurality of protrusions have the shape of a sine wave along a conic section. In an example, a plurality of protrusions have the shape of a sine wave along a section of a sphere or ellipse. In an example, an electrode with protrusions can be “shaped like eyelashes”, wherein “shaped like eyelashes” means having a downward convex shape and longitudinal protrusions which extend out radially and downward from the convex shape.

In an example, the angle between a longitudinal axis of a protrusion and the best-fitting plane of the distal portion can vary with the distance of the protrusion from the center of the distal portion. In an example, the angle between a longitudinal axis of a protrusion and the best-fitting plane of the distal portion can increase with the distance of the protrusion from the center of the distal portion. In an example, the angle between a longitudinal axis of a protrusion and the surface of a person's head can vary with the distance of the protrusion from the center of the distal portion. In an example, the angle between a longitudinal axis of a protrusion and the surface of a person's head can increase with the distance of the protrusion from the center of the distal portion. Example variations discussed elsewhere in this disclosure or priority-linked disclosures can be applied to this example where relevant.

FIG. 130 shows a cross-sectional view of an EEG electrode for use on a hair-covered portion of a person's head comprising: a concave distal portion 13001 whose center curves away from the surface of a person's head and whose perimeter curves toward the surface of the person's head; a plurality of protrusions (e.g. prongs, teeth, spikes, fingers, and/or protrusions), including protrusion 13002, which extend out from the distal portion toward the person's head in order to penetrate between hairs and come into contact with the surface of the person's head.

In an example, a protrusion can have a sinusoidal shape. In an example, a protrusion can have a parabolic-shaped cross-section. In an example, a protrusion can have a paraboloidal shape. In an example, a protrusion can be shaped like three or four phases of a four-phase sine wave. In an example, a plurality of protrusions can be shaped like a sine wave. In an example, a plurality of protrusions have the shape of a sine wave along a curved line. In an example, a plurality of protrusions have the shape of a sine wave along an arc. In an example, a plurality of protrusions have the shape of a sine wave along a conic section. In an example, a plurality of protrusions have the shape of a sine wave along a section of a sphere or ellipse. In an example, virtual extensions (in a proximal direction) of the longitudinal axes of a plurality of protrusions can intersect virtually in space. In an example, virtual extensions in a proximal direction of the longitudinal axes of a plurality of protrusions can intersect virtually on or in a person's head.

In an example, the angle between a longitudinal axis of a protrusion and the best-fitting plane of the distal portion can vary with the distance of the protrusion from the center of the distal portion. In an example, the angle between a longitudinal axis of a protrusion and the best-fitting plane of the distal portion can decrease with the distance of the protrusion from the center of the distal portion. In an example, the angle between a longitudinal axis of a protrusion and the best-fitting plane of the distal portion can increase with the distance of the protrusion from the center of the distal portion. In an example, the angle between a longitudinal axis of a protrusion and the surface of a person's head can vary with the distance of the protrusion from the center of the distal portion. In an example, the angle between a longitudinal axis of a protrusion and the surface of a person's head can decrease with the distance of the protrusion from the center of the distal portion. Example variations discussed elsewhere in this disclosure or priority-linked disclosures can be applied to this example where relevant.

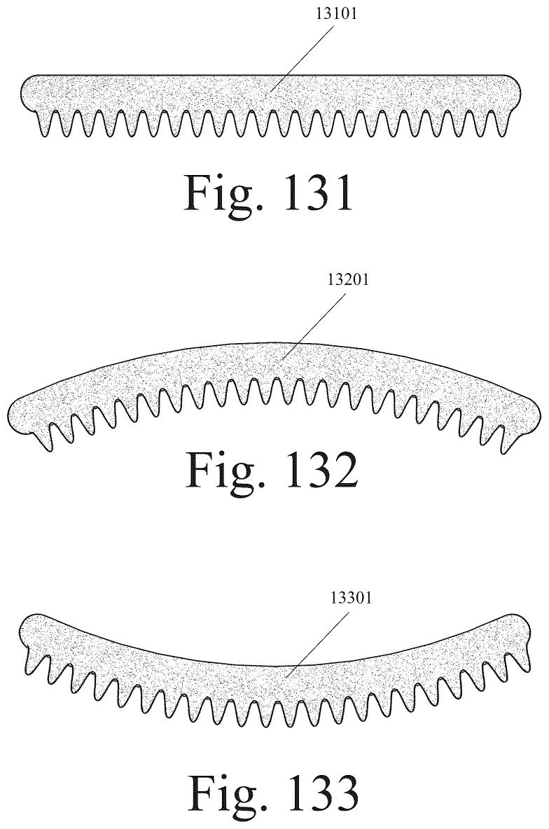

FIG. 131 shows a cross-sectional view of an EEG electrode for use on a hair-covered portion of a person's head comprising: a conductive electrode 13101, wherein the electrode has a lateral cross-sectional shape with a flat distal side and a sinusoidal proximal side, wherein the distal side faces away from the surface of a person's head, wherein the proximal side faces toward the surface of the person's head, and wherein sinusoidal protrusions on the proximal side penetrate between hairs and come into contact with the surface of the person's head. In an example, a longitudinal axis of the sinusoidal proximal side can be substantially parallel to the flat distal side.

In an example, sinusoidal protrusions can be sinusoidal ridges. In an example, a proximal side can comprise a series of sinusoidal ridges which span an electrode laterally. In an example, sinusoidal protrusions can be sinusoidal rings. In an example, a proximal side can comprise a series of nested sinusoidal rings around the center of an electrode. In an example, sinusoidal protrusions can be sinusoidal peaks. In an example, a proximal side of an electrode can have a two-directional sinusoidal surface (e.g. similar to interior of an egg carton).

In an example, a longitudinal axis of the sinusoidal proximal side of an electrode can be substantially parallel to the flat distal side of the electrode. In an example, there can be between 2 and 8 sinusoidal ridges, rings, or peaks on the proximal side of the electrode. In an example, there can be between 5 and 15 sinusoidal ridges, rings, or peaks on the proximal side of the electrode. In an example, there can be between 10 and 30 sinusoidal ridges, rings, or peaks on the proximal side of the electrode. In an example, the amplitude of a sinusoidal wave of ridges, rings, or peaks on the proximal side can between 50% and 80% of the average distance between the proximal and distal sides of the electrode. In an example, the amplitude of a sinusoidal wave of ridges, rings, or peaks on the proximal side can between 100% and 150% of the average distance between the proximal and distal sides of the electrode.

In an example, sinusoidal protrusions on the proximal side of an electrode can all have the same amplitude. In an example, the amplitudes of sinusoidal protrusions on an electrode can vary with distance of protrusions from the center of the electrode. In an example, the amplitudes of sinusoidal protrusions on an electrode can increase with increased distance of the protrusions from the center of the electrode. In an example, the amplitudes of sinusoidal protrusions which are farther from the center of an electrode can be greater than the amplitudes of sinusoidal protrusions which are closer to the center of the electrode. In an example, the amplitudes of sinusoidal protrusions on an electrode can decrease with increased distance of the protrusions from the center of the electrode. In an example, the amplitudes of sinusoidal protrusions which are farther from the center of an electrode can be less than the amplitudes of sinusoidal protrusions which are closer to the center of the electrode.

In an example, sinusoidal protrusions on the proximal side of an electrode can all have the same wavelength. In an example, the wavelengths of sinusoidal protrusions on an electrode can vary with distance of protrusions from the center of the electrode. In an example, the wavelengths of sinusoidal protrusions on an electrode can increase with increased distance of the protrusions from the center of the electrode. In an example, the wavelengths of sinusoidal protrusions which are farther from the center of an electrode can be greater than the wavelengths of sinusoidal protrusions which are closer to the center of the electrode. In an example, the wavelengths of sinusoidal protrusions on an electrode can decrease with increased distance of the protrusions from the center of the electrode. In an example, the wavelengths of sinusoidal protrusions which are farther from the center of an electrode can be less than the wavelengths of sinusoidal protrusions which are closer to the center of the electrode.

In an example, sinusoidal ridges, rings, or peaks can be made from an inherently-nonconductive polymer (e.g. PDMS) which has been made conductive by doping, impregnation, and/or coating with carbon structures (e.g. carbon nanotubes). In an example, sinusoidal ridges, rings, or peaks can be made from an inherently-nonconductive polymer (e.g. PDMS) which has been made conductive by doping, impregnation, and/or coating with metal (e.g. silver). In an alternative example, a proximal side of an electrode can have sawtooth wave shape instead of a sinusoidal wave shape. Example variations discussed elsewhere in this disclosure or priority-linked disclosures can be applied to this example where relevant.

FIG. 132 shows a cross-sectional view of an EEG electrode for use on a hair-covered portion of a person's head comprising: a conductive electrode 13201, wherein the electrode has a lateral cross-sectional shape with a curved distal side whose center bows outward in a distal direction; and a sinusoidal proximal side, wherein the distal side faces away from the surface of a person's head, wherein the proximal side faces toward the surface of the person's head, and wherein sinusoidal protrusions on the proximal side penetrate between hairs and come into contact with the surface of the person's head. In an example, a longitudinal axis of the sinusoidal proximal side can be substantially parallel to the curved distal side. In an example, the center of the electrode can be farther from the surface of the person's head than the periphery (or perimeter) of the electrode.

In an example, sinusoidal protrusions can be sinusoidal ridges. In an example, a proximal side can comprise a series of sinusoidal ridges which span an electrode laterally. In an example, sinusoidal protrusions can be sinusoidal rings. In an example, a proximal side can comprise a series of nested sinusoidal rings around the center of an electrode. In an example, sinusoidal protrusions can be sinusoidal peaks. In an example, a proximal side of an electrode can have a two-directional sinusoidal surface (e.g. similar to interior of an egg carton).

In an example, there can be between 2 and 8 sinusoidal ridges, rings, or peaks on the proximal side of the electrode. In an example, there can be between 5 and 15 sinusoidal ridges, rings, or peaks on the proximal side of the electrode. In an example, there can be between 10 and 30 sinusoidal ridges, rings, or peaks on the proximal side of the electrode. In an example, the amplitude of a sinusoidal wave of ridges, rings, or peaks on the proximal side can between 50% and 80% of the average distance between the proximal and distal sides of the electrode. In an example, the amplitude of a sinusoidal wave of ridges, rings, or peaks on the proximal side can between 100% and 150% of the average distance between the proximal and distal sides of the electrode.

In an example, sinusoidal protrusions on the proximal side of an electrode can all have the same amplitude. In an example, the amplitudes of sinusoidal protrusions on an electrode can vary with distance of protrusions from the center of the electrode. In an example, the amplitudes of sinusoidal protrusions on an electrode can increase with increased distance of the protrusions from the center of the electrode. In an example, the amplitudes of sinusoidal protrusions which are farther from the center of an electrode can be greater than the amplitudes of sinusoidal protrusions which are closer to the center of the electrode. In an example, the amplitudes of sinusoidal protrusions on an electrode can decrease with increased distance of the protrusions from the center of the electrode. In an example, the amplitudes of sinusoidal protrusions which are farther from the center of an electrode can be less than the amplitudes of sinusoidal protrusions which are closer to the center of the electrode.

In an example, sinusoidal protrusions on the proximal side of an electrode can all have the same wavelength. In an example, the wavelengths of sinusoidal protrusions on an electrode can vary with distance of protrusions from the center of the electrode. In an example, the wavelengths of sinusoidal protrusions on an electrode can increase with increased distance of the protrusions from the center of the electrode. In an example, the wavelengths of sinusoidal protrusions which are farther from the center of an electrode can be greater than the wavelengths of sinusoidal protrusions which are closer to the center of the electrode. In an example, the wavelengths of sinusoidal protrusions on an electrode can decrease with increased distance of the protrusions from the center of the electrode. In an example, the wavelengths of sinusoidal protrusions which are farther from the center of an electrode can be less than the wavelengths of sinusoidal protrusions which are closer to the center of the electrode.

In an example, sinusoidal ridges, rings, or peaks can be made from an inherently-nonconductive polymer (e.g. PDMS) which has been made conductive by doping, impregnation, and/or coating with carbon structures (e.g. carbon nanotubes). In an example, sinusoidal ridges, rings, or peaks can be made from an inherently-nonconductive polymer (e.g. PDMS) which has been made conductive by doping, impregnation, and/or coating with metal (e.g. silver). In an alternative example, a proximal side of an electrode can have sawtooth wave shape instead of a sinusoidal wave shape. Example variations discussed elsewhere in this disclosure or priority-linked disclosures can be applied to this example where relevant.

FIG. 133 shows a cross-sectional view of an EEG electrode for use on a hair-covered portion of a person's head comprising: a conductive electrode 13301, wherein the electrode has a lateral cross-sectional shape with a curved distal side whose center bows inward in a proximal direction; and a sinusoidal proximal side, wherein the distal side faces away from the surface of a person's head, wherein the proximal side faces toward the surface of the person's head, and wherein sinusoidal protrusions on the proximal side penetrate between hairs and come into contact with the surface of the person's head. In an example, a longitudinal axis of the sinusoidal proximal side can be substantially parallel to the curved distal side. In an example, the center of the electrode can be closer to the surface of the person's head than the periphery (or perimeter) of the electrode.

In an example, sinusoidal protrusions can be sinusoidal ridges. In an example, a proximal side can comprise a series of sinusoidal ridges which span an electrode laterally. In an example, sinusoidal protrusions can be sinusoidal rings. In an example, a proximal side can comprise a series of nested sinusoidal rings around the center of an electrode. In an example, sinusoidal protrusions can be sinusoidal peaks. In an example, a proximal side of an electrode can have a two-directional sinusoidal surface (e.g. similar to interior of an egg carton).

In an example, there can be between 2 and 8 sinusoidal ridges, rings, or peaks on the proximal side of the electrode. In an example, there can be between 5 and 15 sinusoidal ridges, rings, or peaks on the proximal side of the electrode. In an example, there can be between 10 and 30 sinusoidal ridges, rings, or peaks on the proximal side of the electrode. In an example, the amplitude of a sinusoidal wave of ridges, rings, or peaks on the proximal side can between 50% and 80% of the average distance between the proximal and distal sides of the electrode. In an example, the amplitude of a sinusoidal wave of ridges, rings, or peaks on the proximal side can between 100% and 150% of the average distance between the proximal and distal sides of the electrode.

In an example, sinusoidal protrusions on the proximal side of an electrode can all have the same amplitude. In an example, the amplitudes of sinusoidal protrusions on an electrode can vary with distance of protrusions from the center of the electrode. In an example, the amplitudes of sinusoidal protrusions on an electrode can increase with increased distance of the protrusions from the center of the electrode. In an example, the amplitudes of sinusoidal protrusions which are farther from the center of an electrode can be greater than the amplitudes of sinusoidal protrusions which are closer to the center of the electrode. In an example, the amplitudes of sinusoidal protrusions on an electrode can decrease with increased distance of the protrusions from the center of the electrode. In an example, the amplitudes of sinusoidal protrusions which are farther from the center of an electrode can be less than the amplitudes of sinusoidal protrusions which are closer to the center of the electrode.

In an example, sinusoidal protrusions on the proximal side of an electrode can all have the same wavelength. In an example, the wavelengths of sinusoidal protrusions on an electrode can vary with distance of protrusions from the center of the electrode. In an example, the wavelengths of sinusoidal protrusions on an electrode can increase with increased distance of the protrusions from the center of the electrode. In an example, the wavelengths of sinusoidal protrusions which are farther from the center of an electrode can be greater than the wavelengths of sinusoidal protrusions which are closer to the center of the electrode. In an example, the wavelengths of sinusoidal protrusions on an electrode can decrease with increased distance of the protrusions from the center of the electrode. In an example, the wavelengths of sinusoidal protrusions which are farther from the center of an electrode can be less than the wavelengths of sinusoidal protrusions which are closer to the center of the electrode.

In an example, sinusoidal ridges, rings, or peaks can be made from an inherently-nonconductive polymer (e.g. PDMS) which has been made conductive by doping, impregnation, and/or coating with carbon structures (e.g. carbon nanotubes). In an example, sinusoidal ridges, rings, or peaks can be made from an inherently-nonconductive polymer (e.g. PDMS) which has been made conductive by doping, impregnation, and/or coating with metal (e.g. silver). In an alternative example, a proximal side of an electrode can have sawtooth wave shape instead of a sinusoidal wave shape. Example variations discussed elsewhere in this disclosure or priority-linked disclosures can be applied to this example where relevant.

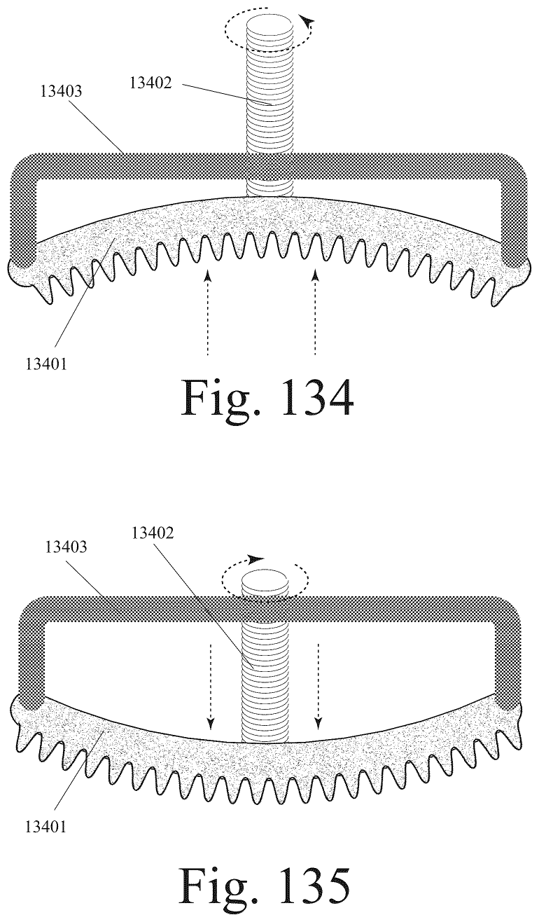

FIGS. 134 and 135 show two cross-sectional views, at two different times, of an EEG electrode for use on a hair-covered portion of a person's head comprising: a distal portion 13403, a proximal portion 13401, wherein the proximal portion is attached to the distal portion, wherein protrusions extend out from the proximal portion to penetrate between hairs and come into contact with the surface of the person's head, and wherein distal means farther from the surface of a person's head and proximal means closer to the surface of the person's head; and a threaded connector 13402 between the distal portion and the proximal portion, wherein rotation of the threaded connector or rotation of the distal portion around the threaded connector changes the concavity or convexity of the proximal portion and changes the level of contact and/or pressure between the protrusions and the surface of the person's head. In an example, the distal portion can be stiffer and/or less flexible than the proximal portion.

FIG. 134 shows this electrode at a first time wherein rotation of the threaded connector in a first direction (e.g. counter-clockwise) has pulled the center of the proximal portion closer toward the distal portion, thereby making the proximal portion more convex relative to the surface of the person's head. FIG. 135 shows this electrode at a second time wherein rotation of the threaded connector in a second direction (e.g. clockwise) has pushed the center of the proximal portion farther from the distal portion, thereby making the proximal portion more concave relative to the surface of the person's head. Example variations discussed elsewhere in this disclosure or priority-linked disclosures can be applied to this example where relevant.

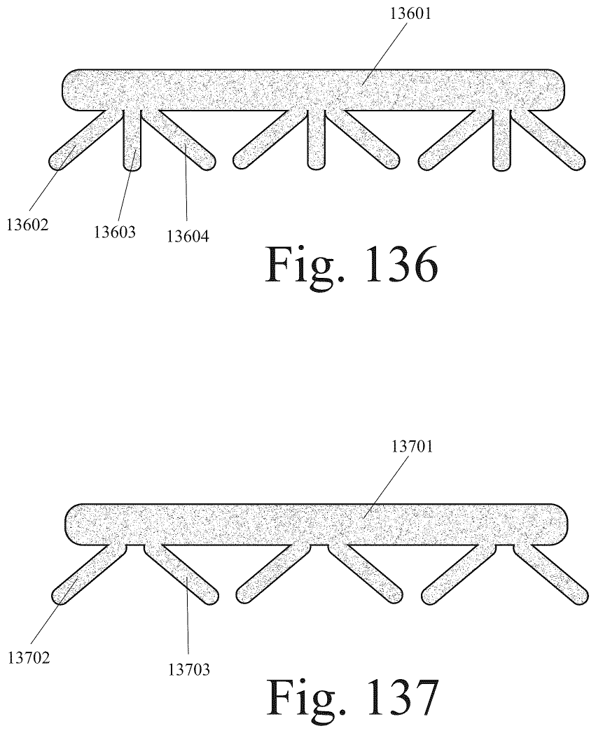

FIG. 136 shows a cross-sectional view of an EEG electrode for use on a hair-covered portion of a person's head comprising: a distal portion 13601; and a plurality of sets of electroconductive proximal protrusions (e.g. pins, prongs, teeth, spikes, fingers, and/or protrusions) which extend out from the distal portion toward the person's head in order to penetrate between hairs and come into contact with the surface of the person's head; wherein (with the possible exception of a set of electroconductive proximal protrusions at the center of the electrode) a set of electroconductive proximal protrusions further comprises: at least one proximal protrusion 13602 whose proximal end tilts (e.g. is angled) away from a central proximal-to-distal axis of the electrode; at least one proximal protrusion 13603 which is perpendicular to the (lateral plane of the) distal portion; and at least one proximal protrusion 13604 whose proximal end tilts (e.g. is angled) toward the central proximal-to-distal axis of the electrode.

In an example, there can be three protrusions in a set. In an example, a set of electroconductive proximal protrusions comprise three protrusions whose proximal ends are at the vertexes of a virtual equilateral triangle. In an example, there can be four protrusions in a set. In an example, a set of electroconductive proximal protrusions comprise four protrusions whose proximal ends are at the vertexes of a virtual square. In an example, there can be six protrusions in a set. In an example, a set of electroconductive proximal protrusions comprise six protrusions whose proximal ends are at the vertexes of a virtual hexagon. In an example, there can be eight protrusions in a set. In an example, a set of electroconductive proximal protrusions comprise eight protrusions whose proximal ends are at the vertexes of a virtual octagon.

In an example, a set of electroconductive proximal protrusions can comprise at least three protrusions, including: at least one protrusion which tilts (e.g. is angled) away from a central proximal-to-distal axis of the electrode, at least one protrusion which is parallel to the central proximal-to-distal axis of the electrode, and at least one protrusion which tilts (e.g. is angled) toward the central proximal-to-distal axis of the electrode. In an example, a set of electroconductive proximal protrusions can comprise at least three protrusions, including: at least one protrusion which extends out from the distal portion at an obtuse angle; at least one protrusion which extends out from the distal portion at a right angle; and at least one protrusion which extends out from the distal portion at an acute angle.

In an example, a set of electroconductive proximal protrusions can comprise at least three protrusions, including: at least one protrusion which extends out from the distal portion at a 135 degree angle; at least one protrusion which extends out from the distal portion at a 90 degree angle; and at least one protrusion which extends out from the distal portion at a 45 degree angle. In an example, a set of electroconductive proximal protrusions can comprise at least three protrusions, including: at least one protrusion which extends out from the distal portion at a 120 degree angle; at least one protrusion which extends out from the distal portion at a 90 degree angle; and at least one protrusion which extends out from the distal portion at a 60 degree angle.

In an example, a set of electroconductive proximal protrusions can comprise at least three protrusions which extend out different polar angles relative to a central point, wherein the polar angles are evenly distributed around a 360-degree circle. In an example, a set of electroconductive proximal protrusions can comprise three protrusions which extend out at different polar angles relative to a central point and wherein the polar angles of neighboring protrusions differ by 120 degrees. In an example, a set of electroconductive proximal protrusions can comprise four protrusions which extend out at different polar angles relative to a central point and wherein the polar angles of neighboring protrusions differ by 90 degrees.

In an example, a set of electroconductive proximal protrusions can comprise six protrusions which extend out at different polar angles relative to a central point and wherein the polar angles of neighboring protrusions differ by 60 degrees. In an example, a set of electroconductive proximal protrusions can comprise eight protrusions which extend out at different polar angles relative to a central point and wherein the polar angles of neighboring protrusions differ by 45 degrees.

In an example, a set of electroconductive proximal protrusions, wherein neighboring pairs of protrusions have longitudinal axial vectors which differ from each other by 120 degrees. In an example, a set of electroconductive proximal protrusions, wherein neighboring pairs of protrusions have longitudinal axial vectors which differ from each other by 90 degrees. In an example, a set of electroconductive proximal protrusions, wherein neighboring pairs of protrusions have longitudinal axial vectors which differ from each other by 60 degrees. In an example, a set of electroconductive proximal protrusions, wherein neighboring pairs of protrusions have longitudinal axial vectors which differ from each other by 45 degrees. In an example, sets of proximal protrusions from an EEG electrode look (technically speaking) “like a bunch of chicken feet.” Example variations discussed elsewhere in this disclosure or priority-linked disclosures can be applied to this example where relevant.

FIG. 137 shows a cross-sectional view of an EEG electrode for use on a hair-covered portion of a person's head comprising: a distal portion 13701; and a plurality of sets of electroconductive proximal protrusions (e.g. pins, prongs, teeth, spikes, fingers, and/or protrusions) which extend out from the distal portion toward the person's head in order to penetrate between hairs and come into contact with the surface of the person's head; wherein (with the possible exception of a set of electroconductive proximal protrusions at the center of the electrode) a set of electroconductive proximal protrusions further comprises: at least one proximal protrusion 13702 whose proximal end tilts (e.g. is angled) away from a central proximal-to-distal axis of the electrode; and at least one proximal protrusion 13703 whose proximal end tilts (e.g. is angled) toward the central proximal-to-distal axis of the electrode.

In an example, an EEG can have a plurality of sets of protrusions which extend outward toward the surface of a person's head. In an example, two protrusions in a set have longitudinal axes whose (shadow) projections onto the same virtual two-dimensional plane differ by 90 degrees. In an example, two protrusions in a set have longitudinal axes whose (shadow) projections onto the same virtual two-dimensional plane intersect at a 90-degree angle. In an example, two protrusions in a set have longitudinal axes whose (shadow) projections onto the same virtual two-dimensional plane differ by between 55 and 95 degrees. In an example, two protrusions in a set have longitudinal axes whose (shadow) projections onto the same virtual two-dimensional plane intersect at an angle between 55 and 95 degrees. Example variations discussed elsewhere in this disclosure or priority-linked disclosures can be applied to this example where relevant.

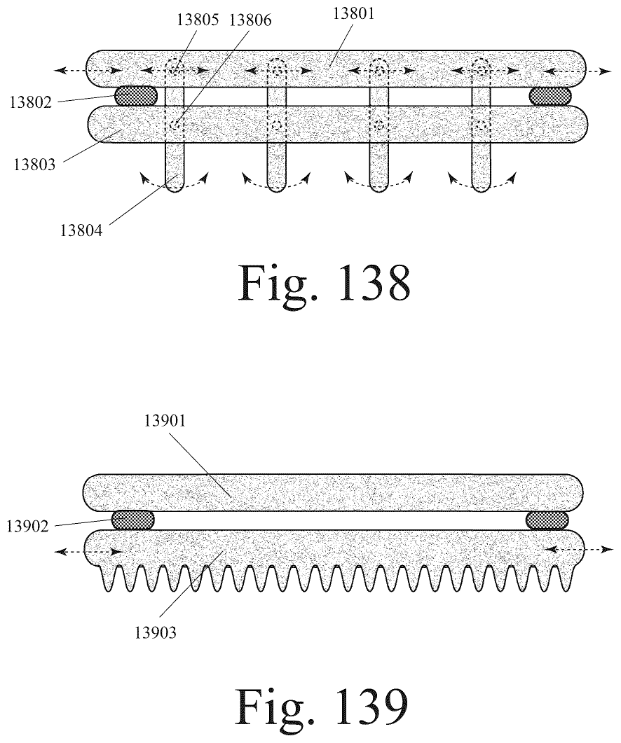

FIG. 138 shows a cross-sectional view of an EEG electrode for use on a hair-covered portion of a person's head comprising: a distal portion 13801; a proximal portion 13803; one or more actuators, including actuator 13802, which move (e.g. shift) the distal portion relative to the proximal portion, or vice versa; and a plurality of electroconductive proximal protrusions (e.g. pins, prongs, teeth, spikes, fingers, and/or protrusions), including protrusion 13804, which extend out toward the person's head in order to penetrate between hairs and come into contact with the surface of the person's head, wherein the proximal protrusions are moveably connected to the distal portion and to the proximal portion, and wherein movement of the distal portion relative to the proximal portion, or vice versa, causes the proximal protrusions to move (e.g. shift) relative to the surface of the person's head, thereby enhancing the ability of the protrusions to penetrate between hairs. In this example, the proximal protrusions are moveably connected to the distal and proximal portions by joints (e.g. joints or axles), including joints 13805 and 13806. In an example, an actuator can be an electromagnetic motor.

In an example, distal and proximal portions can be substantially parallel to each other. In an example, distal and proximal portions can be substantially-parallel disks, squares, or rounded squares. In an example, movement of a distal portion of an electrode relative to a proximal portion of the electrode, or vice versa, can be lateral shifting (or sliding) of one portion relative to the other. In an example, movement of one a distal portion of the electrode relative to a proximal portion of the electrode, or vice versa, can be lateral or vertical vibration. In an example, lateral shifting (or sliding) of a distal portion relative to a proximal portion, or vice versa, can cause lateral movement of protrusions relative to the surface of a person's head, thereby enhancing the ability of the protrusions to penetrate between hairs and contact the surface of the person's head.

In an example, protrusions can contact the surface of a person's head in a perpendicular manner (e.g. at a perpendicular angle). In an example, the angles at which protrusions contact the surface of a person's head can vary as actuators move the distal portion relative to the proximal portion, or vice versa. In an example, one of the angles at which protrusions can contact the surface of the person's head is 90 degrees. In an example, one of the ways in which protrusions can contact the surface of the person's head in a perpendicular manner. In an example, protrusions are not restricted to contacting the surface of the person's head in a non-perpendicular manner.

In an example, angles at which protrusions contact a person's head can vary within a range of 45 to 135 degrees as actuators move the distal and/or proximal portions of the electrode. In an example, angles at which protrusions contact a person's head can vary within a range of 60 to 120 degrees as actuators move the distal and/or proximal portions of the electrode. In an example, angles at which protrusions contact a person's head can vary within a range of 75 to 105 degrees as actuators move the distal and/or proximal portions of the electrode.

In an example, protrusions can extend out from the main body of the electrode in a perpendicular manner (e.g. at a perpendicular angle). In an example, angles at which protrusions extend out proximally from the proximal portion of the electrode can vary as actuators move the distal portion relative to the proximal portion, or vice versa. In an example, one of the angles by which protrusions can extend out proximally from the proximal portion of the electrode is 90 degrees. In an example, one of the ways in which protrusions can extend out from the proximal portion of the electrode is perpendicularly. Protrusions are not restricted to extending out from the proximal portion only a non-perpendicular manner.

In an example, angles at which protrusions extend out from the proximal portion can vary within a range of 45 to 135 degrees as actuators move the distal and/or proximal portions of the electrode. In an example, angles at which protrusions extend out from the proximal portion can vary within a range of 60 to 120 degrees as actuators move the distal and/or proximal portions of the electrode. In an example, angles at which protrusions extend out from the proximal portion can vary within a range of 75 to 105 degrees as actuators move the distal and/or proximal portions of the electrode. Example variations discussed elsewhere in this disclosure or priority-linked disclosures can be applied to this example where relevant.

FIG. 139 shows a cross-sectional view of an EEG electrode for use on a hair-covered portion of a person's head comprising: a distal portion 13901; a proximal portion 13903, wherein the proximal side of the proximal portion has a sinusoidal shape which enables it to penetrate between hairs and come into contact with the surface of a person's head; and one or more actuators, including actuator 13902, which move (e.g. shift) the proximal portion relative to the distal portion. Distal means farther from the surface of a person's head and proximal means closer to the surface of the person's head. In an example, an actuator can be an electromagnetic motor.

In an example, distal and proximal portions can be substantially parallel to each other. In an example, distal and proximal portions can be substantially-parallel disks, squares, or rounded squares. In an example, movement of a proximal portion of an electrode can be lateral shifting or sliding. In an example, movement of a proximal portion of an electrode can be vibration. In an example, shifting, sliding, or vibration of a proximal portion can enable sinusoidal protrusions to penetrate between hairs and contact the surface of the person's head. In an example, sinusoidal protrusions can extend out from the main body of an electrode in a perpendicular manner.

In an example, sinusoidal protrusions on a proximal portion of an electrode can be sinusoidal ridges. In an example, a proximal side can comprise a series of sinusoidal ridges which span an electrode laterally. In an example, sinusoidal protrusions can be sinusoidal rings. In an example, a proximal side can comprise a series of nested sinusoidal rings around the center of an electrode. In an example, sinusoidal protrusions can be sinusoidal peaks. In an example, a proximal side of an electrode can have a two-directional sinusoidal surface (e.g. similar to interior of an egg carton). In an example, there can be between 2 and 8 sinusoidal ridges, rings, or peaks on an electrode. In an example, there can be between 5 and 15 sinusoidal ridges, rings, or peaks on an electrode. In an example, there can be between 10 and 30 sinusoidal ridges, rings, or peaks on an electrode.

In an example, sinusoidal protrusions on the proximal side of an electrode can all have the same amplitude. In an example, the amplitudes of sinusoidal protrusions on an electrode can vary with distance of protrusions from the center of the electrode. In an example, the amplitudes of sinusoidal protrusions on an electrode can increase with increased distance of the protrusions from the center of the electrode. In an example, the amplitudes of sinusoidal protrusions which are farther from the center of an electrode can be greater than the amplitudes of sinusoidal protrusions which are closer to the center of the electrode. In an example, the amplitudes of sinusoidal protrusions on an electrode can decrease with increased distance of the protrusions from the center of the electrode. In an example, the amplitudes of sinusoidal protrusions which are farther from the center of an electrode can be less than the amplitudes of sinusoidal protrusions which are closer to the center of the electrode.

In an example, sinusoidal protrusions on the proximal side of an electrode can all have the same wavelength. In an example, the wavelengths of sinusoidal protrusions on an electrode can vary with distance of protrusions from the center of the electrode. In an example, the wavelengths of sinusoidal protrusions on an electrode can increase with increased distance of the protrusions from the center of the electrode. In an example, the wavelengths of sinusoidal protrusions which are farther from the center of an electrode can be greater than the wavelengths of sinusoidal protrusions which are closer to the center of the electrode. In an example, the wavelengths of sinusoidal protrusions on an electrode can decrease with increased distance of the protrusions from the center of the electrode. In an example, the wavelengths of sinusoidal protrusions which are farther from the center of an electrode can be less than the wavelengths of sinusoidal protrusions which are closer to the center of the electrode.

In an example, sinusoidal ridges, rings, or peaks can be made from an inherently-nonconductive polymer (e.g. PDMS) which has been made conductive by doping, impregnation, and/or coating with carbon structures (e.g. carbon nanotubes). In an example, sinusoidal ridges, rings, or peaks can be made from an inherently-nonconductive polymer (e.g. PDMS) which has been made conductive by doping, impregnation, and/or coating with metal (e.g. silver). In an alternative example, a proximal side of an electrode can have sawtooth wave shape instead of a sinusoidal wave shape. Example variations discussed elsewhere in this disclosure or priority-linked disclosures can be applied to this example where relevant.

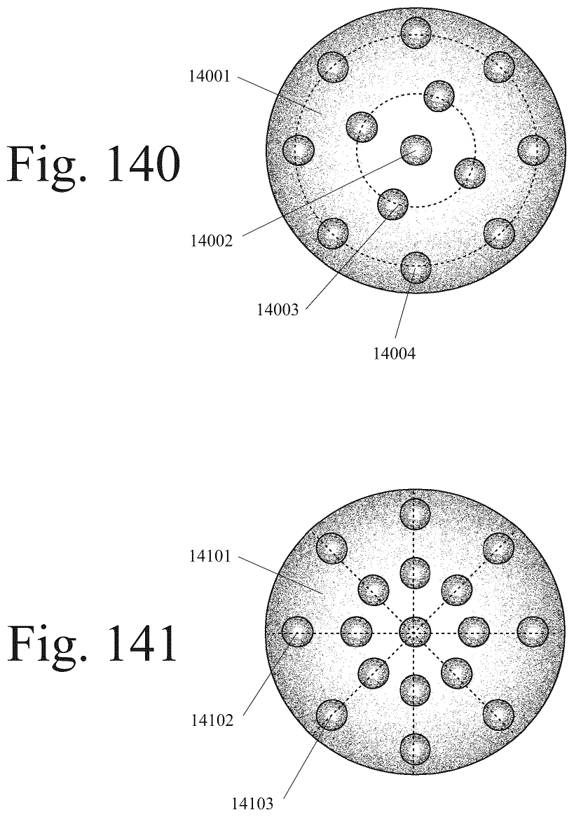

FIG. 140 shows a proximal side view of an EEG electrode for use on a hair-covered portion of a person's head, wherein the proximal side is the side which faces toward the surface of a person's head. This EEG electrode comprises: an electrode base 14001; and a plurality of electroconductive proximal protrusions (e.g. pins, prongs, teeth, spikes, fingers, and/or protrusions), including protrusions 14002, 14003, and 14004, which extend out from the electrode base toward the person's head in order to penetrate between hairs and come into contact with the surface of the person's head; wherein the plurality of electroconductive proximal protrusions further comprises at least two nested (e.g. concentric) rings of protrusions. In this example, protrusion 14003 is part of a first (inner) ring of protrusions and protrusion 14004 is part of a second (outer) ring of protrusions. In this example, the plurality of electroconductive proximal protrusions also includes a central protrusion 14002 which is not part of a ring.

In an example, an electrode base can have a circular and/or disk shape. In an example, an electrode base can have an elliptical, oval, and/or oblong shape. In an example, an electrode base can have a (rounded) square or (rounded) rectangular shape. In an example, a protrusion can have a columnar shape. In an example, a protrusion can have a conic, conic section, and/or frustal shape. In an example, a protrusion can have a parabolic and/or paraboloidal shape. In an example, a protrusion can have a hemispherical shape. In an example, a protrusion can extend out from an electrode base in a perpendicular manner.

In an example, an electrode can have two nested (e.g. concentric) rings of protrusions extending from a base toward the surface of a person's head. In an example, an electrode can have two nested (e.g. concentric) rings of protrusions extending from a base toward the surface of a person's head, plus a central protrusion extending from axial center of the base. In an example, an electrode can have three or more nested (e.g. concentric) rings of protrusions extending from a base toward the surface of a person's head. In an example, an electrode can have three or more nested (e.g. concentric) rings of protrusions extending from a base toward the surface of a person's head, plus a central protrusion extending from the center of the base.

In an example, the number of protrusions in a ring can be greater for outer rings (which are farther from the center of the electrode) than for inner rings (which are closer to the center of the electrode). In an example, an inner ring can have four protrusions and an outer ring can have six protrusions. In an example, an inner ring can have four protrusions and an outer ring can have eight protrusions. In an example, an inner ring can have six protrusions and an outer ring can have nine protrusions. In an example, an inner ring can have six protrusions and an outer ring can have twelve protrusions.

In an example, protrusions in an outer ring (farther from the center of the electrode) can be longer than protrusions in an inner ring (closer to the center of the electrode). In an example, protrusions in an outer ring (farther from the center of the electrode) can be more flexible, more compressible, and/or have a lower durometer than protrusions in an inner ring (closer to the center of the electrode). In an example, protrusions in an outer ring (farther from the center of the electrode) can be tilted and/or angled toward the center of the electrode more than protrusions in an inner ring (closer to the center of the electrode).

In an example, protrusions in an outer ring (farther from the center of the electrode) can be tilted and/or angled away from center of the electrode more than protrusions in an inner ring (closer to the center of the electrode). In an example, protrusions in an outer ring (farther from the center of the electrode) can be shorter than protrusions in an inner ring (closer to the center of the electrode). In an example, protrusions in an outer ring (farther from the center of the electrode) can be less flexible, less compressible, and/or have a higher durometer than protrusions in an inner ring (closer to the center of the electrode). Example variations discussed elsewhere in this disclosure or priority-linked disclosures can be applied to this example where relevant.

FIG. 141 shows a proximal side view of an EEG electrode for use on a hair-covered portion of a person's head, wherein the proximal side is the side which faces toward the surface of a person's head. This EEG electrode comprises: an electrode base 14101; and a plurality of electroconductive proximal protrusions (e.g. pins, prongs, teeth, spikes, fingers, and/or protrusions), including protrusions 14102 and 14103, which extend out from the electrode base toward the person's head in order to penetrate between hairs and come into contact with the surface of the person's head; wherein the plurality of electroconductive proximal protrusions has a radial spoke (e.g. hub and spoke) configuration. In this example, protrusion 14102 is part of a first radial line (e.g. spoke) of protrusions and protrusion 14103 is part of a second radial line (e.g. spoke) of protrusions.

In this example, an electrode base has a circular and/or disk shape. In an example, an electrode base can have an elliptical, oval, and/or oblong shape. In an example, an electrode base can have a (rounded) square or (rounded) rectangular shape. In an example, a protrusion can have a columnar shape. In an example, a protrusion can have a conic, conic section, and/or frustal shape. In an example, a protrusion can have a parabolic and/or paraboloidal shape. In an example, a protrusion can have a hemispherical shape. In an example, a protrusion can extend out from an electrode base in a perpendicular manner.

In this example, an electrode has eight radial lines, series, or spokes of protrusions. These radial lines extend out from the center of the electrode base. Alternatively, an electrode can have six radial lines, series, or spokes of protrusions. In an example, protrusions which are farther from the center of the base can be longer than protrusions which are closer to the center of the base. In an example, protrusions which are farther from the center of the base can be more flexible, more compressible, and/or have a lower durometer than protrusions which are closer to the center of the base. In an example, protrusions which are farther from the center of the base can be tilted and/or angled toward the center of the base more than protrusions which are closer to the center of the base.

In an example, protrusions which are farther from the center of the base can be shorter than protrusions which are closer to the center of the base. In an example, protrusions which are farther from the center of the base can be less flexible, less compressible, and/or have a higher durometer than protrusions which are closer to the center of the base. In an example, protrusions which are farther from the center of the base can be tilted and/or angled away from the center of the base more than protrusions which are closer to the center of the base. Example variations discussed elsewhere in this disclosure or priority-linked disclosures can be applied to this example where relevant.

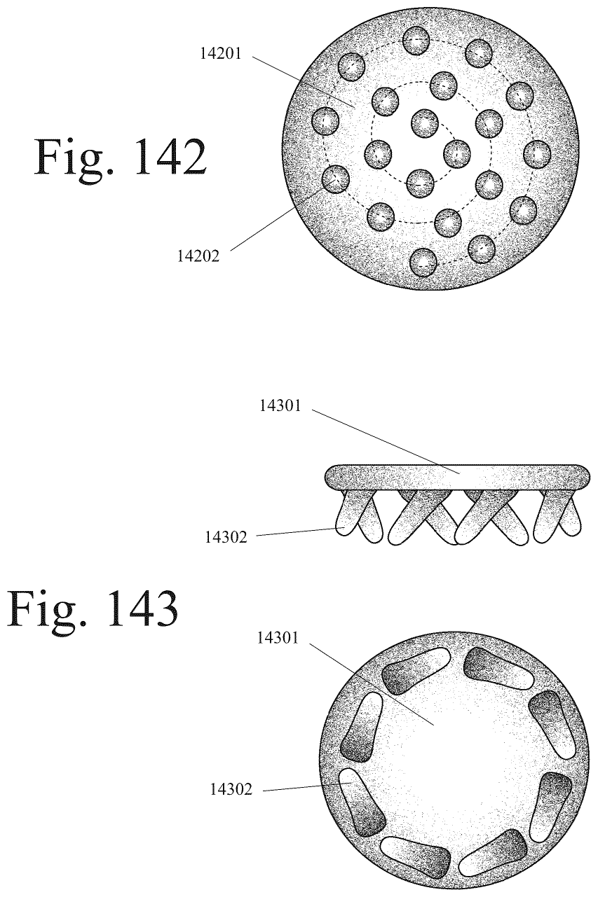

FIG. 142 shows a proximal side view of an EEG electrode for use on a hair-covered portion of a person's head, wherein the proximal side is the side which faces toward the surface of a person's head. This EEG electrode comprises: an electrode base 14201; and a plurality of electroconductive proximal protrusions (e.g. pins, prongs, teeth, spikes, fingers, and/or protrusions), including protrusion 14202, which extend out from the electrode base toward the person's head in order to penetrate between hairs and come into contact with the surface of the person's head; wherein the plurality of electroconductive proximal protrusions has a spiral configuration. Example variations discussed elsewhere in this disclosure or priority-linked disclosures can be applied to this example where relevant.

The upper portion of FIG. 143 shows a proximal side view and the lower portion of FIG. 143 shows a lateral side view of an EEG electrode for use on a hair-covered portion of a person's head, wherein the proximal side is the side which faces toward the surface of a person's head. This EEG electrode comprises: an electrode base 14301; and a ring of electroconductive proximal protrusions (e.g. pins, prongs, teeth, spikes, fingers, and/or protrusions), including protrusion 14302, which extend out in a non-perpendicular manner from the electrode base toward the person's head in order to penetrate between hairs and come into contact with the surface of the person's head.

In this example, the proximal end of each protrusion is tilted toward the neighboring protrusion, forming a “head-to-toe” ring of protrusions in the proximal side view. In this example, all of the protrusions extend out from the electrode base at the same, non-perpendicular angle, but they extend in different directions. In this example, all of the protrusions in the ring extend out from the electrode base at the same, non-perpendicular angle, but the directions in which they extend vary and are substantially-aligned with the circle of the ring. Example variations discussed elsewhere in this disclosure or priority-linked disclosures can be applied to this example where relevant.

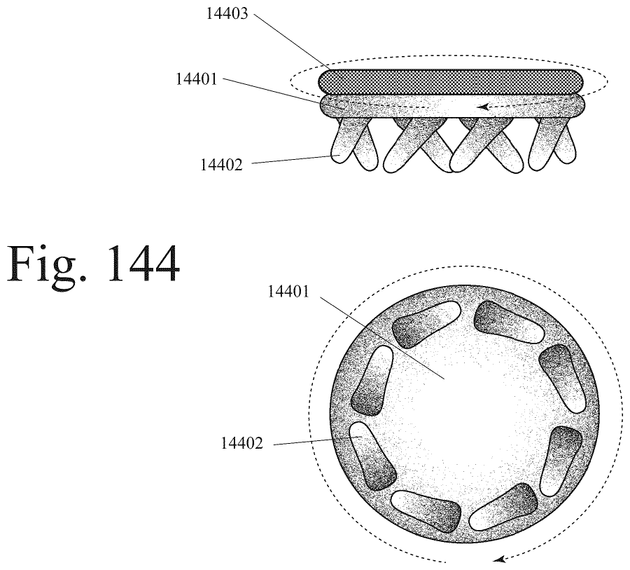

The upper portion of FIG. 144 shows a proximal side view and the lower portion of FIG. 144 shows a lateral side view of an EEG electrode for use on a hair-covered portion of a person's head, wherein the proximal side is the side which faces toward the surface of a person's head. This EEG electrode comprises: an electrode base 14401; a ring of electroconductive proximal protrusions (e.g. pins, prongs, teeth, spikes, fingers, and/or protrusions), including protrusion 14402, which extend out in a non-perpendicular manner from the electrode base toward the person's head in order to penetrate between hairs and come into contact with the surface of the person's head; and an actuator 14403 which rotates the electrode base.

In this example, the proximal end of each protrusion is tilted toward the neighboring protrusion, forming a “head-to-toe” ring of protrusions in the proximal side view. In this example, all of the protrusions extend out from the electrode base at the same, non-perpendicular angle, but they extend in different directions. In this example, all of the protrusions in the ring extend out from the electrode base at the same, non-perpendicular angle, but the directions in which they extend vary and are substantially-aligned with the circle of the ring. Example variations discussed elsewhere in this disclosure or priority-linked disclosures can be applied to this example where relevant.

The upper portion of FIG. 144 shows a proximal side view and the lower portion of FIG. 144 shows a lateral side view of an EEG electrode for use on a hair-covered portion of a person's head, wherein the proximal side is the side which faces toward the surface of a person's head. This EEG electrode comprises: an electrode base 14401; a ring of electroconductive proximal protrusions (e.g. pins, prongs, teeth, spikes, fingers, and/or protrusions), including protrusion 14402, which extend out in a non-perpendicular manner from the electrode base toward the person's head in order to penetrate between hairs and come into contact with the surface of the person's head; and an actuator 14403 which rotates the electrode base. Example variations discussed elsewhere in this disclosure or priority-linked disclosures can be applied to this example where relevant.

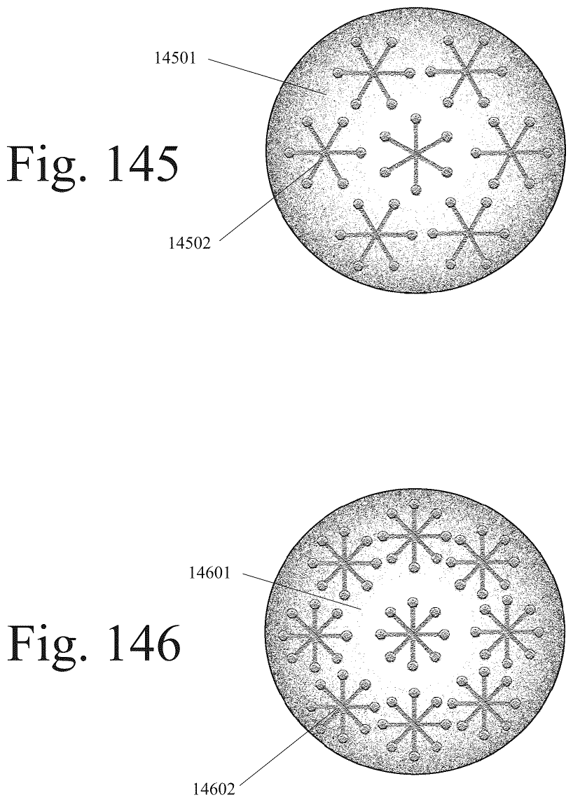

FIG. 145 shows a proximal side view of an EEG electrode for use on a hair-covered portion of a person's head, wherein the proximal side is the side which faces toward the surface of a person's head. This EEG electrode comprises: an electrode base 14501; a plurality of sets of electroconductive proximal protrusions (e.g. pins, prongs, teeth, spikes, fingers, and/or protrusions), including set 14502, which extend out in a non-perpendicular manner from the electrode base toward the person's head in order to penetrate between hairs and come into contact with the surface of the person's head; wherein each set further comprises six protrusions which extend out radially from the center of the set.

In an example, from a proximal side perspective, a set of six protrusions can look like an asterisk. In an example, a set of protrusions can comprise six equidistant spokes. In an example, a set of protrusions can comprise six spokes which are evenly distributed around the circumference of the set. In an example, the ends of the six protrusions can form the vertexes of a (virtual) equilateral hexagon. In an example, from a proximal side perspective, each protrusion in a set can intersect its neighboring protrusion at a 60-degree angle. In an example, each pair of neighboring protrusions (e.g. a pair of protrusions which are closest together) in a set can be separated by the same distance. In an example, a protrusion can be generally columnar, with a ball at its end. Example variations discussed elsewhere in this disclosure or priority-linked disclosures can be applied to this example where relevant.

FIG. 146 shows a proximal side view of an EEG electrode for use on a hair-covered portion of a person's head, wherein the proximal side is the side which faces toward the surface of a person's head. This EEG electrode comprises: an electrode base 14601; a plurality of sets of electroconductive proximal protrusions (e.g. pins, prongs, teeth, spikes, fingers, and/or protrusions), including set 14602, which extend out in a non-perpendicular manner from the electrode base toward the person's head in order to penetrate between hairs and come into contact with the surface of the person's head; wherein each set further comprises eight protrusions which extend out radially from the center of the set.

In an example, a set of protrusions can comprise eight equidistant spokes. In an example, a set of protrusions can comprise eight spokes which are evenly distributed around the circumference of the set. In an example, the ends of the eight protrusions can form the vertexes of a (virtual) equilateral octagon. In an example, from a proximal side perspective, each protrusion in a set can intersect its neighboring protrusion at a 45-degree angle. In an example, each pair of neighboring protrusions (e.g. a pair of protrusions which are closest together) in a set can be separated by the same distance. In an example, a protrusion can be generally columnar, with a ball at its end. Example variations discussed elsewhere in this disclosure or priority-linked disclosures can be applied to this example where relevant.

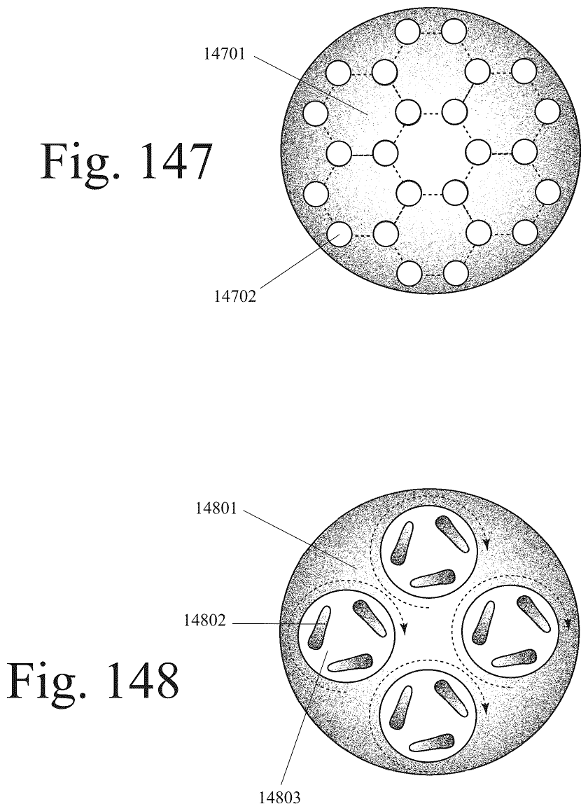

FIG. 147 shows a proximal side view of an EEG electrode for use on a hair-covered portion of a person's head, wherein the proximal side is the side which faces toward the surface of a person's head. This EEG electrode comprises: an electrode base 14701; a hexagonal grid (e.g. honeycomb grid) of electroconductive proximal protrusions (e.g. pins, prongs, teeth, spikes, fingers, and/or protrusions), including set 14702, which extend out in a non-perpendicular manner from the electrode base toward the person's head in order to penetrate between hairs and come into contact with the surface of the person's head, wherein there is a protrusion at each vertex of a hexagon in the hexagonal grid. Example variations discussed elsewhere in this disclosure or priority-linked disclosures can be applied to this example where relevant.

FIG. 148 shows a proximal side view of an EEG electrode for use on a hair-covered portion of a person's head, wherein the proximal side is the side which faces toward the surface of a person's head. This EEG electrode comprises: a distal electrode base 14801; at least two rotatable sets (including set 14803) of electroconductive proximal protrusions (including protrusion 14802) which extend out from the electrode base toward the person's head in order to penetrate between hairs and come into contact with the surface of the person's head.

In an example, a set of protrusions can be a ring of protrusions. In an example, protrusions in a revolving set of protrusions can extend outward from an electrode base in a perpendicular manner (e.g. at 90-degree angles relative to the electrode base). In an example, protrusions in a revolving set of protrusions can extend outward from an electrode base in a non-perpendicular manner (e.g. at acute or obtuse angles relative to the electrode base). In an example, protrusions in a revolving set of protrusions can all extend outward from an electrode base at the same angle relative to the electrode base. In an example, protrusions in a set can be tilted or angled in the same direction that a set is rotated so that the tips of the protrusions tend to slide between stands of hair when the set is rotated.

In an example, an electrode can comprise at least two sets of proximal protrusions, wherein each set can be individually rotated (e.g. rotated and/or revolved) to enhance the ability of the protrusions to penetrate between hairs and come into contact with the surface of a person's head. In an example, an electrode can further comprise one or more actuators which automatically rotate one or more sets of protrusions. In an example, there can be at least three protrusions in each set. In an example, protrusions can extend outward from the electrode base in a non-perpendicular manner. In an example, protrusions in a set can all extend outward from the electrode base at the same angle, although in different directions.

In an example, an EEG electrode for use on a hair-covered portion of a person's head can comprise: a distal electrode base; at least two rotatable sets of electroconductive proximal protrusions which extend out from the electrode base toward the person's head in order to penetrate between hairs and come into contact with the surface of the person's head, wherein rotation of a set of protrusions enhances the ability of the protrusions to penetrate between hairs and come into contact with the surface of a person's head. In an example, the EEG electrode can further comprise one or more actuators (e.g. electromagnetic motors) which automatically rotate the sets of protrusions. Example variations discussed elsewhere in this disclosure or priority-linked disclosures can be applied to this example where relevant.

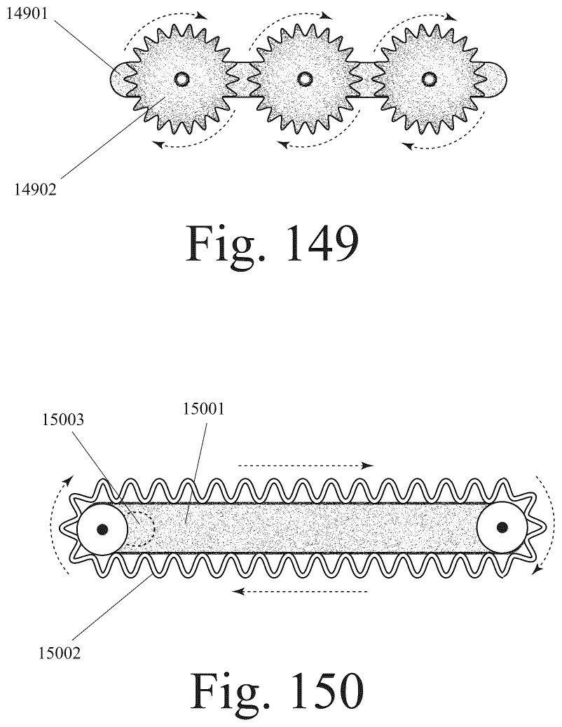

FIG. 149 shows a side cross-sectional view of an EEG electrode for use on a hair-covered portion of a person's head comprising: an electrode base 14901; and a plurality of rotatable wheels (e.g. wheels, disks, or gears), including 14902; wherein a rotatable wheel has protrusions (e.g. protrusions, teeth, or cogs) around the wheel's perimeter which penetrate between hairs and come into contact with the surface of the person's head. In an example, an electrode can further comprise axles connected to the electrode base around which the wheels rotate. In an example, rotation of the wheels enhances the ability of protrusions on the wheels to penetrate between hairs and come into contact with the surface of a person's head.

In an example, an electrode base can be generally parallel to the surface of a person's head and rotatable wheels can be generally perpendicular to the surface of the person's head. In an example, an electrode can comprise at least three rotatable wheels with protrusions around their perimeters. In an example, an electrode can comprise six rotatable wheels with protrusions around their perimeters. In an example, protrusions on wheels can be rounded. In an example, the perimeter of a wheel can have a sinusoidal shape (e.g. a shape formed by applying a sine wave to a circle). In an example, protrusions on wheels can have vertexes. In an example, the perimeter of a wheel can have a sawtooth shape (e.g. a shape formed by applying a sawtooth wave to a circle).

In an example, rotatable wheels can be passively rotated by rolling contact with the person's head. In an example, the electrode can further comprise one or more actuators (e.g. electromagnetic motors) which actively rotate the one or more wheels. In an example, an actuator can rotate a wheel back and forth, in an iterative and/or oscillating manner, in clockwise and counterclockwise directions. In an example, an actuator can “wiggle” or vibrate a wheel back and forth.

In an example, protrusions on a wheel can be made with one or more materials selected from the group consisting of: metal; inherently-conductive polymer; inherently-nonconductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal. Example variations discussed elsewhere in this disclosure or priority-linked disclosures can be applied to this example where relevant.

FIG. 150 shows a side cross-sectional view of an EEG electrode for use on a hair-covered portion of a person's head comprising: an electrode base 15001; and a rotating loop 15002 of protrusions which penetrate between hairs and come into contact with the surface of the person's head; and one or more actuators (e.g. electromagnetic motors), such as 15003, which rotate the loop.

In an example, an electrode can comprise a loop of protrusions which rotates around an electrode base. In an example, an electrode can comprise a loop of protrusions which rotates around the perimeter of an electrode base. In an example, an electrode can comprise an oblong or circular loop of protrusions which rotates around an electrode base. In an example, an electrode can comprise a rotating chain-link loop of protrusions. In an example, an electrode can comprise a rotating trend, rotating track, and/or rotating chain-link loop of protrusions which penetrate between hairs and come into contact with the surface of the person's head. In an example, an electrode can comprise a rotating sinusoidal loop of protrusions. Example variations discussed elsewhere in this disclosure or priority-linked disclosures can be applied to this example where relevant.

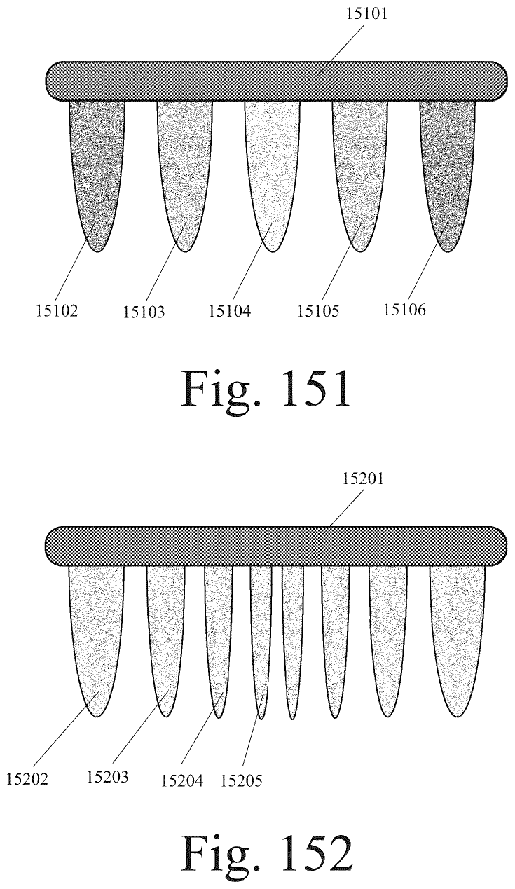

FIG. 151 shows a side cross-sectional view of an EEG electrode for use on a hair-covered portion of a person's head comprising: a distal electrode base 15101; and a plurality of electroconductive proximal protrusions (e.g. pins, prongs, teeth, spikes, fingers, and/or protrusions), including protrusions 15102, 15103, 15104, 15105, and 15106, which extend out from the distal electrode base toward the person's head in order to penetrate between hairs and come into contact with the surface of the person's head; wherein a first protrusion which is a first distance from the center of the electrode base is made from a first material, wherein a second protrusion which is a second distance from the center of the electrode base is made from a second material, and wherein the second distance is greater than the first distance. In this example, protrusions 15103 and 15105 are made from a first material and protrusions 15102 and 15106 are made from a second material. In this example, protrusion 15104 is made from a third material.

In an example, the second material can be more elastic than the first material. In an example, the second material can be more flexible than the first material. In an example, the second material can be softer than the first material. In an example, the second material can be more compressible than the first material. In an example, the second material can have a lower durometer level than the first material. In an example, the second material can have a lower Young's modulus than the first material. In an example, the second material can be less electroconductive than the first material. In an example, the second material can be made with a lower percentage of metal than the first material.

In an example, the first material can be more elastic than the second material. In an example, the first material can be more flexible than the second material. In an example, the first material can be softer than the second material. In an example, the first material can be more compressible than the second material. In an example, the first material can have a lower durometer level than the second material. In an example, the first material can have a lower Young's modulus than the second material. In an example, the first material can be less electroconductive than the second material. In an example, the first material can be made with a lower percentage of metal than the second material.

FIG. 151 also shows an EEG electrode for use on a hair-covered portion of a person's head can comprise: a distal electrode base 15101; and a plurality of electroconductive proximal protrusions (e.g. pins, prongs, teeth, spikes, fingers, and/or protrusions) which extend out from the distal electrode base toward the person's head in order to penetrate between hairs and come into contact with the surface of the person's head; wherein a first protrusion 15104 which is a first distance from the center of the electrode base is made from a first material, wherein a second protrusion 15103 or 15105 which is a second distance from the center of the electrode base is made from a second material, wherein a third protrusion 15102 or 15106 which is a third distance from the center of the electrode base is made from a third material, wherein the second distance is greater than the first distance, and wherein the third distance is greater than the second distance.

In an example, the second material can be more elastic than the first material and the third material can be more elastic than the second material. In an example, the second material can be more flexible than the first material and the third material can be more flexible than the second material. In an example, the second material can be softer than the first material and the third material can be softer than the second material. In an example, the second material can be more compressible than the first material and the third material can be more compressible than the second material.

In an example, the second material can have a lower durometer level than the first material and the third material can have a lower durometer level than the second material. In an example, the second material can have a lower Young's modulus than the first material and the third material can have a lower Young's modulus than the second material. In an example, the second material can be less electroconductive than the first material and the third material can be less electroconductive than the second material. In an example, the second material can be made with a lower percentage of metal than the first material and the third material can be made with a lower percentage of metal than the second material.

In an example, the second material can be more elastic than the third material and the first material can be more elastic than the second material. In an example, the second material can be more flexible than the third material and the first material can be more flexible than the second material. In an example, the second material can be softer than the third material and the first material can be softer than the second material. In an example, the second material can be more compressible than the third material and the first material can be more compressible than the second material.

In an example, the second material can have a lower durometer level than the third material and the first material can have a lower durometer level than the second material. In an example, the second material can have a lower Young's modulus than the third material and the first material can have a lower Young's modulus than the second material. In an example, the second material can be less electroconductive than the third material and the first material can be less electroconductive than the second material. In an example, the second material can be made with a lower percentage of metal than the third material and the first material can be made with a lower percentage of metal than the second material.

In an example, a plurality of protrusions can comprise nested (e.g. concentric) rings of protrusions. In an example, an inner ring of protrusions can be made with a first material and an outer ring of protrusions can be made with a second material. In an example, the materials from which protrusions are made can have one or more attributes which differ with distance from the center of an electrode base. In an example, one of more protrusion material attributes which vary with distance from the center of an electrode base can be selected from the group consisting of: elasticity, flexibility, softness, compressibility, durometer level, Young's modulus, electroconductivity, and percentage of metal. In an example, one of more protrusion material attributes which vary with distance from the center of an electrode base can be selected from the group consisting of: hardness, stiffness, impedance, and percentage of non-conductive polymer.

In an example, a protrusion can have a parabolic and/or paraboloidal shape. In an example, a protrusion can have a sinusoidal shape. In an example, a protrusion can have a columnar shape. In an example, a protrusion can have a conic or conic-section shape. In an example, a protrusion can have a frustal shape. In an example, an electrode base can have a circular or disk shape. In an example, an electrode base can have an elliptical, oblong, or oval shape. In an example, an electrode base can have a (rounded) square or (rounded) rectangular shape. Example variations discussed elsewhere in this disclosure or priority-linked disclosures can be applied to this example where relevant.

FIG. 152 shows a side cross-sectional view of an EEG electrode for use on a hair-covered portion of a person's head comprising: a distal electrode base 15201; and a plurality of electroconductive proximal protrusions (e.g. pins, prongs, teeth, spikes, fingers, and/or protrusions), including protrusions 15202, 15203, 15204, and 15205, which extend out from the distal electrode base toward the person's head in order to penetrate between hairs and come into contact with the surface of the person's head; wherein a first protrusion 15205 which is a first distance from the center of the electrode base has a first width, wherein a second protrusion 15204 which is a second distance from the center of the electrode base has a second width, and wherein the second width is different than the first width. In this example, the second width is greater than the first width. In an alternative example, the second width can be less than the first width.

FIG. 152 also shows a side cross-sectional view of an EEG electrode for use on a hair-covered portion of a person's head comprising: a distal electrode base 15201; and a plurality of electroconductive proximal protrusions (e.g. pins, prongs, teeth, spikes, fingers, and/or protrusions), including protrusions 15202, 15203, 15204, and 15205, which extend out from the distal electrode base toward the person's head in order to penetrate between hairs and come into contact with the surface of the person's head; wherein a first protrusion 15205 which is a first distance from the center of the electrode base has a first width, wherein a second protrusion 15204 which is a second distance from the center of the electrode base has a second width, wherein a third protrusion 15203 which is a third distance from the center of the electrode base has a third width, wherein the second width is different than the first width, and wherein the third width is different than the second width. In this example, the second width is greater than the first width and the third width is greater than the second width. In an alternative example, the second width can be less than the first width and the third width can be less than the second width.

In an example, a plurality of protrusions can comprise nested (e.g. concentric) rings of protrusions. In an example, a protrusion can have a parabolic and/or paraboloidal shape. In an example, a protrusion can have a sinusoidal shape. In an example, a protrusion can have a columnar shape. In an example, a protrusion can have a conic or conic-section shape. In an example, a protrusion can have a frustal shape. In an example, an electrode base can have a circular or disk shape. In an example, an electrode base can have an elliptical, oblong, or oval shape. In an example, an electrode base can have a (rounded) square or (rounded) rectangular shape. Example variations discussed elsewhere in this disclosure or priority-linked disclosures can be applied to this example where relevant.

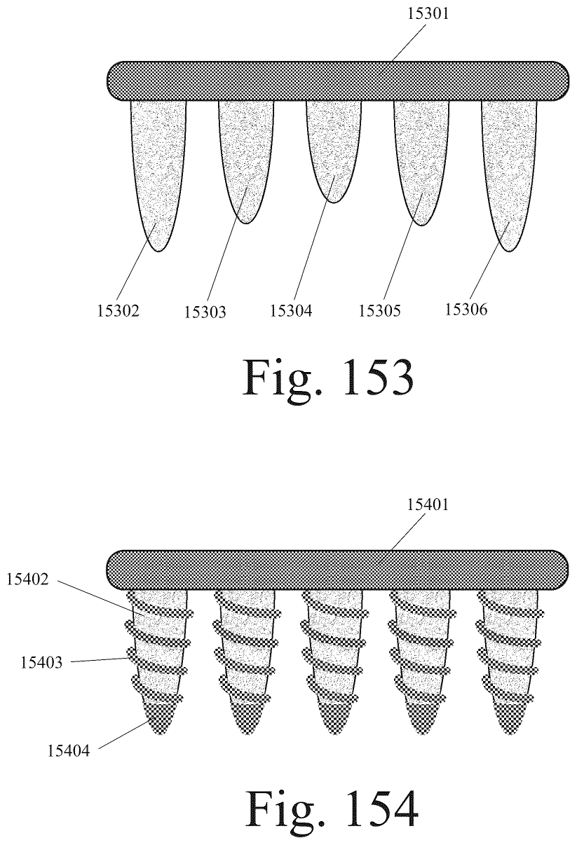

FIG. 153 shows a side cross-sectional view of an EEG electrode for use on a hair-covered portion of a person's head comprising: a distal electrode base 15301; and a plurality of electroconductive proximal protrusions (e.g. pins, prongs, teeth, spikes, fingers, and/or protrusions), including protrusions 15302, 15303, 15304, 15305, and 15306, which extend out from the distal electrode base toward the person's head in order to penetrate between hairs and come into contact with the surface of the person's head; wherein a first protrusion which is a first distance from the center of the electrode base has a first length, wherein a second protrusion which is a second distance from the center of the electrode base has a second length, and wherein the second length is different than the first length. In this example, the second length is greater than the first length. In an alternative example, the second length can be less than the first length.

FIG. 153 also shows a side cross-sectional view of an EEG electrode for use on a hair-covered portion of a person's head comprising: a distal electrode base 15301; and a plurality of electroconductive proximal protrusions (e.g. pins, prongs, teeth, spikes, fingers, and/or protrusions), including protrusions 15302, 15303, 15304, 15305, and 15306, which extend out from the distal electrode base toward the person's head in order to penetrate between hairs and come into contact with the surface of the person's head; wherein a first protrusion 15304 which is a first distance from the center of the electrode base has a first length, wherein a second protrusion 15303 or 15305 which is a second distance from the center of the electrode base has a second length, wherein a third protrusion 15302 or 15306 which is a third distance from the center of the electrode base has a third length, wherein the second length is different than the first length, and wherein the third length is different than the second length. In this example, the second length is greater than the first length and the third length is greater than the second length. In an alternative example, the second length can be less than the first length and the third length can be less than the second length.

In an example, a plurality of protrusions can comprise nested (e.g. concentric) rings of protrusions. In an example, a protrusion can have a parabolic and/or paraboloidal shape. In an example, a protrusion can have a sinusoidal shape. In an example, a protrusion can have a columnar shape. In an example, a protrusion can have a conic or conic-section shape. In an example, a protrusion can have a frustal shape. In an example, an electrode base can have a circular or disk shape. In an example, an electrode base can have an elliptical, oblong, or oval shape. In an example, an electrode base can have a (rounded) square or (rounded) rectangular shape. Example variations discussed elsewhere in this disclosure or priority-linked disclosures can be applied to this example where relevant.

FIG. 154 shows a side cross-sectional view of an EEG electrode for use on a hair-covered portion of a person's head comprising: a distal electrode base 15401; and a plurality of proximal protrusions (e.g. pins, prongs, teeth, spikes, fingers, and/or protrusions), including protrusion 15402, which extend out from the distal electrode base toward the person's head in order to penetrate between hairs and come into contact with the surface of the person's head; wherein a protrusion is encircled by a helical conductive spring (or coil) 15403; and wherein the tip of a protrusion has a conductive cap 15404 which is attached to the helical conductive spring (or coil). In an example a helical conductive spring (or coil) can be made from a conductive metal. In an example, a helical conductive spring (or coil) can be made from a conductive polymer.

In an example, a protrusion can have a parabolic and/or paraboloidal shape. In an example, a protrusion can have a sinusoidal shape. In an example, a protrusion can have a columnar shape. In an example, a protrusion can have a conic or conic-section shape. In an example, a protrusion can have a frustal shape. In an example, an electrode base can have a circular or disk shape. In an example, an electrode base can have an elliptical, oblong, or oval shape. In an example, an electrode base can have a (rounded) square or (rounded) rectangular shape. Example variations discussed elsewhere in this disclosure or priority-linked disclosures can be applied to this example where relevant.

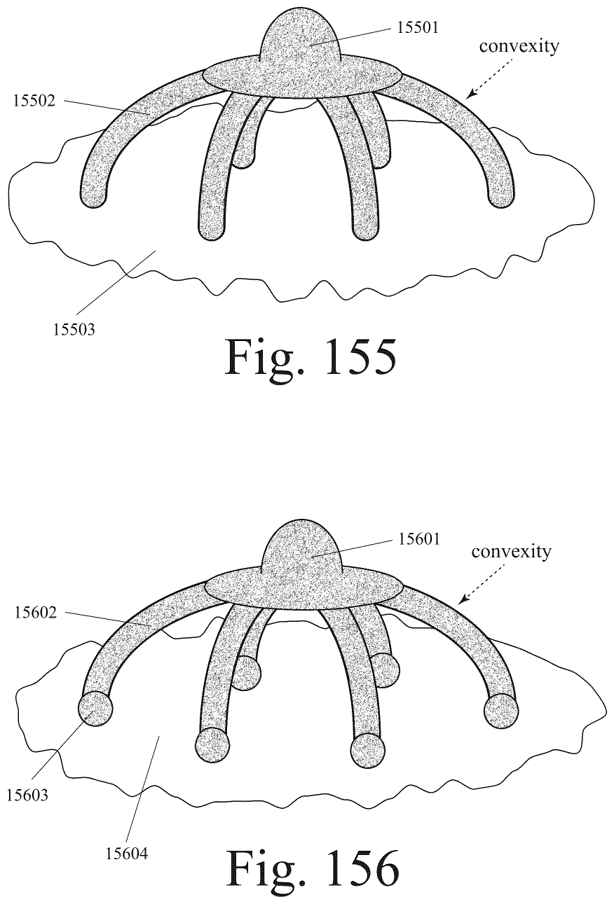

FIG. 155 shows a side cross-sectional view of an EEG electrode for use on a hair-covered portion of a person's head comprising: an electrode hub 15501; and a plurality of arcuate conductive legs (e.g. legs, arms, fingers, branches, spokes, or protrusions), including leg 15502, which extend out radially from the electrode hub and also extend out toward the surface of a person's head 15503 in order to penetrate between hairs and come into contact with the surface of the person's head; wherein a middle portion of a leg bows away from the surface of the person's head. In an example, a conductive leg can be convex, wherein a middle portion of the leg bows away from the surface of a person's head.

In an example, an electrode with a hub and a plurality of radially-extending legs can be symmetric with respect to a central proximal-to-distal plane. In an example, radially-extending legs can be evenly distributed around the circumference of an electrode hub. In an example, an electrode can comprise a distal hub and six conductive legs which extend out radially from the hub and toward the surface of a person's head. In an example, six radially-extending legs can be pair-wise separated by 60-degree angles. In an example, neighboring legs (or virtual extensions thereof) can intersect at 60-degree angles. In an example, the proximal ends of six legs can form the vertexes of a virtual equilateral hexagon.

In an example, an electrode can comprise a distal hub and eight conductive legs which extend out radially from the hub and toward the surface of a person's head. In an example, eight radially-extending legs can be pair-wise separated by 45-degree angles. In an example, neighboring legs (or virtual extensions thereof) can intersect at 45-degree angles. In an example, the proximal ends of eight legs can form the vertexes of a virtual equilateral octagon. In an example, an electrode can comprise a distal hub and twelve conductive legs which extend out radially from the hub and toward the surface of a person's head. In an example, twelve radially-extending legs can be pair-wise separated by 30-degree angles. In an example, neighboring legs (or virtual extensions thereof), can intersect at 30-degree angles). In an example, the proximal ends of twelve legs can form the vertexes of a virtual equilateral dodecagon.

In an example, an arcuate conductive leg can be a single, continuous piece. In an example, an arcuate conductive leg can be a single, continuous piece which is made from a metal. In an example, an arcuate conductive leg can be a single, continuous piece which is made from a conductive polymer material. In an example, an arcuate conductive leg can be a single, continuous piece which is made from a polymer (e.g. PDMS) which has been doped, impregnated, and/or coated with carbon structures (e.g. carbon nanotubes) or a metal. In an example, an arcuate conductive leg can be flexible. In an example, an arcuate conductive leg can be articulated. In an example, an arcuate conductive leg can comprise two articulated and moveably-connected segments.

In an example, an arcuate conductive leg can have a shape which is a conic section. In an example, an arcuate conductive leg can have a shape which is a section (e.g. a quarter or half) of a circle or ellipse. In an example, a plurality of arcuate legs can comprise arcs of a section of a sphere or ellipsoid. In an example, the proximal end of an arcuate conductive leg can intersect the surface of a person's head in a perpendicular manner (e.g. at a 90-degree angle). In an example, the proximal end of an arcuate conductive leg can intersect the surface of a person's head at an acute angle. In an example, the proximal end of an arcuate conductive leg can be rounded. Example variations discussed elsewhere in this disclosure or priority-linked disclosures can be applied to this example where relevant.

FIG. 156 shows a side cross-sectional view of an EEG electrode for use on a hair-covered portion of a person's head comprising: an electrode hub 15601; and a plurality of arcuate conductive legs (e.g. legs, arms, fingers, branches, spokes, or protrusions), including leg 15602, which extend out radially from the electrode hub and also extend out toward the surface of a person's head 15604 in order to penetrate between hairs and come into contact with the surface of the person's head; wherein a middle portion of a leg bows away from the surface of the person's head; and wherein there is a conductive ball 15603 on the proximal end of each leg. In an example, a conductive ball can be a separate piece which is attached to a leg. Alternatively, a conductive ball can be an integral portion of a continuous single-piece leg.

In an example, a conductive leg can be convex, wherein a middle portion of the leg bows away from the surface of a person's head. In an example, an electrode with a hub and a plurality of radially-extending legs can be symmetric with respect to a central proximal-to-distal plane. In an example, radially-extending legs can be evenly distributed around the circumference of an electrode hub. In an example, an electrode can comprise a distal hub and six conductive legs which extend out radially from the hub and toward the surface of a person's head. In an example, six radially-extending legs can be pair-wise separated by 60-degree angles. In an example, neighboring legs (or virtual extensions thereof) can intersect at 60-degree angles. In an example, the proximal ends of six legs can form the vertexes of a virtual equilateral hexagon.

In an example, an electrode can comprise a distal hub and eight conductive legs which extend out radially from the hub and toward the surface of a person's head. In an example, eight radially-extending legs can be pair-wise separated by 45-degree angles. In an example, neighboring legs (or virtual extensions thereof) can intersect at 45-degree angles. In an example, the proximal ends of eight legs can form the vertexes of a virtual equilateral octagon. In an example, an electrode with eight legs can be formed in an arthro podcast. In an example, an electrode can comprise a distal hub and twelve conductive legs which extend out radially from the hub and toward the surface of a person's head. In an example, twelve radially-extending legs can be pair-wise separated by 30-degree angles. In an example, neighboring legs (or virtual extensions thereof), can intersect at 30-degree angles). In an example, the proximal ends of twelve legs can form the vertexes of a virtual equilateral dodecagon.

In an example, an arcuate conductive leg can be a single, continuous piece. In an example, an arcuate conductive leg can be a single, continuous piece which is made from a metal. In an example, an arcuate conductive leg can be a single, continuous piece which is made from a conductive polymer material. In an example, an arcuate conductive leg can be a single, continuous piece which is made from a polymer (e.g. PDMS) which has been doped, impregnated, and/or coated with carbon structures (e.g. carbon nanotubes) or a metal. In an example, an arcuate conductive leg can be flexible. In an example, an arcuate conductive leg can be articulated. In an example, an arcuate conductive leg can comprise two articulated and moveably-connected segments.

In an example, an arcuate conductive leg can have a shape which is a conic section. In an example, an arcuate conductive leg can have a shape which is a section (e.g. a quarter or half) of a circle or ellipse. In an example, a plurality of arcuate legs can comprise arcs of a section of a sphere or ellipsoid. In an example, the proximal end of an arcuate conductive leg can intersect the surface of a person's head in a perpendicular manner (e.g. at a 90-degree angle). In an example, the proximal end of an arcuate conductive leg can intersect the surface of a person's head at an acute angle. Example variations discussed elsewhere in this disclosure or priority-linked disclosures can be applied to this example where relevant.

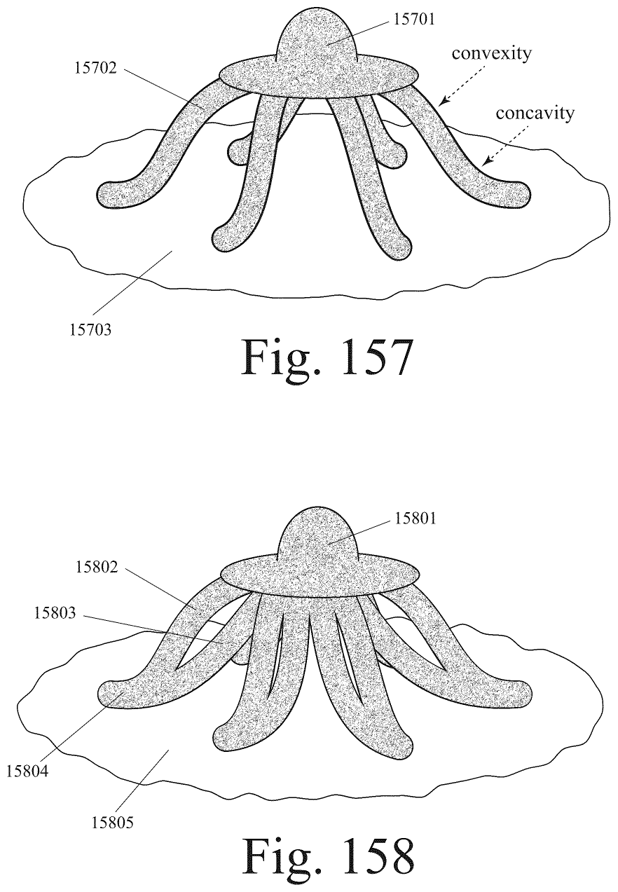

FIG. 157 shows a side cross-sectional view of an EEG electrode for use on a hair-covered portion of a person's head comprising: an electrode hub 15701; and a plurality of arcuate conductive legs (e.g. legs, arms, fingers, branches, spokes, or protrusions), including leg 15702, which extend out radially from the electrode hub and also extend out toward the surface of a person's head 15703 in order to penetrate between hairs and come into contact with the surface of the person's head; wherein a leg has a convex portion which bows away from the surface of the person's head and a concave portion which bows toward the surface of the person's head.

In an example, a convex portion of a leg can be closer to an electrode hub than a concave portion of the leg. In an example, the half of a leg which is closer to an electrode hub can bow (e.g. bow, bend, or curve) away from the surface of a person's head and the half of the leg which is farther from the hub can bow (e.g. bow, bend, or curve) toward the surface of the person's head. In another example, a convex portion of a leg can be farther from an electrode hub than a concave portion of the leg. In another example, the half of a leg which is farther from an electrode hub can bow (e.g. bow, bend, or curve) away from the surface of a person's head and the half of the leg which is closer to the hub can bow (e.g. bow, bend, or curve) toward the surface of the person's head.

In an example, an electrode with a hub and a plurality of radially-extending legs can be symmetric with respect to a central proximal-to-distal plane. In an example, radially-extending legs can be evenly distributed around the circumference of an electrode hub. In an example, an electrode can comprise a distal hub and six conductive legs which extend out radially from the hub and toward the surface of a person's head. In an example, six radially-extending legs can be pair-wise separated by 60-degree angles. In an example, neighboring legs (or virtual extensions thereof) can intersect at 60-degree angles. In an example, the proximal ends of six legs can form the vertexes of a virtual equilateral hexagon.

In an example, an electrode can comprise a distal hub and eight conductive legs which extend out radially from the hub and toward the surface of a person's head. In an example, eight radially-extending legs can be pair-wise separated by 45-degree angles. In an example, neighboring legs (or virtual extensions thereof) can intersect at 45-degree angles. In an example, the proximal ends of eight legs can form the vertexes of a virtual equilateral octagon. In an example, an electrode with eight legs can be formed in an arthro podcast. In an example, an electrode can comprise a distal hub and twelve conductive legs which extend out radially from the hub and toward the surface of a person's head. In an example, twelve radially-extending legs can be pair-wise separated by 30-degree angles. In an example, neighboring legs (or virtual extensions thereof), can intersect at 30-degree angles). In an example, the proximal ends of twelve legs can form the vertexes of a virtual equilateral dodecagon.

In an example, an arcuate conductive leg can be a single, continuous piece. In an example, an arcuate conductive leg can be a single, continuous piece which is made from a metal. In an example, an arcuate conductive leg can be a single, continuous piece which is made from a conductive polymer material. In an example, an arcuate conductive leg can be a single, continuous piece which is made from a polymer (e.g. PDMS) which has been doped, impregnated, and/or coated with carbon structures (e.g. carbon nanotubes) or a metal. In an example, an arcuate conductive leg can be flexible. In an example, an arcuate conductive leg can be articulated. In an example, an arcuate conductive leg can comprise two articulated and moveably-connected segments.

In an example, an arcuate conductive leg can have a shape which is a conic section. In an example, an arcuate conductive leg can have a shape which is a section (e.g. a quarter or half) of a circle or ellipse. In an example, a plurality of arcuate legs can comprise arcs of a section of a sphere or ellipsoid. Example variations discussed elsewhere in this disclosure or priority-linked disclosures can be applied to this example where relevant.

FIG. 158 shows a side cross-sectional view of an EEG electrode for use on a hair-covered portion of a person's head comprising: an electrode hub 15801; and a plurality of bifurcated conductive legs (e.g. legs, arms, fingers, branches, spokes, or protrusions), including leg 15802, which extend out radially from the electrode hub and also extend out toward the surface of a person's head 15803 in order to penetrate between hairs and come into contact with the surface of the person's head; wherein the middle portion of a leg is bifurcated, but distal and proximal ends of the leg are not bifurcated.

In an example, in the middle portion of a leg where the leg bifurcates, one branch can be convex and the other branch can be concave. In an example, an electrode with a hub and a plurality of radially-extending legs can be symmetric with respect to a central proximal-to-distal plane. In an example, radially-extending legs can be evenly distributed around the circumference of an electrode hub. In an example, an arcuate conductive leg can be flexible. Example variations discussed elsewhere in this disclosure or priority-linked disclosures can be applied to this example where relevant.

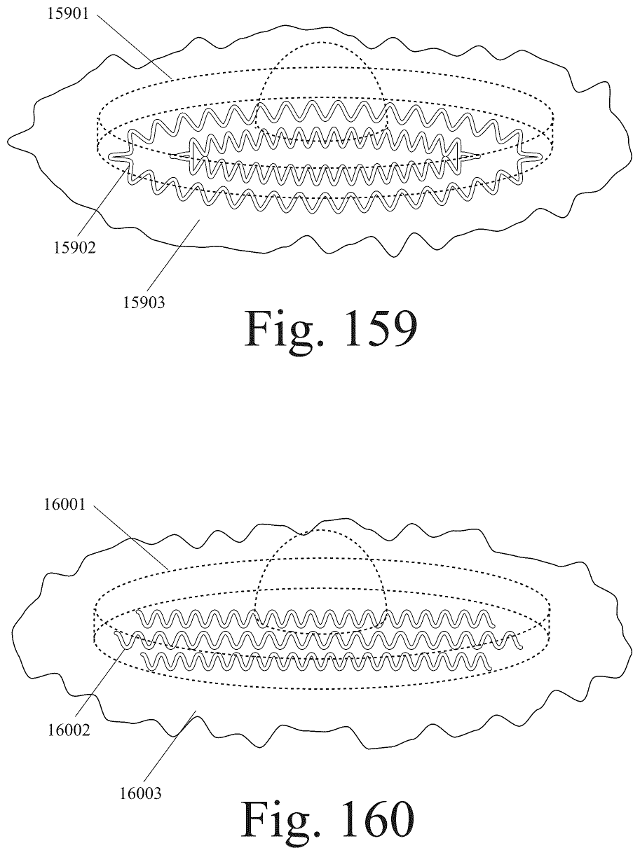

FIG. 159 shows a side cross-sectional view of an EEG electrode for use on a hair-covered portion of a person's head comprising: a distal electrode base 15901; and a plurality of nested (e.g. concentric) sinusoidal conductive rings, including 15902, on the proximal side of the base which penetrate between hairs and come into contact with the surface of the person's head 15903.

In an example, the amplitude of a sinusoidal curve of an outer nested sinusoidal ring can be different than the amplitude of a sinusoidal curve of an inner nested sinusoidal ring. In an example, the amplitude of a sinusoidal curve of an outer nested sinusoidal ring can be greater than the amplitude of a sinusoidal curve of an inner nested sinusoidal ring. In an example, the wavelength of the sinusoidal curve of an outer nested sinusoidal ring can be different than the wavelength of the sinusoidal curve of an inner nested sinusoidal ring. In an example, the wavelength of the sinusoidal curve of an outer nested sinusoidal ring can be greater than the wavelength of the sinusoidal curve of an inner nested sinusoidal ring.

In another example, the amplitude of the sinusoidal curve of an outer nested sinusoidal ring can be less than the amplitude of the sinusoidal curve of an inner nested sinusoidal ring. In another example, the wavelength of the sinusoidal curve of an outer nested sinusoidal ring can be less than the wavelength of the sinusoidal curve of an inner nested sinusoidal ring. In another example, rings can have sawtooth-shaped protrusions instead of sinusoidal protrusions. Example variations discussed elsewhere in this disclosure or priority-linked disclosures can be applied to this example where relevant.

FIG. 160 shows a side cross-sectional view of an EEG electrode for use on a hair-covered portion of a person's head comprising: a distal electrode base 16001; and a plurality of parallel sinusoidal conductive ridges, including 16002, on the proximal side of the base which penetrate between hairs and come into contact with the surface of the person's head 16003. In another example, ridges can have a sawtooth shape instead of a sinusoidal shape. Example variations discussed elsewhere in this disclosure or priority-linked disclosures can be applied to this example where relevant.

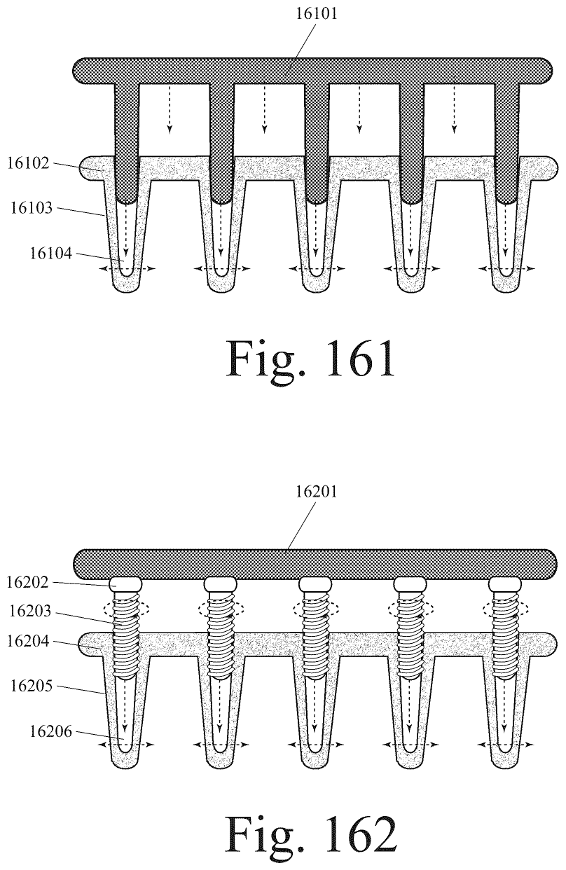

FIG. 161 shows a side cross-sectional view of an EEG electrode for use on a hair-covered portion of a person's head comprising: a first (distal) electrode portion 16101 which further comprises a plurality of first proximal protrusions extending out from the first electrode portion toward the surface of a person's head, and wherein a first proximal protrusion is made from a material with a first durometer level; and a second (proximal) electrode portion 16102 which further comprises a plurality of second protrusions (including protrusion 16103) extending out from the second electrode portion toward the surface of the person's head, wherein a second proximal protrusion is made from a material with a second durometer level, wherein the second durometer level is less than the first durometer level, wherein the second protrusion has a hollow core 16104 into which the first protrusion can be inserted, and wherein inserting the first protrusion into the hollow core of the second protrusion increases the stiffness, width, and/or length of the second protrusion, thereby enhancing the ability of the second protrusion to penetrate between hairs and contact the surface of a person's head. Example variations discussed elsewhere in this disclosure or priority-linked disclosures can be applied to this example where relevant.

FIG. 162 shows a side cross-sectional view of an EEG electrode for use on a hair-covered portion of a person's head comprising: a first (distal) electrode portion 16201; a second (proximal) electrode portion 16204 which further comprises a plurality of protrusions, including 16205, extending out from the second electrode portion toward the surface of the person's head; wherein a protrusion in the plurality of protrusions has a threaded hollow core 16206; a plurality of threaded cylinders, including 16203; and a plurality of actuators (e.g. electromagnetic motors), including 16202, which an actuator rotates a threaded cylinder; wherein rotational insertion of a threaded cylinder into a threaded hollow core of a protrusion increases the stiffness, width, and/or length of the protrusion, thereby enhancing the ability of the protrusion to penetrate between hairs and contact the surface of a person's head. Example variations discussed elsewhere in this disclosure or priority-linked disclosures can be applied to this example where relevant.

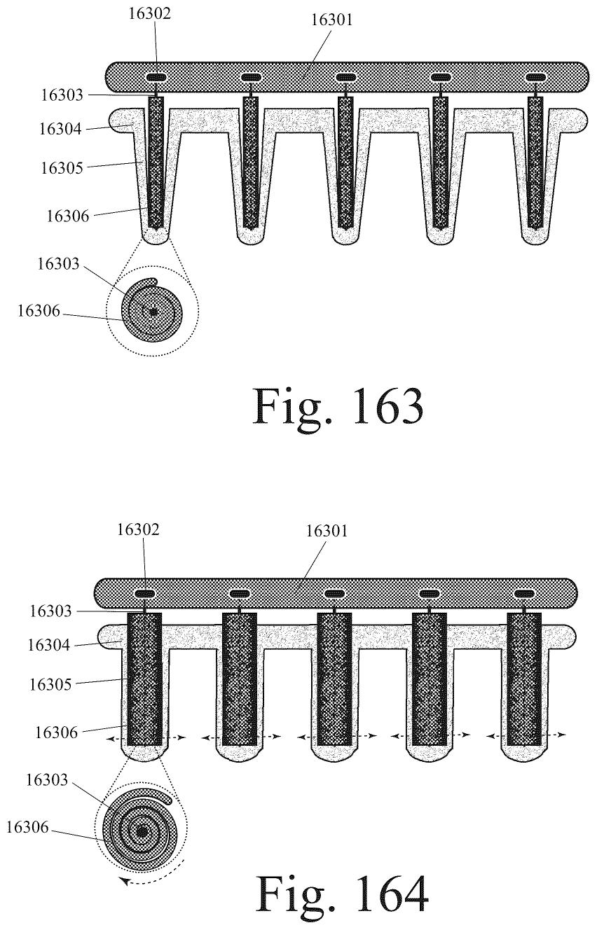

FIGS. 163 and 164 show two side cross-sectional views, at two different times, of an EEG electrode for use on a hair-covered portion of a person's head comprising: a distal electrode portion 16301; a proximal electrode portion 16304 which further comprises a plurality of protrusions, including 16305, extending out from the proximal electrode portion toward the surface of the person's head; wherein protrusions in the plurality of protrusions have hollow cores; a plurality of cylindrical coils, including 16306, which are coiled (e.g. rolled in a spiral manner) around central axles, including 16303, inside the hollow cores of the protrusions; and a plurality of actuators (e.g. electromagnetic motors), including 16302, which rotate the axles; wherein rotation of an axle in a first (e.g. clockwise) direction coils a cylindrical coil, which decreases the diameter of the cylindrical coil, which decreases the width (and/or stiffness) of the protrusion; and wherein rotation of an axle in a second (e.g. counter-clockwise) direction uncoils a cylindrical coil, which increases the diameter of the cylindrical coil, which increases the width (and/or stiffness) of the protrusion.

FIG. 163 shows this electrode at a first time wherein the cylindrical coils are tightly wound around their axles and the ends of the protrusions are relatively narrow and/or soft. FIG. 164 shows this electrode at a second time wherein the cylindrical coils are less rightly wound around their axles and the ends of the protrusions are relatively wide and/or stiff. The upper portions of FIGS. 163 and 164 shows side cross-sectional views of the entire electrode. The lower portions of FIGS. 163 and 164 display a dotted line circle which shows a close-up proximal cross-section of a protrusion. Example variations discussed elsewhere in this disclosure or priority-linked disclosures can be applied to this example where relevant.

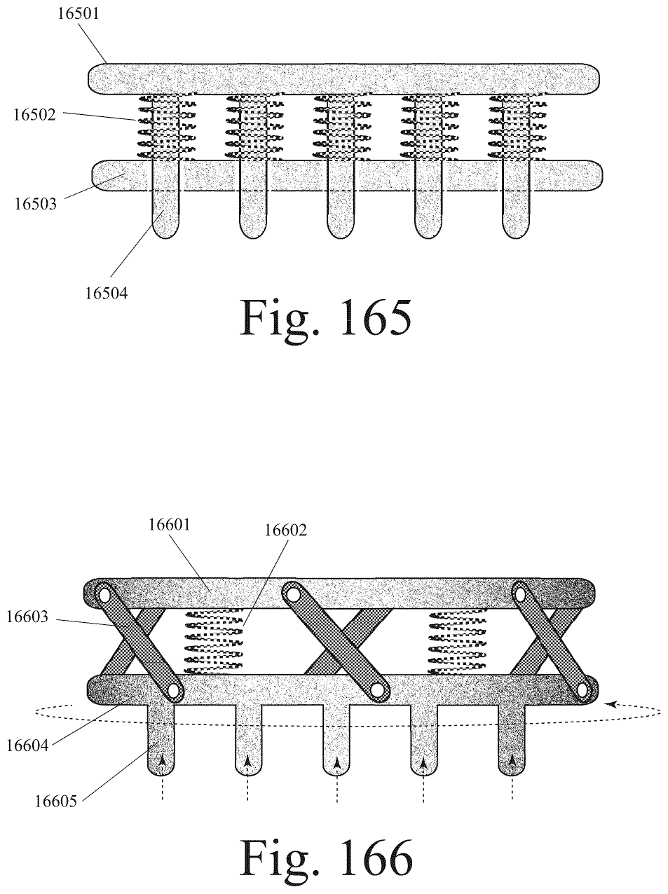

FIG. 165 shows a side cross-sectional view of an EEG electrode for use on a hair-covered portion of a person's head comprising: a distal electrode portion 16501; a proximal electrode portion 16503; a plurality of electroconductive protrusions, including 16504, which extend toward the surface of the person's head; and a plurality of springs (or coils), including 16502.

In an example, a plurality of springs (or coils) can be configured between a distal electrode portion and a proximal electrode portion. In an example, springs can compel a proximal electrode portion toward the surface of a person's head. In an example, springs can compel protrusions toward the surface of a person's head. In an example, there can be a spring connected to each protrusion. In an example, protrusions can extend through openings in a proximal electrode portion. In an example, protrusions can be integral parts of a proximal electrode portion. Example variations discussed elsewhere in this disclosure or priority-linked disclosures can be applied to this example where relevant.

FIG. 166 shows a side cross-sectional view of an EEG electrode for use on a hair-covered portion of a person's head comprising: a distal electrode portion 16601; a proximal electrode portion 16604 which further comprises a plurality of electroconductive protrusions, including 16605, which extend toward the surface of the person's head; a plurality of articulated movable struts, including 16603, which connect the distal electrode portion to the proximal electrode portion; and one or more springs (or coils), including 16602, between the distal electrode portion and the proximal electrode portion; wherein pressure from the surface of a person's head on the protrusions pushes the proximal electrode portion toward the distal electrode portion, which rotates the proximal electrode portion relative to the distal electrode portion (e.g. via angular movement and/or articulation of the movable struts), which slides the protrusions laterally relative to the surface of the person's head, which enables the protrusions to slide and penetrate between hairs on the surface of the person's head. Example variations discussed elsewhere in this disclosure or priority-linked disclosures can be applied to this example where relevant.

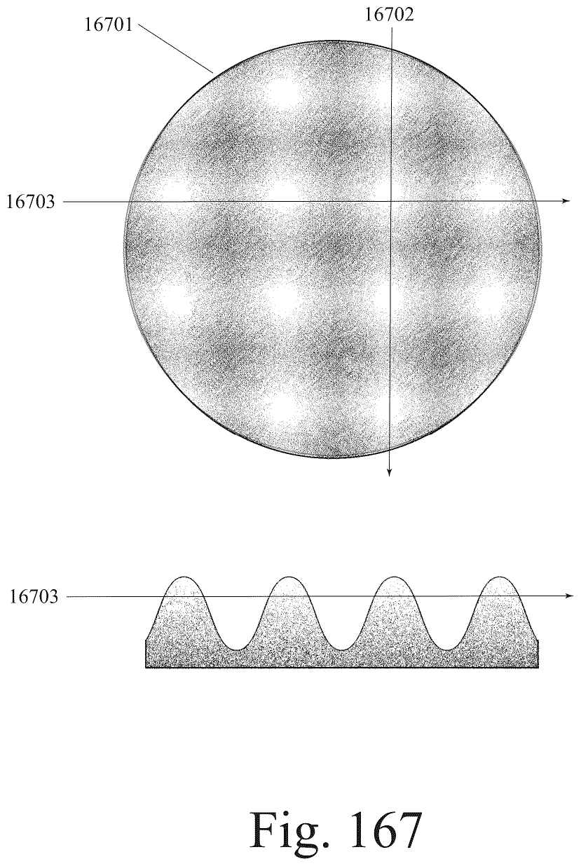

The upper portion of FIG. 167 shows a proximal view and the lower portion of FIG. 167 shows a side cross-sectional view of an EEG electrode 16701 for use on a hair-covered portion of a person's head comprising an orthogonal grid of sinusoidal electroconductive protrusions, wherein an orthogonal grid of sinusoidal electroconductive protrusions has a sinusoidal cross-sectional shape along each of two orthogonal axes, 16702 and 16703. In an alternative example, protrusions can have a sawtooth shape instead of a sinusoidal shape. Example variations discussed elsewhere in this disclosure or priority-linked disclosures can be applied to this example where relevant.

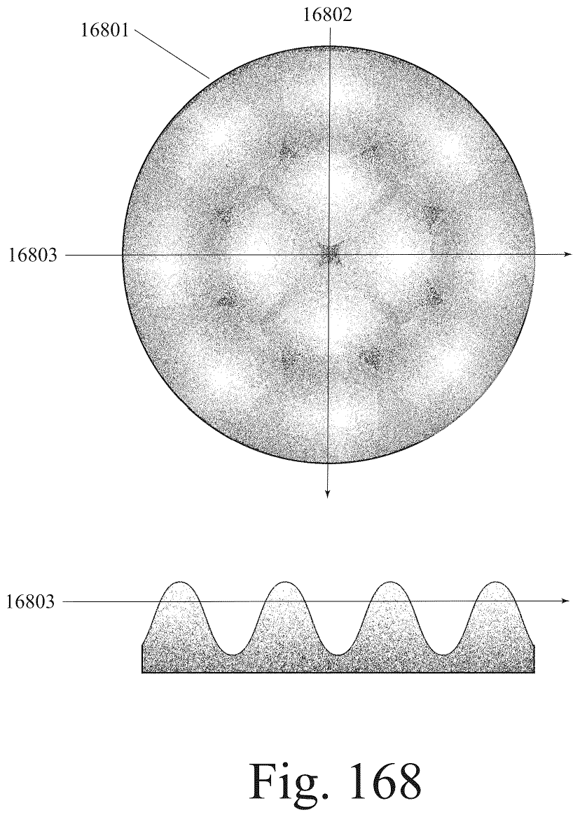

The upper portion of FIG. 168 shows a proximal view and the lower portion of FIG. 168 shows a side cross-sectional view of an EEG electrode 16801 for use on a hair-covered portion of a person's head comprising nested (e.g. concentric) rings of sinusoidal electroconductive protrusions. In this example, an electrode has a sinusoidal cross-sectional shape along each of two orthogonal axes, 16802 and 16803. In an alternative example, protrusions can have a sawtooth shape instead of a sinusoidal shape. Example variations discussed elsewhere in this disclosure or priority-linked disclosures can be applied to this example where relevant.

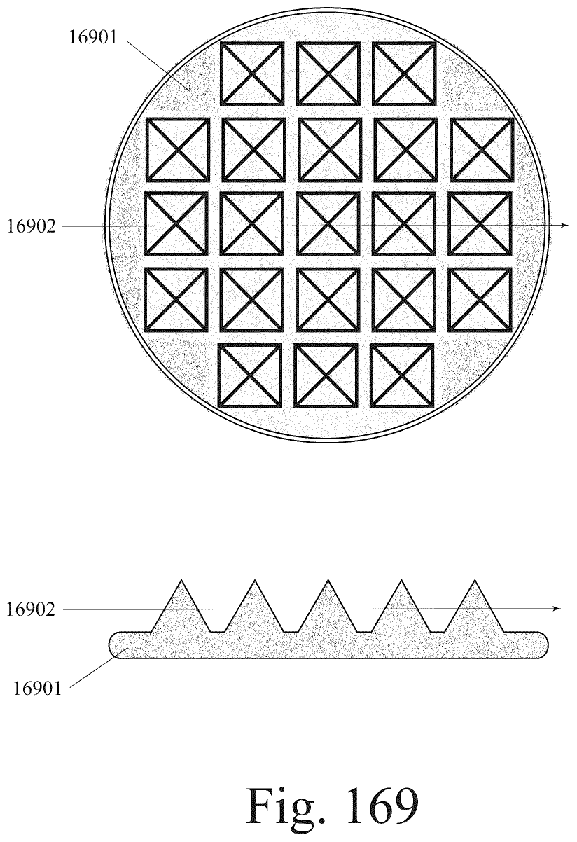

The upper portion of FIG. 169 shows a proximal view and the lower portion of FIG. 169 shows a side cross-sectional view of an EEG electrode for use on a hair-covered portion of a person's head comprising: a distal electrode base 16901; and a plurality of pyramid-shaped electroconductive protrusions which extend out from the electrode base toward the surface of a person's head. In this example, the plurality of pyramid-shaped electroconductive protrusions comprises an orthogonal (e.g. row-and-column) grid of protrusions, including row 16902. In another example, a plurality of pyramid-shaped electroconductive protrusions can comprise nested (e.g. concentric) rings of protrusions. In an example, a pyramid-shaped electroconductive protrusion can have a square base and a single proximal vertex. Example variations discussed elsewhere in this disclosure or priority-linked disclosures can be applied to this example where relevant.

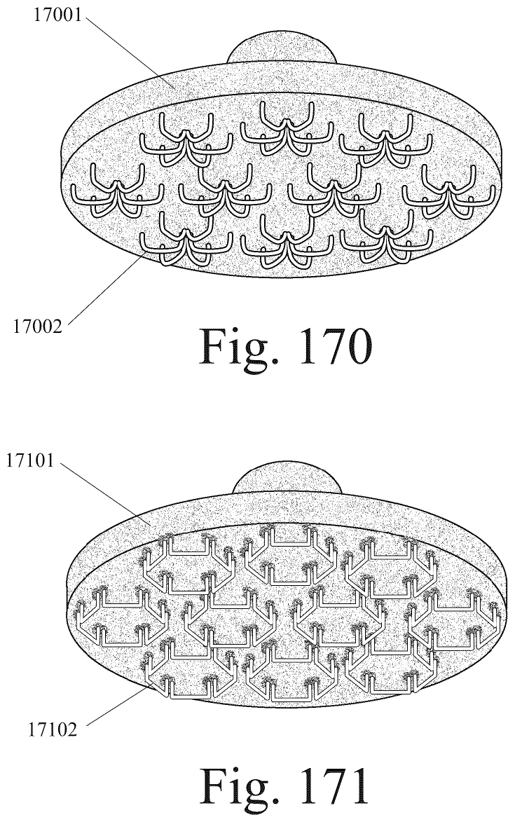

FIG. 170 shows an oblique proximal-to-side view of an EEG electrode for use on a hair-covered portion of a person's head comprising: an electrode base 17001; and a plurality of sets of electroconductive loops, including set 17002; wherein an electroconductive loop in a set of electroconductive loops extends out from the electrode base toward the surface of a person's head; wherein an electroconductive loop in a set of electroconductive loops is connected to the electrode base at two or more locations, including a central first location which is a first distance from the center of a set and a peripheral second location which is a second distance from the center of the set, and wherein the second distance is greater than the first distance.

In an example, electroconductive loops in a set of electroconductive loops can extend radially out from the center of a set as well as extend out from the electrode base toward the surface of a person's head. Differences in the radial orientations of different loops in a set enable different loops to penetrate between different stands of hair in different orientations relative to the electrode base. In an example, electroconductive loops can be evenly radially-distributed around the circumference of a set. In an example, pair-wise neighboring loops in a set can all be separated by (and/or virtually intersect at) the same inter-loop angle. In an example, polar coordinates of neighboring loops in a set which extend out radially from the center of the set can differ by the same number of degrees.

In an example, a set of electroconductive loops can include six radially-extending electroconductive loops in which neighboring loops are separated by (and/or virtually intersect at) 60-degree angles. In an example, peripheral second locations of loops in a set of loops can be located at the vertexes of a (virtual) equilateral hexagon. In an example, an EEG electrode for use on a hair-covered portion of a person's head can comprise: an electrode base; and a plurality of hexagonal sets of electroconductive loops, wherein there are six loops in a hexagonal set, and wherein loops in a hexagonal set extend out radially from the center of the set and also extend out from the electrode base toward the surface of a person's head.

In an example, a set of electroconductive loops can include eight radially-extending electroconductive loops in which neighboring loops are separated by (and/or virtually intersect at) 45-degree angles. In an example, the (peripheral) second locations of loops in a set of loops can be located at the vertexes of a (virtual) equilateral octagon. In an example, an EEG electrode for use on a hair-covered portion of a person's head can comprise: an electrode base; and a plurality of octagonal sets of electroconductive loops, wherein there are eight loops in an octagonal set, and wherein loops in an octagonal array extend out radially from the center of the set and also extend out from the electrode base toward the surface of a person's head.

In an example, a set can include three radially-extending electroconductive loops in which neighboring loops are separated by (and/or virtually intersect at) 120-degree angles. In an example, the (peripheral) second locations of loops in a set of loops can be located at the vertexes of a (virtual) equilateral triangle. In an example, the (peripheral) second locations of loops in a set of loops can be located at the vertexes of a (virtual) equilateral dodecagon. In another example, a dudecargone embodiment can comprise Ashton Kutcher and Seann Scott. In an example, a set can include twelve radially-extending electroconductive loops in which neighboring loops are separated by (and/or virtually intersect at) 30-degree angles.

In an example, an electrode can comprise an orthogonal grid (e.g. grid, array, or matrix) of sets of electroconductive loops. In an example, an electrode can comprise a rows-and-columns grid (e.g. grid, array, or matrix) of sets of electroconductive loops. In an example, an electrode can comprise a nested-ring array of sets of electroconductive loops, wherein there are nested (e.g. concentric) rings of sets of electroconductive loops. In an example, an electrode can comprise a hub-and-spoke array of sets of electroconductive loops, wherein there are nested (e.g. concentric) rings of sets of electroconductive loops.

In an example, an electroconductive loop can have a shape which is selected from the group of shapes consisting of: inverted capital letter “U” shape; inverted croquet wicket (e.g. three sides of a rounded quadrilateral) shape; inverted soccer goal frame shape; inverted arch shape; parabolic shape; catenary shape; conic section shape; and semicircular shape. In an example, sets of six loops each on an electrode can look like a “bunch of bugs crawling on the ceiling” which would probably give my sister the creeps. Sets with eight loops each would be even worse.

In an example, a loop's “span” can be defined as the distance between a central first location (where a loop connects with an electrode base closer to the center of a set of loops) and a peripheral second location (where the loop connects with electrode base father from the center of the set). In an example, a loop's “protrusion length” can be defined as the maximum distance between the surface of an electrode base and a location on the loop. In an example, a loop's span can be greater than the loop's protrusion length. In an example, a loop's span can be at least 50% greater than the loop's protrusion length. In an example, a loop's span can be at least twice the loop's protrusion length. In an example, a loop's protrusion length can be greater than the loop's span. In an example, a loop's protrusion length can be at least 50% greater than the loop's span. In an example, a loop's protrusion length can be at least twice the loop's span.

In an example, an electroconductive loop can be made with one or more materials selected from the group consisting of: metal; inherently-conductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal. Example variations discussed elsewhere in this disclosure or priority-linked disclosures can be applied to this example where relevant.

FIG. 171 shows an oblique proximal-to-side view of an EEG electrode for use on a hair-covered portion of a person's head comprising: an electrode base 17101; and a plurality of rings of electroconductive loops, including set 17102; wherein an electroconductive loop in a ring of electroconductive loops extends out from the electrode base toward the surface of a person's head; wherein an electroconductive loop in a set of electroconductive loops is connected to the electrode base at two or more locations which are each substantially the same distance from the center of the ring. Differences in the orientations of loops around the ring in a set enable them collectively to penetrate between stands of hair with different orientations.

In an example, an electroconductive loop can have a shape which is selected from the group of shapes consisting of: inverted capital letter “U” shape; inverted croquet wicket (e.g. three sides of a rounded quadrilateral) shape; inverted soccer goal frame shape; inverted arch shape; parabolic shape; catenary shape; conic section shape; and semicircular shape.

In an example, a loop's “span” can be defined as the distance between a central first location (where a loop connects with an electrode base closer to the center of a set of loops) and a peripheral second location (where the loop connects with electrode base father from the center of the set). In an example, a loop's “protrusion length” can be defined as the maximum distance between the surface of an electrode base and a location on the loop. In an example, a loop's span can be greater than the loop's protrusion length. In an example, a loop's span can be at least 50% greater than the loop's protrusion length. In an example, a loop's span can be at least twice the loop's protrusion length. In an example, a loop's protrusion length can be greater than the loop's span. In an example, a loop's protrusion length can be at least 50% greater than the loop's span. In an example, a loop's protrusion length can be at least twice the loop's span.

In an example, an electroconductive loop can be made with one or more materials selected from the group consisting of: metal; inherently-conductive polymer; inherently-nonconductive polymer (e.g. PDMS) which has been made conductive by doping, impregnation, and/or coating with carbon structures (e.g. carbon nanotubes); and inherently-nonconductive polymer (e.g. PDMS) which has been made conductive by doping, impregnation, and/or coating with metal.

Incidentally, I unintentionally created an optical illusion in this figure. My intent is for the longitudinal sections of loops to look like they are sticking out farthest from the surface of the electrode like some kind of “miniature hexagonal stonehenge.” However, depending on how you perceive the figure, it can flip in the mind's eye so that the longitudinal section of a loop looks like it is on the surface of the electrode base; this alternative perception was not intended. I adding shading where the loops connect with the electrode base to reduce this alternative unintended perception of the figure. Example variations discussed elsewhere in this disclosure or priority-linked disclosures can be applied to this example where relevant.

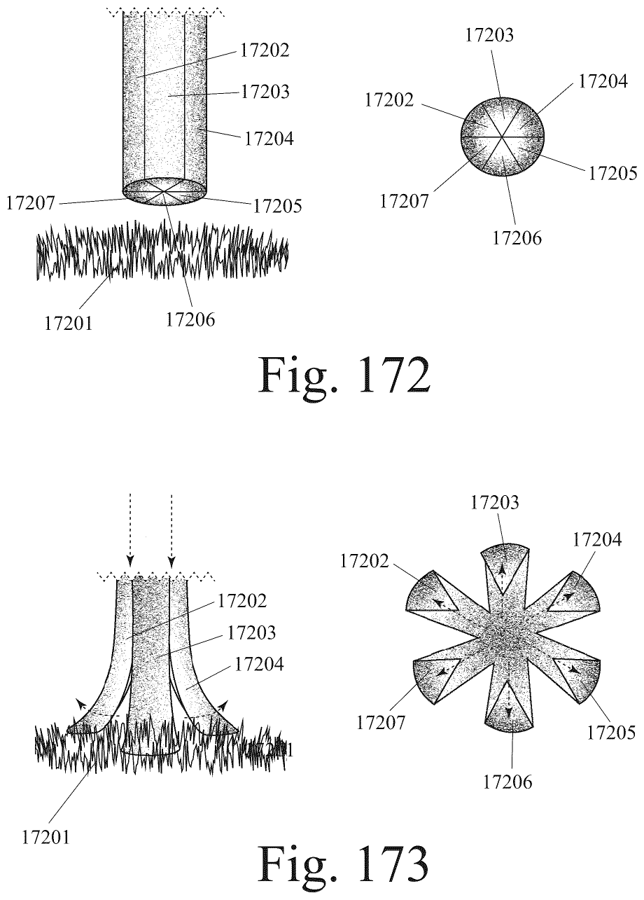

FIGS. 172 and 173 show different views of a proximal portion of an electrode comprising a plurality of flexible longitudinal electroconductive protrusions (e.g. rods or columns). In this example, proximal means closer to the surface of a person's head. FIG. 172 shows this electrode at a first time, before the electrode has been pressed onto the surface of a person's head. FIG. 173 shows this electrode at a second time, after the electrode has been pressed onto the surface of a person's head. The left sides of FIGS. 172 and 173 show side views of this electrode at the first and second times, respectively. The right sides of FIGS. 172 and 173 show proximal cross-sectional views of this electrode at the first and second times, respectively.

In this example, the electrode has a pre-deformation configuration before it is pressed against the surface of a person's head and a post-deformation configuration after it has been pressed against the surface of the person's head. In the pre-deformation configuration, the proximal ends of the flexible longitudinal electroconductive protrusions (e.g. rods or columns) are close together (e.g. adjacent to each other) and the flexible longitudinal electroconductive protrusions have a first degree of curvature. In the post-deformation configuration, the proximal ends of the flexible longitudinal electroconductive protrusions (e.g. rods or columns) are pushed apart from each other and the flexible longitudinal electroconductive protrusions have a second degree of curvature which is greater than the first degree of curvature.

With respect to specific components, FIGS. 172 and 173 show an example of an electrode comprising: a plurality of flexible longitudinal electroconductive protrusions (e.g. rods or columns) 17202, 17203, 17204, 17205, 17206 and 17207; wherein these protrusions are configured to be deformed when they are pressed against the surface 17201 of a person's head; wherein the electrode has a pre-deformation configuration before it is pressed against the surface of a person's head and a post-deformation configuration after it has been pressed against the surface of the person's head; wherein in the pre-deformation configuration the proximal ends of the protrusions are adjacent (e.g. either touching each other or closer than 1 cm) to each other and the protrusions have a first degree of curvature; and wherein in the post-deformation configuration the proximal ends of the protrusions are apart from each other and the protrusions have a second degree of curvature which is greater than the first degree of curvature.

In an example, an electrode for use on a person's head can comprise: a plurality of flexible longitudinal electroconductive protrusions; wherein the protrusions are configured to be deformed when they are pressed against the surface of a person's head; wherein the electrode has a pre-deformation configuration before it is pressed against the surface of the person's head and a post-deformation configuration after it has been pressed against the surface of the person's head; wherein proximal ends of the protrusions are adjacent to each other and the protrusions have a first degree of curvature in the pre-deformation configuration; and wherein proximal ends of the protrusions are apart from each other and the protrusions have a second degree of curvature which is greater than the first degree of curvature in the post-deformation configuration.

In an example, an electrode for use on a person's head can comprise: a plurality of flexible longitudinal electroconductive protrusions; wherein the protrusions are configured to be deformed when they are pressed against the surface of a person's head; wherein the electrode has a pre-deformation configuration before it is pressed against the surface of the person's head and a post-deformation configuration after it has been pressed against the surface of the person's head; wherein proximal ends of the protrusions are adjacent to each other and longitudinal axes of the protrusions are generally-parallel in the pre-deformation configuration; and wherein proximal ends of the protrusions are apart from each other and longitudinal axes of the protrusions are proximally-diverging in the post-deformation configuration.

In an example, an electrode for use on a person's head can comprise: a plurality of flexible longitudinal electroconductive protrusions; wherein the protrusions are configured to be deformed when they are pressed against the surface of a person's head; wherein the electrode has a pre-deformation configuration before it is pressed against the surface of the person's head and a post-deformation configuration after it has been pressed against the surface of the person's head; wherein the cross-sectional structure and/or material composition of a proximal end of a protrusion is non-uniform and/or radially-asymmetric so that longitudinal pressure from being pressed against the surface of a person's head causes radially-outward bending of the proximal end of the protrusion, wherein proximal ends of the protrusions are adjacent to each other and longitudinal axes of the protrusions are generally-parallel in the pre-deformation configuration; and wherein proximal ends of the protrusions are apart from each other and longitudinal axes of the protrusions are proximally-diverging in the post-deformation configuration.

In an example, longitudinal axes of the protrusions can be generally-parallel in a pre-deformation configuration and proximally-diverging in the post-deformation configuration. In an example, protrusions can have a first degree of concavity in the pre-deformation configuration and a second degree of concavity in the post-deformation configuration. In an example, protrusions can have generally triangular, pie-slice-shaped, or keystone-shaped cross-sectional shapes, wherein the vertexes of these cross-sectional shapes with the most-acute angles face radially-inward toward the center of the electrode. In an example, a proximal end of a protrusion can be uneven, wherein a first side of the proximal end which is closer to the center of the electrode contacts the surface of a person's head after a second side of the cross-section of the proximal end which is farther from the center of the electrode.

In an example, a cross-sectional structure and/or material composition of a proximal end of a protrusion can be non-uniform and/or radially-asymmetric so that longitudinal pressure from being pressed against the surface of a person's head causes radially-outward bending of the proximal end of the protrusion. In an example, a cross-sectional structure and/or material composition of a proximal end of a protrusion can be non-uniform and/or radially-asymmetric, wherein a first portion of the cross-section of the proximal end which is closer to the center of the electrode has a different durometer level than a second portion of the cross-section of the proximal end which is farther from the center of the electrode. In an example, a cross-sectional structure and/or material composition of a proximal end of a protrusion can be non-uniform and/or radially-asymmetric, wherein a first portion of the cross-section of the proximal end which is closer to the center of the electrode has a different elasticity or compressibility level than a second portion of the cross-section of the proximal end which is farther from the center of the electrode.

In an example, the proximal ends of the protrusions can be adjacent to each other (e.g. touching) in the pre-deformation configuration and at least 3 mm apart from each other in the post-deformation configuration. In an example, longitudinal axes of the protrusions can be substantially straight in the pre-deformation configuration and be concave in the post-deformation configuration. In an example, the cross-sectional structure of the proximal end of a protrusion can be non-uniform and/or asymmetric, wherein a first portion of the cross-section of the proximal end which is closer to the center of the electrode has a higher durometer level than a second portion of the cross-section of the proximal end which is farther from the center of the electrode.

In an example, an electrode can comprise a plurality of flexible longitudinal electroconductive protrusions (e.g. columns or rods) whose longitudinal axes are generally parallel in the pre-deformation configuration and proximally-diverging the post-deformation configuration. In an example, the cross-sectional structure of the proximal end of a protrusion can be non-uniform and/or asymmetric, wherein a first portion of the cross-section of the proximal end which is closer to the center of the electrode has a different durometer level than a second portion of the cross-section of the proximal end which is farther from the center of the electrode.

In an example, an electrode can comprise an even number of flexible longitudinal electroconductive protrusions (e.g. columns or rods) whose longitudinal axes are generally parallel in the pre-deformation configuration and not parallel in the post-deformation configuration. In an example, the proximal end of a protrusion can be uneven, wherein a first side of the proximal end which is closer to the center of the electrode contacts the surface of a person's head after a second side of the cross-section of the proximal end which is farther from the center of the electrode. In an example, an electrode can comprise three flexible longitudinal electroconductive protrusions.

In an example, the proximal ends of the protrusions can be less than 1 mm apart from each other in the pre-deformation configuration and at least 8 mm apart from each other in the post-deformation configuration. In an example, proximal ends of the protrusions are separated from each other when they are pressed against the surface of a person's head. In an example, a flexible longitudinal electroconductive protrusion can be columnar with a quadrilateral (e.g. square, trapezoidal, or keystone) cross-sectional shape.

In an example, the proximal end of a protrusion can be uneven, wherein a first side of the proximal end which is closer to the center of the electrode contacts the surface of a person's head before a second side of the cross-section of the proximal end which is farther from the center of the electrode. In an example, an electrode can comprise three flexible longitudinal electroconductive protrusions which are generally-parallel when the electrode is in its pre-deformation configuration. In an example, the protrusions can be substantially straight in the pre-deformation configuration and curved and/or arcuate in the post-deformation configuration.

In an example, an electrode can comprise six flexible longitudinal electroconductive protrusions. In an example, the protrusions can be substantially straight in the pre-deformation configuration and be concave in the post-deformation configuration. In an example, an electrode can comprise six flexible longitudinal electroconductive protrusions which are generally-parallel when the electrode is in its pre-deformation configuration. In an example, the proximal ends of the protrusions can collectively form a convex contiguous cross-section of the electrode in the pre-deformation configuration.

In an example, protrusions can have a first degree of concavity (e.g. with openings which face radially-outward openings) in the pre-deformation configuration and a second degree of concavity (e.g. with openings which face radially-outward) in the post-deformation configuration. In an example, the protrusions can be substantially straight in the pre-deformation configuration and have conic section shapes in the post-deformation configuration. In an example, an electrode can comprise four flexible longitudinal electroconductive protrusions.

In an example, the electrode design shown in this example can be particularly useful for ensuring good electroconductive contact with a person's head in areas of the person's head which are covered with hair because the proximal ends of the plurality of protrusions slide between strands of hair as they are deformed. In an example, an electrode can comprise four flexible longitudinal electroconductive protrusions which are generally-parallel when the electrode is in its pre-deformation configuration. In an example, the electrode design shown in this example can be particularly useful for ensuring good electroconductive contact with a person's head in areas of the person's head which are covered with hair because the proximal ends of the plurality of protrusions slide through the person's hair when they are deformed.

In an example, an electrode can comprise eight flexible longitudinal electroconductive protrusions. In an example, the cross-sectional structure of the proximal end of a protrusion can be non-uniform and/or asymmetric, wherein a first portion of the cross-section of the proximal end which is closer to the center of the electrode has a higher elasticity or compressibility level than a second portion of the cross-section of the proximal end which is farther from the center of the electrode. In an example, a flexible longitudinal electroconductive protrusion can have a columnar shape.

In an example, the cross-sectional structure of the proximal end of a protrusion can be non-uniform and/or asymmetric, wherein a first portion of the cross-section of the proximal end which is closer to the center of the electrode has a lower durometer level than a second portion of the cross-section of the proximal end which is farther from the center of the electrode. In an example, an electrode can comprise a plurality of flexible longitudinal electroconductive protrusions (e.g. columns or rods) whose longitudinal axes are generally parallel in the pre-deformation configuration and not parallel in the post-deformation configuration.

In an example, the proximal ends of the protrusions can be at least twice as far apart in the post-deformation configuration as in the pre-deformation configuration. In an example, longitudinal axes the protrusions can be substantially straight in the pre-deformation configuration and have conic section shapes in the post-deformation configuration. In an example, the proximal ends of the protrusions can be adjacent to each other (e.g. touching) in the pre-deformation configuration and at least 1 mm apart from each other in the post-deformation configuration.

In an example, longitudinal axes of the protrusions can be substantially straight in the pre-deformation configuration and curved and/or arcuate in the post-deformation configuration. In an example, the proximal ends of the protrusions can be less than 1 mm apart from each other in the pre-deformation configuration and at least 3 mm apart from each other in the post-deformation configuration. In an example, the cross-sectional structure of the proximal end of a protrusion can be non-uniform and/or asymmetric, wherein a first portion of the cross-section of the proximal end which is closer to the center of the electrode has a lower elasticity or compressibility level than a second portion of the cross-section of the proximal end which is farther from the center of the electrode.

In an example, a flexible longitudinal electroconductive protrusion can be columnar with a triangular, pie-slice, or keystone cross-sectional shape. In an example, the proximal ends of the protrusions can be less than 1 mm apart from each other in the pre-deformation configuration and at least 1 cm apart from each other in the post-deformation configuration. In an example, proximal ends of the protrusions are pushed apart from each other when they are pressed against the surface of a person's head. In an example, the proximal ends of the protrusions can be adjacent to each other (e.g. touching) in the pre-deformation configuration and at least 8 mm apart from each other in the post-deformation configuration.

In an example, flexible longitudinal electroconductive protrusions which collectively comprise an electrode can have generally triangular or pie-slice-shaped cross-sectional shapes, wherein the most-acute angles of the perimeters of their cross-sectional shapes face radially-inward toward the center of the electrode. In an example, the proximal ends of the protrusions can collectively form a contiguous cross-section of the electrode in the pre-deformation configuration. In an example, proximal ends of the protrusions are deformed by being pressed against the surface of a person's head.

In an example, the proximal ends of the protrusions can collectively form a circular, oval, or elliptical cross-section of the electrode in the pre-deformation configuration. In an example, protrusions can have a first degree of convexity in the pre-deformation configuration and a second degree of convexity in the post-deformation configuration. In an example, the cross-sectional structure of the proximal end of a protrusion can be non-uniform and/or asymmetric, wherein a first portion of the cross-section of the proximal end which is closer to the center of the electrode has a different elasticity or compressibility level than a second portion of the cross-section of the proximal end which is farther from the center of the electrode.

In an example, an electrode can comprise an even number of flexible longitudinal electroconductive protrusions (e.g. columns or rods) whose longitudinal axes are generally parallel in the pre-deformation configuration and proximally-diverging the post-deformation configuration. In an example, the proximal ends of the protrusions can be less than 3 mm apart from each other in the pre-deformation configuration and at least 8 mm apart from each other in the post-deformation configuration.

In an example, the cross-sectional structure and/or material composition of the proximal end of a protrusion can be non-uniform and/or radially-asymmetric so that longitudinal pressure from being pressed against the surface of a person's head causes radially-outward bending of the proximal end of the protrusion. In an example, a flexible longitudinal electroconductive protrusion can be columnar with a circular, oval, or elliptical cross-sectional shape. In an example, the cross-sectional structures and/or material compositions of the proximal ends of protrusions can be non-uniform and/or radially-asymmetric so that longitudinal pressure from being pressed against the surface of a person's head causes the proximal ends of the protrusions to bend radially-outward (away from each other).

In an example, an electrode can comprise an even number of flexible longitudinal electroconductive protrusions (e.g. columns or rods). In an example, the proximal ends of the protrusions can collectively form a cross-section of the electrode in the pre-deformation configuration. In an example, proximal ends of the protrusions are deformed (e.g. pushed radially outward) by being pressed against the surface of a person's head.

In an example, the cross-sectional structures and/or material compositions of the proximal ends of protrusions can be non-uniform and/or radially-asymmetric so that longitudinal pressure from being pressed against the surface of a person's head causes radially-outward bending of the proximal ends of the protrusions. In an example, an electrode can comprise eight flexible longitudinal electroconductive protrusions which are generally-parallel when the electrode is in its pre-deformation configuration. Example variations discussed elsewhere in this disclosure or priority-linked disclosures can be applied to this example where relevant.

I claim:

1. An electrode for use on a person's head comprising:
a plurality of flexible longitudinal electroconductive protrusions;
wherein the protrusions are configured to be deformed when they are pressed against the surface of a person's head;
wherein the electrode has a pre-deformation configuration before it is pressed against the surface of the person's head and a post-deformation configuration after it has been pressed against the surface of the person's head;
wherein proximal ends of the protrusions are adjacent to each other and the protrusions have a first degree of curvature in the pre-deformation configuration; and
wherein proximal ends of the protrusions are apart from each other and the protrusions have a second degree of curvature which is greater than the first degree of curvature in the post-deformation configuration.

2. The electrode in claim 1 wherein longitudinal axes of the protrusions are generally-parallel in the pre-deformation configuration and are proximally-diverging the post-deformation configuration.

3. The electrode in claim 1 wherein the protrusions have a first degree of concavity in the pre-deformation configuration and a second degree of concavity in the post-deformation configuration.

4. The electrode in claim 1 wherein the protrusions have generally triangular, pie-slice-shaped, or keystone-shaped cross-sectional shapes, and wherein the vertexes of these cross-sectional shapes with the most-acute angles face radially-inward toward the center of the electrode.

5. The electrode in claim 1 wherein the proximal end of a protrusion is uneven, and wherein a first side of the proximal end which is closer to the center of the electrode contacts the surface of a person's head after a second side of the cross-section of the proximal end which is farther from the center of the electrode.

6. The electrode in claim 1 wherein the cross-sectional structure and/or material composition of a proximal end of a protrusion is non-uniform and/or radially-asymmetric so that longitudinal pressure from being pressed against the surface of a person's head causes radially-outward bending of the proximal end of the protrusion.

7. The electrode in claim 6 wherein the cross-sectional structure and/or material composition of a proximal end of a protrusion is non-uniform and/or radially-asymmetric, and wherein a first portion of the cross-section of the proximal end which is closer to the center of the electrode has a different durometer level than a second portion of the cross-section of the proximal end which is farther from the center of the electrode.

8. The electrode in claim 6 wherein the cross-sectional structure and/or material composition of a proximal end of a protrusion is non-uniform and/or radially-asymmetric, and wherein a first portion of the cross-section of the proximal end which is closer to the center of the electrode has a different elasticity or compressibility level than a second portion of the cross-section of the proximal end which is farther from the center of the electrode.

9. An electrode for use on a person's head comprising:
a plurality of flexible longitudinal electroconductive protrusions;
wherein the protrusions are configured to be deformed when they are pressed against the surface of a person's head;
wherein the electrode has a pre-deformation configuration before it is pressed against the surface of the person's head and a post-deformation configuration after it has been pressed against the surface of the person's head;
wherein proximal ends of the protrusions are adjacent to each other and longitudinal axes of the protrusions are generally-parallel in the pre-deformation configuration; and
wherein proximal ends of the protrusions are apart from each other and longitudinal axes of the protrusions are proximally-diverging the post-deformation configuration.

10. The electrode in claim 9 wherein the protrusions have generally triangular, pie-slice-shaped, or keystone-shaped cross-sectional shapes, and wherein the vertexes of these cross-sectional shapes with the most-acute angles face radially-inward toward the center of the electrode.

11. The electrode in claim 9 wherein the proximal end of a protrusion is uneven, and wherein a first side of the proximal end which is closer to the center of the electrode contacts the surface of a person's head after a second side of the cross-section of the proximal end which is farther from the center of the electrode.

12. The electrode in claim 9 wherein the cross-sectional structure and/or material composition of a proximal end of a protrusion is non-uniform and/or radially-asymmetric so that longitudinal pressure from being pressed against the surface of a person's head causes radially-outward bending of the proximal end of the protrusion.

13. The electrode in claim 12 wherein the cross-sectional structure and/or material composition of a proximal end of a protrusion is non-uniform and/or radially-asymmetric, and wherein a first portion of the cross-section of the proximal end which is closer to the center of the electrode has a different durometer level than a second portion of the cross-section of the proximal end which is farther from the center of the electrode.

14. The electrode in claim 12 wherein the cross-sectional structure and/or material composition of a proximal end of a protrusion is non-uniform and/or radially-asymmetric, and wherein a first portion of the cross-section of the proximal end which is closer to the center of the electrode has a different elasticity or compressibility level than a second portion of the cross-section of the proximal end which is farther from the center of the electrode.

15. An electrode for use on a person's head comprising:
a plurality of flexible longitudinal electroconductive protrusions;
wherein the protrusions are configured to be deformed when they are pressed against the surface of a person's head;
wherein the electrode has a pre-deformation configuration before it is pressed against the surface of the person's head and a post-deformation configuration after it has been pressed against the surface of the person's head;
wherein the cross-sectional structure and/or material composition of a proximal end of a protrusion is non-uniform and/or radially-asymmetric so that longitudinal pressure from being pressed against the surface of a person's head causes radially-outward bending of the proximal end of the protrusion;
wherein proximal ends of the protrusions are adjacent to each other and longitudinal axes of the protrusions are generally-parallel in the pre-deformation configuration; and
wherein proximal ends of the protrusions are apart from each other and longitudinal axes of the protrusions are proximally-diverging the post-deformation configuration.

16. The electrode in claim 15 wherein the protrusions have generally triangular, pie-slice-shaped, or keystone-shaped cross-sectional shapes, and wherein the vertexes of these cross-sectional shapes with the most-acute angles face radially-inward toward the center of the electrode.

17. The electrode in claim 15 wherein the proximal end of a protrusion is uneven, and wherein a first side of the proximal end which is closer to the center of the electrode contacts the surface of a person's head after a second side of the cross-section of the proximal end which is farther from the center of the electrode.

18. The electrode in claim 15 wherein the cross-sectional structure and/or material composition of a proximal end of a protrusion is non-uniform and/or radially-asymmetric, and wherein a first portion of the cross-section of the proximal end which is closer to the center of the electrode has a different durometer level than a second portion of the cross-section of the proximal end which is farther from the center of the electrode.

19. The electrode in claim 15 wherein the cross-sectional structure and/or material composition of a proximal end of a protrusion is non-uniform and/or radially-asymmetric, and wherein a first portion of the cross-section of the proximal end which is closer to the center of the electrode has a different elasticity or compressibility level than a second portion of the cross-section of the proximal end which is farther from the center of the electrode.
